SEQUENCE LISTING (22 sequences)

SEQ ID NO: 1 - DNA, 20536 bp, synthetic construct
  misc_feature 1..20536: note = Shewanella sp.
  source 1..20536: mol_type = other DNA
aatgtttaaccgaagatgaataagacaagttaaccataatcggcaagtaaggcttctatgttgctcagttgcgacgttagcattatggaaatctcggtgttaccagcatgccctaagctcacggcaaaacggtactcctcggtcaaacgcccaaaataggtttgccaatacaatgaacttgcatcaatggtttgcagcgggatattaggatagacactcagcaaaggcgccacaccatagaacggcatcaacgctgcatcttggcatctaagctcaaccgatccggccaccaattgctctaatggctttagatcaaagccgaaggatttaagcgactgcgctaaacctaggccagtcgccttaatataggattcctttaacgcccataaatcgaaaaagcgctgccgctgactcgcttcatcgacaagggtcaacaaagccgcagtttcttgggcggaaaaataatgatttaggattggatagatatccgtcttcggccgcgatctttcaatatcgaccccaaataaaccagactcaacaccatgaaactgagtcacaccaatcaataaccaatcgccgctatggcttaggttaaactcaagccccgtttgctgttgcaattttgccgttaaactcggcttacccttagcgccatattcaaaacaccactcgtgtggcgataaatccgcatagcgagataacacggcacgcaacgcagccctgacctgtaatcccttcatttgcgccttgggatcgcgataacgcgcgaccttagcgagttcatcatcacttaaccaagactgagcgagtgatgtttgcggcaatgaaatatcactcagtggaataaaaaatagatccacattcacgccgtttttgctagatgttctggagtcggtcatttgagggccttgcaggacaaagccttgcggctgatggttatcattgtctggctttttggagagcacttagcaacattcttatctcggtattgtctaggttcatcgttatctgagttcattatgcatcttaaatagtttcaactaagccctatttcaattcttagcagcactctaattagagcaattcttagaacgactctgaacgccatcctaagcgcaaatttttgctgcaattgcgccaaggttttgcgcccaaattaacaggtttagccgcacagtctgattccttgcagaggaggcttaagccaatattgggctattttaccccgcgaactcaaaaaagcggtctttatcgcccctaaaaatttaattcttccactatcaaagtattaaaaagcccaagcggtcagcgaaaacccaacataacgcacagaaaataccattactcaacgaaagccaataaattccagtcccagatgattgtatcgacccgaaacctcaggtaatctgcatccccattaagtgacacagatgataagcacaggttttacgatgagtgtcactatgttcttcaagagtgccacagctgactcagccaaaatggcttctcagtcccaatagattatgtcggatgataattattattaatggcagctcgcgactctcaatctaaagcgccgtcttacgcccccaagagcacgactcaagcggcgaaaaaagacagcgcacctcggcatcgcaacgccaccaccacgcctgaaatgcgactctttatccagcaatcggatctgagcgtcagccagctggcaaaaattttaaatatcaccgaggcaacggtgcgtaaatggcggaagcgtgagtccattagtaacagttcgaacacgccccatcacctcaataccacgctcaccccgatggaagaatatgtggtggtgggattacgctatcaactcaaactcaccttagatagattacttcaggttacccaaacctttattaatcccaatgtgtcgcgctccggccttgcccgttgcttaaagcgctacggcatatcgcggctcgatgaatttgaagcaccgcaagtccccgaacgctattttaatcaattgcctgtgacgcaaggcagcgatatccaaacctatacggtcaatccagaaaccttggccaaagccctcgcattgccgagtaccgacggcaatactgtggtgcaagtagtgtcactcaccataccgccgcagctcactgagcaagcgccaagctcagtactattaggcgtagacacagcaagtgattggatttacctcgatatttatcaagacagcaatacccaagcgaccaatagatatatcgcctatgtcctcagacatgggccgtttcatttacgaaagttgctcgttcgcaactatcacaccttcttagcccgttttcccggtgcccatggcacgccaaagacaagcgcggctggatcccaaaacaaggtcaccgtatccaggtcgacccgtggagactctttatgagccataccccttctgtacctaattctgcgactgagtcaaaaaaagataaacgactcaacaaacgtttaaaagatatgcccgttgccatcgtcggcatggccagcatcttcgctaactcgcgttatttaaataaattttgggatttgatcagcgaaaaaatcgatgccattactgacatcccagacacccattggcgcgccgaagattactacgatgccgacaaaagcaaagccgataaaagctactgtaaacgcggtggttttttaccagaagtcgacttcaatcctatggaattcggtctgccgcccaatattcttgagctgacagatacctcgcaactgctgtcgctgattgtcgccaaagaagtgcttgccgacgccaatctgcccaccgattatgatcgcgatcgtattggcatcacgctaggcgtgggcggtggtcaaaaaatcagtcagagcttgaactcgcgcctgcaatatccagtgcttaaaaaagtattcaaaagcagcggcctgagcgatgaagacagcgaaatgctgatcaaaaagttccaagaccaatatatccactgggaagaaaactctttcccagggtccttaggcaacgtgattgcaggccgtatcgccaaccggtttgattttggcggcatgaactgcgtagtcgatgcagcctgtgcgggctcgcttgccgctatgcgtatggcgttgactgaactgaccgaaggtcgcagcgacatgatgttaaccggcggtgtctgtaccgacaactcgccgtccatgtacatgagcttctcaaaaacgcctgccttcaccaccaatgaaaccattcaaccctttgatatcgattcaaaaggcatgatgatcggcgaaggtatcggcatggtcgcgcttaaacgccttgaagatgccgagcgcgatggcgaccgaatttatgccgtcatcaaaggtgttggtgcctcatcggacggtaagtttaagagtatttatgcgccgcgccctgagggccaagctaaagctctcgaacgcgcttacgacgatgcaggttttgccccgcacagcattggcttagttgaagcccatggcacgggtactgccgcaggtgatgtggctgaatttaatggcttaaaatcagtatttgcccaaggtaacgacacaaatcaacatatcgcgttaggttcagtgaaatcccaagtgggccacactaaatccacagcaggcactgctggggtgatcaaagccgcgctagcgctgcaccacaaggtattgcctgcgaccattaacgttagcaagcctaatccaaaactgaatatcgaaagctcaccattctatttaaataccgaaactcgcccatggttccagcgcgccgatgcaacgccgcgccgtgcaggtgtaagctcctttggttttggcggcacgaacttccatctcgtattagaagaatacaaacctgagcacagccgcgatgagcaatatcgtcagcgcagtgtgccgcagacactattgtttgccgcagccaataaagccgcgctgcttagcgagttaaaagctgcgctgagccaaagcgtgaacacaagcgcaaacgcgaataagagcagcgctgcgagcctgaacgctatcgctcagcaatatccgctgcgcccgctggcagacacagatgcccgtttaggttttgtggctaaggatatcgctcagctgcaagctcagttaaatcaagccatctctcatctagaaagcagtgcgcatttagaaagcgggcacctagaaaccaatgcaattgaagtgtggcaattaccttccggtatcagctaccgctctcatgccttagtggccaagaatgactccaataaagtggccgccctgtttgccggtcaaggttcacagtacctgaacatgggccgcgaacttgcctgccatttcccggaaatgcgccaacaagtgacggccagcgataaagtatttgctcaccacggtcaaacaccgttatcgaacatcctctaccctattccagcattcgatgccgatgcggtgaaagcccaagaagcggcgctgaccaataccctgttcgctcaaagcgccataggcgcggtttcaatggcgcaatattcgctattaacgcaagcgggttttgcacccgatatggtggcaggtcacagctttggtgaactgtcagccctttgcgcggcaggcgtgatttccaatgaagattacgtcgaactggccttcgcccgtgggcacgccatggcgcaagtgccgagcgataccgcgagtaaagccgcagacactggaactcaaactgacttagggacaatgttcgccatcattcttaaacaaaagaatgatatcgatgcgatcaatagctgtttagcccagtttgatgacgttaaaattgccaactacaacgcgccgacgcaattagtcatcgcaggtggcacagagcaaactcagctggccgcgaaagccattagtgaactgggctttaaagcgattgccctgcctgtctcaggcgcgttccataccccattggttggacacgcacaaaagccatttgccaaagccattgataaagctaaattcagcgcgccttcagtcgcactttatgccaacggtacgggtcaattgcacccaagcgatggcaaagccattaaagcagaattcaaacaacacatgctgcaatcggttcgctttagcgaacaactgcaggccatgtatgacgctggcgcccgtgtgtttgttgagtttggccctaagaacatactgcaaaagttagttgaaaataccttaggtgaacacttaaatgagctttgccttgtcagcatgaacccgaatcctaagggcgacagtgacagtcaattacgcttagccgcagtgcaattagccgtagccggcgtggcgttaactgaggttgacccctatcaagccgtcacgtcacaagaaattgccgaacgtgaagcgccatcagcgatgaacatcaaactcactgccactaaccatattagtgctgcaaccaaagctaagatggctaaatccctcgccacaggcagcgtgactagccaagtgcaatatgtggatcgcatcgttgaaacggttgtcgaaagagaagtcgaaaaaatcgttgagaaagaagtcatcgtcgaaaaagttgttgaaaaaataatagaagtggaagcgaatcaagtggcaactgttgaaatgaaacaaagatccccaagcgtgacgcaaggcttaagcaatcagcagcaagcgacggcgcagttaaacccaagcacacctaacgtcagcagcgatgcattaacggcatttttcagcgcacaatctcaagcggcgcagttgcatcaacagtttttagctatcccgcagcaatacggcgataccttcacgacactgatgactgaacaagccaagatggcaagccttggcattgcaatcccagaaagcctgcaacgctcgatggaaatgttccaccagcatcaagcgcaaaccctgcaaagccacgccgaatttatgcaactgcaaagcagtagcagccaagcggccttggccatgttaaacaatgcaccgattaactttactccagcagttgcaagtcagccacgagcaactgcgccggctccagcaccttttgctgcatcgacagttgcgcacaatgccgcaccagtagcggctcaggctgttgcgacgagaccagcaattaacacacctgttgcacctgttgtgcaaaccgcgcctgttgctcatgcgcctgctgcgacagtacaagttgcgccagcaggtcctgcattagtcatgcctgctgtagtgatgccagaagtgacacctgtggcaccagcaacgagcggtttaagcactgcgttagtacaacaaaccatgatggctgtggtggccgacaagacaggctaccccactgaaatgctggaacttggcatggatatggaagccgatctaggtatcgattcgattaagcgcgttgaaattttaggcacagtacaagacgaactgccgggcttacctgaactcagcccagaagatctggccgagtgccgtaccttaggtgaaatcgttgactacatgaacagcaaactgcctgcggctggccaagttgtagtttctacaacagcgccaacagctcctgccgccaacggtttaagcgctgcgttagtacaacaaaccatgatggctgtggtggctgacaagacaggctatccgactgaaatgctggagctcagcatggatatggaagccgatctaggtatcgattccattaagcgcgttgaaattttaggtacagtacaagacgaactgccgggtttaccagagctcagcccagaagatttggctgagtgccgtaccttaggtgaaatcgttgactacatgaacagcaaactgccagcggctggccaagttgtagtttctacaacagcgccaacagctcctaccgctaatggtttaagcgctgcacttgttcaacaaaccatgatggctgtggttgccgacaagacaggctaccccactgaaatgctggagctcagcatggatatggaagccgatctcggtatcgattcaattaagcgcgttgaaatcttaggtacagtgcaagacgagctgccgggtttacctgagctcagcccagaagatttggccgagtgtcgcaccttgggtgaaatcgttaactacatgaacagcaaactgccagctgtaggatcagttatcgtagcaactcatgtgacaactgccgcgcctgccgccagcggtttaagcgctgcgttagtgcaacaaactatgatggcagtggtggccgacaagacaggctaccccactgaaatgctggaactcagcatggatatggaagccgatctaggtatcgattctatcaagcgcgttgaaatcttaggcacagtgcaagatgagctaccgggcttacctgagctgaatcctgaagatctggccgagtgtcgcaccttaggcgaaatcgttgactacatgaacagcaaattgccagtagttagcgcggtagcagttgcaccagcggcaaacgttgaagcaacgccagcacaagttccagcgaataacggtttaagcgctgaaaaagtccaacaaaccatgatgtcagtggtggccgacaagacaggctaccccactgaaatgctagaactgagcatggatatggaagccgatttaggcatagattccatcaagcgcgttgaaatcttaggcacagtacaagacgaactaccgggtttgcctgaactcaatcctgaagatctggccgagtgtcgcaccttaggtgaaatcgttagctatatgcaaagcaagctaggccaaaacgcggtaagccagaatgccgcgccagttagcgcagtcgttcagcctgaacctactatcgatttaccgccacacagtgaggtagtgctaaaaaagttgccagcggcggctgagctaacgcaactatcgccacagcaatcgtcaaagcaatcagcgcaaaaagctctgccacgcgtttttgctaaagatgcctgcattatcattagcgatgatggtcacaatgctggcgtacttgccgagaaattgcatgctcaaggtctaacggttgcggttgtgcgctcgccagaaagccttgtagccagtgcatcaccgctcaatagccacattgccagcttcacattggcggcgattgacgatgccagcatcagcgcagtgattaatgagatcaaaagcttaggtacagttgcaggctttattcatctgcaaccacaacataaaacctcagcagatactaaaggtttagtgttgtccagtgccgccaaagcgtcggtcgagcaagcattcttgttcgccaagcacttacaaccgtttttaacaacagcagcgaccgccaatactggtagtagttttatcagcgtcagtcgtatcgacggtggttttggttatcttaaccacagccaaattgcccgcagcgagttaaaccaagcagcattggcaggtctaacgaaaaccttgagtcacgagtggccaagcgtgcattgccgcgccttagatatcgcgccagcacttgatgccaagcaactggccaatgcagtgatcgctgagctctttgcaaccgataacgtattggaagtcggtgttaaccaaggcgcagcccaagcacttgagcgtgtaaccttagttgctggcaaggctgatacccgccacagcgcggcaaacttgacgagtgcagataaaatactggtgaccggcggcgcaaaaggcgtgacctttgaatgtgccttgagtctggcaaaacgcagcaaggcgcactttatccttgctggcagaagcagccaacaagccattcccgcttgggcacagggtaagaataacagtgaacttaaagccgcagctattgcccacattcaaagcttgggcgagaaacccacgccaaaacaagtggatgccttagtatggccagttcaaagcagcctagaaattgcagccgcactgcaagcctttaccgctgtgggcgcgagcgccgagtacttaagtctcgatgttaacaatcccgatgcgatcgccagcacgatagtgccgatcaccgagttgtcgcctattactggcatcatccatggtgcgggcgtactcgccgataaacacatccaagacaaaaccttagatgagttagcacgggtgtatggcactaaggtcacaggtataagcaatctacttgctgcactggatttagataaattaaaactgattgccctattctcttcggcggcgggtttttacggcaatactggccaaagcgattacgctatgtccaacgacatactcaacaaagccgcactgcaactggcgcagcaactgccaaatgccaaagtgatgagcttcgattggggtccgtgggacggcggcatggtgaatcctgcgctgaaaaagatgtttatcgatcgcggtgtttatgtgatcccgctaaaggcaggcgctgagttatttgcgagccaattactgagcaacacaggcgcacaattgctggtcggtactgatatgcaaggcacagttccaagtgacgagcaatcaactcaaggtagcaatctaaaaaagcctgaagcggatctgaccactgacgcgtcggatccgcatgctttgcctaaagctgtaaaactaaagcgcacactggaccctaaggcaatgatctttattgaagatcactgcattaatggtaatcccgtattaccgactgtgtgcgcgattcagtggatgcgtgaagccgcctttgatgtgctcaagcagcctgtcaaagtgcaaagctacaagctgttgaagggcattattttcgacactgccgtgttacaaaatggagtgttagaaagtgcagcccccatcacgctggaacttgaacttgcaccgattgcgttaacggataaagccgcgaaagatactgacgagtgcttgagtgaacaattcagtgccttaatcagctttgaaggtcgtccgcaatatcaagccatcttagtgattgatgacgcggcgagtgattacttagccactaacagcaaagcaactgcctttgacgcgcatagcttggcagggctttctgccatcacaaccgcgagcagcctttacagcgacggcacgcttttccacggcccaagactgcaaggtattgagtctgtggtgaagtttgacgatgcgagcttagtcgccaaggtaagccttccccatgttgcgttagcggactgcgggagttttgtgcccaatctggcccctaaaggctcacaagcctttgcggaagatttgttgctgcaggcgatgctggtatgggcgagactcaaatatggcgcggcgagtttgccatcaagcattggtgagtttatttcccatgcgccgtttgcctttggcgataaagggtatctagtgctcgaggtggtaaaacactctggccgtgcccttgaagccaatattgcactgtatcatcaagatggccgcttaagctgcgagatgaacaacgccaaagtcaccatcagcaaaaaccttaacggtgcgtttttagcaaataaagttgccaataaagttgttaataaaagcattgaaagcgtggaggcaaaagtcgagtgaacttagccccccctttagcaacgacccataacggccaagctgaaacgtcgatagctgacaaaagcgctaagccactgcgtattgcagtgttgcttggcgatgccgtcaaccttgactctcactcagcccaagtattagggacgtttactgaacgtgaaagagttcagatttgcgctgccgatgccaaccaatcgacagctcatgaaccgtcagtgcatgagcaaaagtcgctcactgcgttgttaggccaagcaacaacagccattgagcaaggcaagctcgttgaacttaaattcaatgatggcaatctgccccaggctttgactcagtccctgtatttgcttgatggtttacgcgccgccaaactgcgtcttcacgcccacgcgtttattgctggctttgccgctggtaatgaaactactggtgttgtaaatgcaggtgttgctaatgcagcggcggttgctaatgctgcattagcggcggcaaagcgtaatccagctcaaaccgttcaacatcagactgtcgccaacactctcaacgaggcctttgttgcgctgcgccaaggcgtaaccgccctcgctgcgcgaacacaagcgccgcttaaaggcactactgatataaaacagacaaacgacaccaatcatcaaactggctattggtttagcgaccaacatcaagcgcgggttttatgtctaaatcttgtagcaaagaatctagcggcaacaagatcaaacccagcagataccttgctggattcaggccaagatatttgccaaagcctagtactgacccaaggcacacaagttatcgcgcccaaagctcttgttgatgaaaaccggctgtttgtgccgataagtgctgacaatattgattcactcatcgcgcaagtattgcaactagttaacactctggaaaatggcgtaggctttcacgcagactttcgcgctgagcaactcaaaccttggtttgaacgctatgatgccaacgcacctctggcattagtcttgatggcggcatcgattgatgatctcaaacttgaagctaaggccatgcttacggcgcttgaaaatgatgcggtgcgtcatcacggtcaacactttaagacgccagcgggtagctgctttaccgctaagccgctaggggatgcggggctgacctttgtatatcccggtgttggcacagtttatgccaatatgttcaataacttgcatgagtatttccccgcgctatatcaccagctagaacgtgaaggcgacttaagcgccatgctgcaatcaccgcagatttatgcggcagatgtaaaaaccgcagcgggcatgagcttaagtcagcaagcgattagtggcgtgggcgccagctacctattcactaaactgttgacccaagtctttaatattaagcctaaaatggcgctcggttactctatgggcgaagcggccatgtgggccagccttgatgtgtggcaaacgccccatgctatgatcaaggccaccgaaaatagcgatattttcaaccatgccatttctggcgagctaaccgcagtacgccgagcatggcagcttaaggataatgaatccatagtgtggaacagctttgtggtgcgcgccgatagcaacgaaatcaaagcattactgccagagtttcctcgcgcttatttagccatcattcaaggtgatacttgcgttattgccggctgcgaaacaagctgtaaagccctacttgcaaagttaggtaagcgtgggattgccgccaatcgcgtcacggcaatgcataccgcgccagccatgttagtccatgggcaagtacaagatttctatactcaagcagtgaaacctgaggcactgaaacctaaggcgctggaagcggcagcacatggctcgtctattcgctttatcagtgctgcgcaaactgcgccagtcaccgtggatagccacagcattggccgcgcgattgccgataccttttgttcgccactcgactttagtgcgctcattcaaaatgccactgagcaaggtgcaagactgtttgtcgaagtgggcgccgataggcaaaccagcacacttatcgataaaatcagccatgcccatgcaggccaaagcacggtgcacgaggcaacagtcgccattgcttgcaatgccaagggtgccgatgcgattaccagcttactcaagtgcttagctcagctgataagccacagagtgccgctgtcgctcgtgccgctcattaagccattaagtactaacgcagcccctttatcatcagcagtatcaccacaaggagaaccccagtgagttctcagcattcccctactattgataaaactaccgttcctatgattttgtcaaaaagcgcgtcaaaaattgcgattgtcggtcttgcgactcagtatcctgacgccgataatccccaaaccttctggcaaaatctgttggataaaaaagactctcgcacccaaatcagtcgcgaaaagctcaatgccaatcccgccgattaccaaggtgtgcagggtcagtctgaccgtttttactgtgataagggcggctacatccagcactttgagtttgatgccaagggctatcaactgcctgagtctgcctttgacggtttagatgaaagctttttatgggcactcgattgcagtcgtaaagcccttcaagacgcagggattgcaccaagcgatgccgtactggcgcgcacaggtatcgtaatgggaaccttgtcgttccccacggcccgctccaacgaattatttttgcctctgtatcatcaaaccgttgaaaaggcgctacaaaacaaacttaaccaaagcgcatttcagttggctgattttggcaactcagttaacgctctcaatgccaataaccaagcattaaacgtcgccaatggcgcggttgcccacacagcctcaaagctggtcagcgatgcgctcggtttgggtggcactcagttgagtctggacgccgcctgcgccagctcggtttatgcactcaaactcgcctgcgattacctaacaactggcaaggccgatatgatgcttgcgggcgcagtatcgggcgccgatcccttctttatcaacatgggattctcgattttccacgcctacccagatcacggtatttcggcgccctttgatagcaacagcaagggcttattcgccggagaaggcgctggggttttagtcttaaaacgcttagaagatgccgagcgcgatggcgataacatctacgccgtcgtcagcggcattggtttgtcgaacgacggtaaaggccagtttgtattaagccccaacagcaagggccaagtgcaagctttcgagcgcgcctatagcgcggcaaacactcagcctgccaatatcgaagtgattgaatgccacgccaccggcacgccgctcggtgataaggtcgaactcgcctcgatggagcgcttcttcgaggacaaactcgcgggctctgcggtgccgctgatcggttcggcaaaatccaacttaggccatttactcacagccgcgggcatgccggggatcatgaagatgatttttgccatgcgctcgggtcgactgccgccaagtattaacttatcagcgccgatatcctcgcctaagggcttatttagcgctaaaaatctgccaacagaattgcatgcttggccagataaagccggcaatgaccgccgccacgcgggtgtttccgtgttcggttttggcggctgtaacgcgcatttattgctggaatcctatgttgccaatacaaacaagaagaatgaacaagccgcagccgcagtaagttaccagcacacgccattaaatatcattggcttagcatcgcactttgggcctttaacctccattaatgcactggataacacgattcatgctcggcaacatgcctttatcccgctgcccgctaaacgctggaaaggcttagataaacaccctgagatcttagctaactttggtttgcctgctgtaccgcaaggcgcgtatatcgaccagttcgattttgacttcctgcgctttaaagtgccgcccaatgaggatgatcgcctgatctcccagcaactgctgctgatcaaagtggccgatgaagcgatccgtgatgccaacttaaaacccggtggcaaggttgctgtattagtagcgatggaaaccgaactggaattacaccagttccgtggccgcgtaaatctgcacactcaactggcagacagtcttaaaaaacaagggataacactcacacaagccgagtatctcgcccttgaaaagatcgccatggacagtgtgctcgatgccgccaaactgaaccaatacaccagttttattggcaacatcatggcgtcacgtatcgcatccctttgggactttaacggccctgcctttaccatttcggcggcggagcaatcggtcgcccgctgtatcgatgtggccgaaaatctgttgtcccaagaatccttagatgccgtagtgatcgccgcagtcgatttaagtggcagtttagagcaagtgatccttaaaaacgccgtatcgccagtggcatttactgccactgacacaggctggaaagtcggtgaaggtgctggcgcgttagtgctcacaacagcgcaatcaagtgcagcccagccaagtaatgccgctgacaatgctaatgccaatagcagctatggcgacagttatggtcacatcagcggccaagtatttggcgcgatttgtgacatacaaggtaatagcaacactgcgcgtatttgcgatgacttgctaacccaagccaaggtgaatagcagccagattaacttgattgaaaccagtattgcggttgagcaacttgccgattcagagctggtactcaatacgctgctgcagagtgtgaaccagcgcagccaagccgctgataccctaggccacaactttgccgctgcgggtatggcgagtattttgagcgcgctgttgcagcttaaaaatcaagtgcaacttaacaagcattcttctcaagctcagcagcacgcgctcgttgccacgtttagccaaggtaaatgctcgcagttactgctcagccaaagtgccacgcaagcatacaatttgcagcaaaggcttgaacaagatttaacgctttctgagcaaaaacacttaataaaacaagtaacgctcggtggccgcgatatctatcagcatatcctagataccccgctcgcaggccttgatgctatacaacaaaaagcccaagccatgacagcactgccagcgcgcagccaacgcaaacatttggcccaaatagcgagcaaagacacgaatagctttgcaacaagcagcccaaccacggctcaacaaaaagagacattaagcagcatgccaattaatgccctaagcaccaataaaaacgccactcaagcagagctaaaagacgcggcatttttaagcaatcagcaactcgcccgtgaagcccatttagcctttttacaaagccgtgctgaaggcttgaaactagccgatgccttgatgaaagcccagcttgccagcgagttagccatcaatggccaagctgcgccagtacaacagcaagccattgttcaggcatctgcgacagcggttcttccacatccagtgcattatcccaatcatgcaaaagtgcctgagtacacgccgccaacaccgataagcaagccttgcatttgggattatgcggatttagtcgagtacgccgaaggtgatattgctaaagtatttggcccagattacgccattatcgacagctattcgcgtcgtgtacgcctgccaacgactgactatttgctggtatctcgggtaacgaaattaaacgcccagatgaaccaatatcagccttgcactatgaccacggaatacgacattcctgtggacgcgccgtatttggtcgatggccaaatcccttgggccgtggcggtcgaatcgggtcaatgcgacttgatgctgatcagctacttaggtatcgattttgaaaataaaggcgagcgcgtgtatcgcctgctcgattgcaccctgaccttccttggcgacttaccgcgcggtggcgacaccctgcgctacgacatttccatcaatcactttgcccgcaatggcgataccttgttgttcttcttctcctacgaatgtttcgtgggcgacaagctgatcctgaaaatggatggcggctgtgccggcttcttcaccgataaagaactggccgacggcaaaggcgttattcacaccgaagctgaaatcaaagcgcgcaacctcgccttgaataatccgaataaacagcgctttaatccgctgcttgattgcgcgcaaaaccaatttgattacagtcaaatccataaactgctcggcgccgatatcggtggctgttttggcggtgcacacgcggcgcatcaagcccaatatggtttgcagccctctttatgttttgcatctgaaaaattcctgatgattgaacaggtcagcaatcttgaggtgcatggcggcgcgtggggcttaggttcagttcaaggccataagcagctcgaagccgatcattggtatttcccatgtcatttcaagggcgaccaagtgatggcgggctcgttaatggccgaaggctgtggtcaattactgcaattctttatgctgcacattggaatgcacctcggtgttaaagatggtcgtttccaaccgctcgaaaatgcgtcacaaaaagtgcgctgtcgcggtcaagtcttgccacaatcaggcctgctcacctatcgcatggaaatcaccgaaatcggtatgagcccgcgcccgtatgctaaggcgaatatcgatattttgctcaatggcaaagtggtcgtggatttccaaaaccttggagtgatgatcaaagaagaagccgaatgtactcgctaccttgcgaataatgatgccagtaaagctgacaatgctgctaaaaatgctgcttcggctgcgacgctggtttcaacagcacccgtattgtcagccgcgccgctgatggcgcagctgcccgatttaactgcgccaaccaacaaaggcgtagtgccgcttaagcatgtgtctgcgccgattgcgccagcggattcaaagtatgctaaccgcgtgcccgataccctgccgttcacgccgtaccacatgttcgaatttgccactggcgacatcgaaaactgcttcggccccgatttcagtatctatcgcggcctaattccaccgcgcacgccttgcggtgatttgcagctcacgactcgcgtcattgcgattgacggcaagcgcggcgaactgaaaaagccctcttcgtgtatcgctgaatacgaagtgcccgcaaacgcttggtattatgacaaaaacagccatcacgccgtgatgccctattcagtgctaatggaaatatcactgcagccaaacggctttatctcaggctatatgggcactaccttgggcttccccggccaagagctgtttttccgcaacttagacggtagcggtaagctgctgcgccatgtggatttacgcggcaaaaccatagtgaacgactcacgtctgttatcgactgtgattgccggcagcaacatcattcagaatttcagcttcgagttaagctgcgatggcgagcctttctaccaaggtaaagcggtatttggttacttcaagggcgatgcgctgaaaaaccaacttggcatagacaacggcaagatcactcagccttggcatgtgcaaaatggcatagtagccgatagccaaatcaatctgttagataaacaacatcgcagctttaatgcaccaaaaggtcaaccacattaccgtttagcgggcggtcaacttaactttatcgacaaggctgaaatcgtgaaagccggcggtaaggcgggccttggctatttatacgccgagcgcacgattgacccgagtgattggttcttccaattccatttccatcaagatcctgtgatgccaggctcattaggggttgaagccattatcgagctgatgcaaacctatgcgattaaccaagaccttggtgcgggttttaagagtcctaaatttgggcagattttatcggatatcaaatggaaatatcgcggtcaaattaacccattaaacaagcagatgtcgctagatgtacacattaccagcgtgacagacgacaatggcaaacgcatcattatgggcgatgccaacttgagtaaagatggcctgcggatttatgaagttaaagatatcgccatctgtattgaagaagcctaatgagtttacgctattagaaagacgctaatctcgaataacaaataacaaatagttgcgccgccttggctatccacatcgcacagggcggcactgataacatgagaacacctatgactagccatactctagatcaattcaatagcaataacgaaaaactcagcccttggccgtggcaagtcaacgatgcagagctgtgctttgatatcgacgcattaggcaaaaaactcaaagatttaagccaagcctgttacttagtgaatcacagtgaaaaaggcttaggcatagcgcaaacggcagaagtgaccaccagtgacagccaagcgccattgggctcacaccctgtcagcgcctttgcccctgcccttggcacccaaagtttaggcgacagtaattttcgccgcgtacacggggttaaatacgcttactacgctggcgctatggctaacggtattgcctcagaagaactggttatcgcgctgggccaagcgggcattttgtgctcatttggcgcggcggggttaattccatcccgcgttgaagcggccattacccgcattcaagcggcgctgcctaatggtccttacgcttttaacttaattcacagcccaagtgagcccgcattagagcgcggcagcgttgagctgtttttaaaacataaagtgcgcacggtcgaagcctcggcatttttaggcttaacgccgcaaatcgtctattaccgcgcagcgggtttgagccgcgacgaacatggcgagatagtcattggcaacaaagtcatagccaaaatcagccgcaccgaagtcgcgactaagtttatggagccggcgccggccaaaatcctccagcaattagtgagtgacggccttatcagccaagaccaaatggcgatggcattactggtgcccatggcagacgacatcacagccgaagccgactccggcggccataccgacaatcgtccactcgtcacgctattgccgacgattttggcgcttaaagatgaaatccaagctaagtatcaatacaaaacgcctatccgcgtgggtgctggcggcggcgtcggtacacccgatgcagcattagccaccttcaacatgggcgcggcgtttatcgtcacaggctcaatcaaccaagcctgcgtcgaagcgggcgccagcgaacacacccgtaagttactcgccaccacagaaatggccgatgtgactatggcgcctgccgccgatatgttcgaaatgggcgtgaaattacaagtggttaagcgcggcaccctgttcccaatgcgcgccaataagctttatgaaatctacacccgttacgattcgattgatgccatccctgcggacgagcgtaaaaaactcgaagagcaagtgttccgctcatcactcgatgacatttggactggcacagtagcccactttaacgagcgcgatcctaagcaaatcgagcgcgcactggataaccccaaacgtaagatggcgctgattttccgctggtatttgggtctatcgagccgctggtcaaacacaggcgaagtcggccgcgaaatggattaccaaatctgggctggcccagcgctcggcgcatttaacgcttgggcaaaaggcagttatttagatgactataaggcccgtaatgcggtggatttagccaaacatttaatggtgggcgcagcatatcaatcccgcattaacctgctgttatcccaaggcgcgagcattccagttagcctgcagcgctggaaaccgataaatcgtttttaacccctactggacaatagatacatcaagtagggactcaactccctgcttgatgcag

SEQ ID NO: 2 - DNA, 20 bp, synthetic construct
  misc_feature 1..20: note = nucleic acid sequence of 16S rRNA primer 8F
  source 1..20: mol_type = other DNA
agagtttgatcmtggctcag

SEQ ID NO: 3 - DNA, 19 bp, synthetic construct
  misc_feature 1..19: note = nucleic acid sequence of 16S rRNA primer 1492R
  source 1..19: mol_type = other DNA
ggmtaccttgttacgactt

SEQ ID NO: 4 - RNA, 1438 nt, synthetic construct
  misc_feature 1..1438: note = nucleic acid sequence of 16S rRNA of OR1 strain
  source 1..1438: mol_type = other RNA
tcagatgaacgctggcggcaggcctaacacatgcaagtcgagcggtagagagaagcttgcttctcttgagagcggcggacgggtgagtaatgcctaggaatctgcctggtagtgggggataacgttcggaaacgaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggtacca

SEQ ID NO: 5 - RNA, 767 nt, synthetic construct
  misc_feature 1..767: note = nucleic acid sequence of 16S rRNA of OR2 strain
  source 1..767: mol_type = other RNA
gtaagcttcgacctcagtgtcagtattagtccaggtggtcgccttcgccactggtgttccttcctatatctacgcatttcaccgctacacaggaaattccaccaccctctaccatactctagtcagtcagttttgaatgcagttcccaggttgagcccggggatttcacatccaacttaacaaaccacctacgcgcgctttacgcccagtaattccgattaacgcttgcaccctctgtattaccgcggctgctggcacagagttagccggtgcttattctgtcggtaacgtcaaaacaatcacgtattaggtaactgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgttcgtttccgaacgttatcccccactaccaggcagattcctaggcattactcacccgtccgccgctctcaagagaagcaagcttctctctaccgctcgacttgcatgtgttaggcctgccgccagcgtcatctggaagtcaaaaacccctcttttttgggc

SEQ ID NO: 6 - RNA, 1464 nt, synthetic construct
  misc_feature 1..1464: note = nucleic acid sequence of 16S rRNA of OR3 strain
  source 1..1464: mol_type = other RNA
gcaatcccgtggtaccgtcctcccgaagttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccattacgtgctggtaactaaggacaagggttgcgctcgttacgggacttaacccaacatctcacgacacgagctgacgacagccatgcagcacctgtctcaatgttcccgaaggcaccaatctatctctagaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaaaagctcaaggcttccaacggctagttgacatcgtttacggcgtggactaccagggtatctaatcctgtttgctccccacgctttctcacctcactgtcagtattaatccaggtggtcgccttcgccactggtgttccttcctatatctacgcatttcaccgctacacaggaaattccaccaccctctaccatactctagtcagtcagttttgaatgcagttcccaggttgagcccggggatttcacatccaacttaacaaaccacctacgcgcgctttacgcccagtaattccgattaacgcttgcaccctctgtattaccgcggctgctggcacagagttagccggtgcttattctgtcggtaacgtcaaaacactaacgtattaagttaatgcccttcctcccaacttaaagtgctttacaatccgaagaccttcttcacacacgcggcatggctggatcaggctttcgcccattgtccaatattccccactgctgcctcccgtaggagtctggaccgtgtctcacttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgttcgtttccgaacgttatcccccactaccaggcagattcctaggcattactcacccgtccgccgctctcaagagaagcaagcttctctctaccgctcgacttgcatgtgttaggcctgccgcacgtttatctggattttcaataaaaaaaaagg

SEQ ID NO: 7 - RNA, 1431 nt, synthetic construct
  misc_feature 1..1431: note = nucleic acid sequence of 16S rRNA of OR4 strain
  source 1..1431: mol_type = other RNA
cgacggctccctccacaagggttaggccaccggcttcaggtgttaccgactttcatgacttgacgggcggtgtgtacaagacccgggaacgtattcaccgcagcgttgctgatctgcgattactagcgactccgacttcatgaggtcgagttgcagacctcaatccgaactgggaccggctttttgggattcgctccacctcgcggtattgcagccctttgtaccggccattgtagcatgcgtgaagcccaagacataaggggcatgatgatttgacgtcatccccaccttcctccgagttgaccccggcagtatcccatgagttcccaccattacgtgctggcaacatagaacgagggttgcgctcgttgcgggacttaacccaacatctcacgacacgagctgacgacaaccatgcaccacctgtttacgagtgtccaaagagttgaccatttctggcccgttctcgtatatgtcaagccttggtaaggttcttcgcgttgcatcgaattaatccgcatgctccgccgcttgtgcgggtccccgtcaattcctttgagttttagccttgcggccgtactccccaggcggggaacttaatgcgttagctgcgtcacggaatccgtggaatggaccccacaactagttcccaacgtttacggggtggactaccagggtatctaagcctgtttgctccccaccctttcgctcctcagcgtcagttacggcccagagatctgccttcgccatcggtgttcctcctgatatctgcgcattccaccgctacaccaggaattccaatctcccctaccgcactctagtctgcccgtacccactgcaggccggaggttgagcctccggatttcacagcagacgcgacaaaccgcctacgagctctttacgcccaataattccggataacgcttgcgccctacgtattaccgcggctgctggcacgtagttagccggcgctttttctgcaggtaccgtcactttcgcttcttccctgctaaaagaggtttacaacccgaaggccgtcgtccctcacgcggcgttgctgcatcaggcttgcgcccattgtgcaatattccccactgctgcctcccgtaggagtctgggccgtgtctcagtcccagtgtggccggtcaccctctcaggccggctacccgtcgacgccttggtgagccattacctcaccaacaagctgataggccgcgagcccatccccaaccgaaattctttccagacgcagaccatgcggtcacgtcacatatccagtattagacgccgtttccagcgcttatcccagagtcaggggcaggttgctcacgtgttactcacccgttcgccactgatcccacagagcaagctccgtgttcaccgttcgacttgcatgtgttaagcacgccgccagcgttcatcctgagcctg

SEQ ID NO: 8 - RNA, 1443 nt, synthetic construct
  misc_feature 1..1443: note = nucleic acid sequence of 16S rRNA of OR5 strain
  source 1..1443: mol_type = other RNA
tggtaacgccctcccgaaggttaagctatctacttctggtgcaacccactcccatggtgtgacgggcggtgtgtacaaggcccgggaacgtattcaccgcaacattctgatttgcgattactagcgattccgacttcacggagtcgagttgcagactccgatccggactacgacgcgctttttgggattcgctcactatcgctagcttgcagccctctgtacgcgccattgtagcacgtgtgtagccctggccgtaagggccatgatgacttgacgtcatccccaccttcctccggtttatcaccggcagtctcccttgagttcccaccattacgtgctggcaacaaaggacaggggttgcgctcgttgcgggacttaacccaacatctcacgacacgagctgacgacagccatgcagcacctgtgttctgattcccgaaggcactcccgcatctctgcaggattccagacatgtcaaggccaggtaaggttcttcgcgttgcatcgaattaaaccacatgctccaccgcttgtgcgggcccccgtcaattcatttgagttttaaccttgcggccgtactccccaggcggtcgatttaacgcgttagctccggaagccacgtctcaaggacacagcctccaaatcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagctggacagttttaaatgcaattcccaggttgagcccggggctttcacatctaacttatccaaccgcctgcgtgcgctttacgcccagtaattccgattaacgcttgcaccctccgtattaccgcggctgctggcacggagttagccggtgcttcttctgcgagtaacgtcacagctggtaggtattagctaccaacctttcctcctcgctgaaagtgctttacaacccgaaggccttcttcacacacgcggcatggctgcatcagggtttcccccattgtgcaatattccccactgctgcctcccgtaggagtctggaccgtgtctcagttccagtgtggctgatcatcctctcagaccagctagggatcgtcgccttggtgagccattacctcaccaactagctaatcccacctgggttcatccaatcgcgcaaggcccgaaggtcccctgctttcccccgtagggcgtatgcggtattagctaccgtttccagtagttatccccctcgactgggcagatccccaggcattactcacccgtccgccgctcgccggcaaaagtagcaagctactttcccgctgccgctcgacttgcatgtgttaggcctgccgccagcgttcatc

SEQ ID NO: 9 - RNA, 757 nt, synthetic construct
  misc_feature 1..757: note = nucleic acid sequence of 16S rRNA of OR6 strain
  source 1..757: mol_type = other RNA
gtagcttcgctctcagcgtcagtaacggcccagagatctgccttcgccatcggtgttcttcctgatatctgcgcattccaccgctacaccaggagttccaatctcccctaccgcactctagtctgcccgtacccactgcaggcccgaggttgagcctcgggttttcacagcagacgcgacagaccgcctacgagctctttacgcccaataattccggacaacgcttgccccctacgtattaccgcggctgctggcacgtagttagccggagctttttctgcaggtaccgtcactttcgcttcttccctactaaaagaggtttacaacccgaaggccgtcatccctcacgcggcgttgctgcatcaggcttgcgcccattgtgcaatattccccactgctgcctcccgtaggagtctgggccgtgtctcagtcccagtgtggccggtcaccctctcaggccggctacccgtcgtcgccttggtgggccgttacctcaccaactagctgataggccgcgagctcatcccgaaccgaagttctttccacgcgcagaagatgccttcgcgcgtcgtatccggtattagacgtcgtttccaacgcttatcccagagtccggggcagattgctcacgtgttactcacccgttcgccactgatccggaggagcaagctcctccttcacagttcgacttgcatgtgttaagcacgccgccagcgttcgtcctgagctgagaacccaacatactcaatctgagcag

SEQ ID NO: 10 - RNA, 1395 nt, synthetic construct
  misc_feature 1..1395: note = nucleic acid sequence of 16S rRNA of OR7 strain
  source 1..1395: mol_type = other RNA
cgtggtcgcctgcctccttgcggttagcgcgacgccttcgggtaaacccaactcccatggtgtgacgggcggtgtgtacaaggcccgggaacgtattcaccgtggcatgctgatccacgattactagcgattccaccttcatgcactcgagttgcagagtgcaatctgaactgagacggctttttgggattagctcgaggtcaccctttcgctgcccattgtcaccgccattgtagcacgtgtgtagcccagcctgtaagggccatgaggacttgacgtcatccccaccttcctcgcggcttatcaccggcagtccccctagagttcccaactgaatgatggcaactaggggcgagggttgcgctcgttgcgggacttaacccaacatctcacgacacgagctgacgacagccatgcagcacctgtgttccggccagccgaactgaagaaaggcatctctgccgatcaaaccggacatgtcaaaagctggtaaggttctgcgcgttgcttcgaattaaaccacatgctccaccgcttgtgcgggcccccgtcaattcctttgagttttaatcttgcgaccgtactccccaggcggaatgcttaaagcgttagctgcgccactgaagagcaagctccccaacggctggcattcatcgtttacggcgtggactaccagggtatctaatcctgtttgctccccacgctttcgcgcctcagcgtcagtttcggaccagttggccgccttcgccaccggtgttcttgcgaatatctacgaatttcacctctacactcgcagttccaccaacctcttccgaactcaagactcccagtatcgaaggcaattccagggttgagccctgggctttcacctccgacttaaaagtccgcctacgcgccctttacgcccagtgattccgagcaacgctagcccccttcgtattaccgcggctgctggcacgaagttagccggggcttattcttccggtacagtcattatcttcccggacaaaagagctttacaaccctaaggccttcatcactcacgcggcatggctggatcaggctttcgcccattgtccaatattccccactgctgcctcccgtaggagtctgggccgtgtctcagtcccagtgtggctgatcatcctctcagaccagctactgatcgtcgccttggtgagcctttacctcaccaactagctaatcagacgcgggccgatctttcggcgataaatctttcccccgaagggcgtatccggtattagtccaagtttccctggattattccgaaccgaaaggcacgttcccacgcgttactcacccgtctgccactagcaccgaagtgcccgttcgacttgcatgtgttaagcctgccgccagcgttcgctctgagca

SEQ ID NO: 11 - RNA, 771 nt, synthetic construct
  misc_feature 1..771: note = nucleic acid sequence of 16S rRNA of OR8 strain
  source 1..771: mol_type = other RNA
ggtaggctttcgactgagcgtcagtctttgtccagggggccgccttcgccaccggtattcctccagatctctacgcatttcaccgctacacctggaattctacccccctctacaagactctagctggacagttttaaatgcaattcccaggttgagcccggggctttcacatctaacttatccaaccgcctgcgtgcgctttacgcccagtaattccgattaacgcttgcaccctccgtattaccgcggctgctggcacggagttagccggtgcttcttctgcgagtaacgtcacagctgataggtattagctatcaacctttcctcctcgctgaaagtgctttacaacccgaaggccttcttcacacacgcggcatggctgcatcagggtttcccccattgtgcaatattccccactgctgcctcccgtaggagtctggaccgtgtctcagttccagtgtggctgatcatcctctcagaccagctagggatcgtcgccttggtgagccattacctcaccaactagctaatcccacctgggttcatccaatcgcgcaaggcccgaaggtcccctgctttcccccgtagggcgtatgcggtattagctaccgtttccagtagttatccccctcgactgggcagatccccaggcattactcacccgtccgccgctcgccggcaaaagtagcaagctactttcccgctgccgctcgacttgcatgtgttagctgcccgccagcgttcatctgacgaataaaaaaaaacatataa

SEQ ID NO: 12 - RNA, 1442 nt, synthetic construct
  misc_feature 1..1442: note = nucleic acid sequence of 16S rRNA of OR12 strain
  source 1..1442: mol_type = other RNA
ggtaccgtcctcccgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatccatctctggaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagagctcaaggctcccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagcttgccagttttggatgcagttcccaggttgagcccggggatttcacatccaacttaacaaaccacctacgcgcgctttacgcccagtaattccgattaacgcttgcaccctctgtattaccgcggctgctggcacagagttagccggtgcttattctgtcggtaacgtcaaaattgcagagtattaatctacaacccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgttcctttcgaaacgttgtcccccactaccaggcagattcctaggcattactcacccgtccgccgctgaatccaggagcaagctcctctcatccgctcgacttgcatgtgttaggcctgccgccagcgttcaatctgagcag

SEQ ID NO: 13 - RNA, 1444 nt, synthetic construct
  misc_feature 1..1444: note = nucleic acid sequence of 16S rRNA of OR13 strain
  source 1..1444: mol_type = other RNA
tggtaacgccctcccgaaggttaagctatctacttctggtgcaacccactcccatggtgtgacgggcggtgtgtacaaggcccgggaacgtattcaccgcaacattctgatttgcgattactagcgattccgacttcacggagtcgagttgcagactccgatccggactacgacgcgctttttgggattcgctcactatcgctagcttgcagccctctgtacgcgccattgtagcacgtgtgtagccctggccgtaagggccatgatgacttgacgtcatccccaccttcctccggtttatcaccggcagtctcccttgagttcccaccattacgtgctggcaacaaaggacaggggttgcgctcgttgcgggacttaacccaacatctcacgacacgagctgacgacagccatgcagcacctgtgttctgattcccgaaggcactcccgcatctctacaggattccagacatgtcaaggccaggtaaggttcttcgcgttgcatcgaattaaaccacatgctccaccgcttgtgcgggcccccgtcaattcatttgagttttaaccttgcggccgtactccccaggcggtcgatttaacgcgttagctccggaagccacgtctcaaggacacagcctccaaatcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagctggacagttttaaatgcaattcccaggttgagcccggggctttcacatctaacttatccaaccgcctgcgtgcgctttacgcccagtaattccgattaacgcttgcaccctccgtattaccgcggctgctggcacggagttagccggtgcttcttctgcgagtaacgtcacagctgatgcgtattaggcatcaacctttcctcctcgctgaaagtgctttacaacccgaaggccttcttcacacacgcggcatggctgcatcagggtttcccccattgtgcaatattccccactgctgcctcccgtaggagtctggaccgtgtctcagttccagtgtggctgatcatcctctcagaccagctagggatcgtcgccttggtgagccattacctcaccaacaagctaatcccacctgggcatatccaatcgcgcaaggcccgaaggtcccctgctttcccccgtagggcgtatgcggtattagcagtcgtttccaactgttatccccctcgactgggcaatttcccaggcattactcacccgtccgccgctcgccggcaaaagtagcaagctactttcccgctgccgctcgacttgcatgtgttaggcctgccgccagcgttcatct

SEQ ID NO: 14 - RNA, 1438 nt, synthetic construct
  misc_feature 1..1438: note = nucleic acid sequence of 16S rRNA of OR14 strain
  source 1..1438: mol_type = other RNA
tggtaccgtcctcccgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccattacgtgctggtaactaaggacaagggttgcgctcgttacgggacttaacccaacatctcacgacacgagctgacgacagccatgcagcacctgtctcaatgttcccgaaggcaccaatctatctctagaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaaaagctcaaggcttccaacggctagttgacatcgtttacggcgtggactaccagggtatctaatcctgtttgctccccacgctttcgcacctcagtgtcagtattagtccaggtggtcgccttcgccactggtgttccttcctatatctacgcatttcaccgctacacaggaaattccaccaccctctaccatactctagtcagtcagttttgaatgcagttcccaggttgagcccggggatttcacatccaacttaacaaaccacctacgcgcgctttacgcccagtaattccgattaacgcttgcaccctctgtattaccgcggctgctggcacagagttagccggtgcttattctgtcggtaacgtcaaaacactaacgtattaggttaatgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgttcgtttccgaacgttatcccccactaccaggcagattcctaggcattactcacccgtccgccgctctcaagagaagcaagcttctctctaccgctcgacttgcatgtgttaggcctgccgccagcgttcatctgg

SEQ ID NO: 15 - RNA, 1448 nt, synthetic construct
  misc_feature 1..1448: note = nucleic acid sequence of 16S rRNA of GI12 strain
  source 1..1448: mol_type = other RNA
atgttcacttaggcggctggctccaaaaggttacctcaccgacttcgggtgttacaaactctcgtggtgtgacgggcggtgtgtacaaggcccgggaacgtattcaccgcggcatgctgatccgcgattactagcgattccagcttcatgtaggcgagttgcagcctacaatccgaactgagaacggttttatgggattggctaaacctcgcggtcttgcagccctttgtaccgtccattgtagcacgtgtgtagcccaggtcataaggggcatgatgatttgacgtcatccccaccttcctccggtttgtcaccggcagtcatcttagagtgcccaactgaatgctggcaactaagatcaagggttgcgctcgttgcgggacttaacccaacatctcacgacacgagctgacgacaaccatgcaccacctgtcactctgtcccccgaaggggaaagccctatctctagggttgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagtgtcagttacagaccagaaagtcgccttcgccactggtgttcctccaaatatctacgcatttcaccgctacacttggaattccactttcctcttctgcactcaagttccccagtttccaatgaccctccacggttgagccgtgggctttcacatcagacttaagaaaccacctgcgcgcgctttacgcccaataattccggacaacgcttgccacctacgtattaccgcggctgctggcacgtagttagccgtggctttctggttaggtaccgtcaaggcgccgccctattcgaacggcacttgttcttccctaacaacagagttttacgatccgaaaaccttcttcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccattacctcaccaactagctaatgcgccgcgggtccatctgtaagtgatagccgaagccacctttcaaccttcccccatgcgggggtaggtgttatccggtattagccccggtttcccggagttatcccagtcttacaggcaggttacccacgtgttactcacccgtccgccgctgatctcagggagcaagctcccatcgatccgctcgacttgcatgtatagcacccgcact

SEQ ID NO: 16 - RNA, 1475 nt, synthetic construct
  misc_feature 1..1475: note = nucleic acid sequence of 16S rRNA of GI431 strain
  source 1..1475: mol_type = other RNA
agaatcccgtggtaacgccctcccgaaggttaagctatctacttctggtgcaacccactcccatggtgtgacgggcggtgtgtacaaggcccgggaacgtattcaccgcaacattctgatttgcgattactagcgattccgacttcacggagtcgagttgcagactccgatccggactacgacgcgctttttgggattcgctcactatcgctagcttgcagccctctgtacgcgccattgtagcacgtgtgtagccctggccgtaagggccatgatgacttgacgtcatccccaccttcctccggtttatcaccggcagtctcccttgagttcccaccattacgtgctggcaacaaaggacaggggttgcgctcgttgcgggacttaacccaacatctcacgacacgagctgacgacagccatgcagcacctgtgttctgattcccgaaggcactcccgcatctctgcaggattccagacatgtcaaggccaggtaaggttcttcgcgttgcatcgaattaaaccacatgctccaccgcttgtgcgggcccccgtcaattcatttgagttttaaccttgcggccgtactccccaggcggtcgatttaacgcgttagctccggaagccacgtctcaaggacacagcctccaaatcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagctggacagttttaaatgcaattcccaggttgagcccggggctttcacatctaacttatccaaccgcctgcgtgcgctttacgcccagtaattccgattaacgcttgcaccctccgtattaccgcggctgctggcacggagttagccggtgcttcttctgcgagtaacgtcacagctggcagttattagctaccaacctttcctcctcgctgaaagtgctttacaacccgaaggccttcttcacacacgcggcatggctgcatcagggtttcccccattgtgcaatattccccactgctgcctcccgtaggagtctggaccgtgtctcagttccagtgtggctgatcatcctctcagaccagctagggatcgtcgccttggtgagccattacctcaccaactagctaatcccacctgggttcatccaatcgcgcaaggcccgaaggtcccctgctttcccccgtagggcgtatgcggtattagctaccgtttccagtagttatccccctcgactgggcagatccccaggcattactcacccgtccgccgctcgccggcaaaagtagcaagctactttcccgctgccgctcgactgcatgtgtagcgccgcctgcaccgctacacctggtaattctacccccctctacag

SEQ ID NO: 17 - RNA, 1438 nt, synthetic construct
  misc_feature 1..1438: note = nucleic acid sequence of 16S rRNA of GI71 strain
  source 1..1438: mol_type = other RNA
catccaaaacccgtggtgagcgccatcctaaaaggttaggctacccacttctggtgcaatcaactcccatggtgtgacgggcggtgtgtacaaggcccgggaacgtattcaccgcggcattctgatccgcgattactagcgattcctacttcatggagtcgagttgcagactccaatctggactacgataggctttttgagattcgcatcacatcgctgtgtagctgccctctgtacctaccattgtagcacgtgtgtagccctggtcgtaagggccatgatgacttgacgtcgtccccgccttcctccagtttgtcactggcagtatccttagagttcccggccaaaccgctggtaactaaggacaagggttgcgctcgttgcgggacttaacccaacatctcacgacacgagctgacgacagccatgcagcacctgtattctaattcccgaaggcactcccgcatctctgcaggattctagatatgtcaagaccaggtaaggttcttcgcgttgcatcgaattaaaccacatgctccaccgcttgtgcgggcccccgtcaattcatttgagttttaaccttgcggccgtactccccaggcggtctacttattgcgttagctgcgtcactaagtcctcaagggacccaacgactagtagacatcgtttacggcgtggactaccagggtatctaatcctgtttgctacccacgctttcgaacctcagtgtcagtatgatgccagaaggctgccttcgccatcggtattcctccagatctctacgcatttcaccgctacacctggaattctaccttcctctcacctactctagcctaacagtatcagatgccgttcccaggttaagcccggggctttcacatctgacttatcaagccacctacgctcgctttacgcccagtaattccgattaacgcttgcaccctctgtattaccgcggctgctggcacagagttagccggtgcttattctgcagctaatgtcatcgtccatgggtattaaccatggagtcttcttcactgcttaaagtgctttacaaccaaaaggccttcttcacacacgcggcatggctggatcagggttgcccccattgtccaatattccccactgctgcctcccgtaggagtccgggccgtgtctcagtcccggtgtggctgatcatcctctcagaccagctacagatcgtcgccatggtaggcctttaccccaccatctagctaatccgacttaggctcatctaatagcgagagcaacaagttgccccctttctcccgtaggtcgtatgcggtattaattcgagtttccccgagctatcccccactactaggtagattcctaagtattactcacccgtccgccgctcgtcagcgagaagcaagcttctcctgttaccgctcgacttgcatggtaagctcgcccg

SEQ ID NO: 18 - RNA, 820 nt, synthetic construct
  misc_feature 1..820: note = nucleic acid sequence of 16S rRNA of GI83 strain
  source 1..820: mol_type = other RNA
acaaaacccgtggtgatcgccttccttacggttaggctaaccacttctggtgcaatcaactcccatggtgtgacgggcggtgtgtacaaggcccgggaacgtattcaccgcggcattctgatccgcgattactagcgattcctacttcatggagtcgagttgcagactccaatctggactacgataggctttttgagattcgcatcacatcgctgtgtagctgccctctgtacctaccattgtagcacgtgtgtagccctggtcgtaagggccatgatgacttgacgtcgtccccgccttcctccagtttgtcactggcagtatccttagagttcccggcttaacccgctggtaactaaggacaagggttgcgctcgttgcgggacttaacccaacatctcacgacacgagctgacgacagccatgcagcacctgtattctaattcccgaaggcactcccgcatctctgcaggattctagatatgtcaagaccaggtaaggttcttcgcgttgcatcgaattaaaccacatgctccaccgcttgtgcgggcccccgtcaattcatttgagttttaaccttgcggccgtactccccaggcggtctacttattgcgttagctgcgtcactaagtcctcaagggacccaacgactagtagacatcgtttacggcgtggactaccagggtatctaatcctgtttgctacccacgctttcgagcctcagtgtcagtatgatgccagaaggctgccttcgccatcggtattcctccagatctctacgcatttcaccgctacacctggaattctacccttcctctca

SEQ ID NO: 19 - RNA, 1382 nt, synthetic construct
  misc_feature 1..1382: note = nucleic acid sequence of 16S rRNA of KME43 strain
  source 1..1382: mol_type = other RNA
agagacgacgctggcggcgtgcttaatacatgcaagtcgagcggaccgacgggagcttgctcccttaggtcagcggcggacgggtgagtaacacgtgggtaacctgcctgtaagactgggataactccgggaaaccggggctaataccggatgcttgattgaaccgcatggttcaatcataaaaggtggcttttagctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattcacgtgttagcggtgaaatgcgtagagatgtggaggaacaccagtggcgaaggcgactctctggtctgtaactgacgctgaggcgcgaaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacaca

SEQ ID NO: 20 - RNA, 1436 nt, synthetic construct
  misc_feature 1..1436: note = nucleic acid sequence of 16S rRNA of SI20 strain
  source 1..1436: mol_type = other RNA
ctcaggatgaacgctggcggcgtgcttaacacatgcaagtcgaacggtgaacacggagcttgctctgtgggatcagtggcgaacgggtgagtaacacgtgagcaacctgcccctgactctgggataagcgctggaaacggcgtctaatactggatatgtgacgtgaccgcatggtctgcgtctggaaagaatttcggttggggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgcaagcctgatgcagcaacgccgcgtgagggacgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatccggaggctcaacctccggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtccattccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgcgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtttagtcttgaacctcggcccacctgatgcaactagggttctagtgggtcccaggagccatttaataactcc

SEQ ID NO: 21 - RNA, 1463 nt, synthetic construct
  misc_feature 1..1463: note = nucleic acid sequence of 16S rRNA of SI26 strain
  source 1..1463: mol_type = other RNA
tgctcaggacgaacgctggcggcgtgcctaatacatgcaagtcgagcgatgattgaagatagcttgctattttcatgaagagcggcgaacgggtgagtaacgcgtgggaaatctgccgagtagcgggggacaacgtttggaaacgaacgctaataccgcataacaatgagaatcgcatgattcttatttgaaagaagcaattgcttcactacttgatgatcccgcgttgtattagctagttggtagtgtaaaggactaccaaggcgatgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaaactctgttgttagagaagaacgttaagtagagtggaaaattacttaagtgacggtatctaaccagaaagggacggctaactacgtgccagcagccgcggtaatacgtaggtcccaagcgttgtccggatttattgggcgtaaagcgagcgcaggtggtttcttaagtctgatgtaaaaggcagtggctcaaccattgtgtgcattggaaactgggagacttgagtgcaggagaggagagtggaattccatgtgtagcggtgaaatgcgtagatatatggaggaacaccggaggcgaaagcggctctctggcctgtaactgacactgaggctcgaaagcgtggggagcaaacaggattagataccctggtagtccacgccgtaaacgatgagtgctagctgtagggagctataagttctctgtagcgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctatccttagagataaggagttccttcgggacacgggatacaggtggtgcatggttgtcgtcagctcgtgtcgtgagatgttgggttaagtcccgcaacgagcgcaacccttattactagttgccatcattaagttgggcactctagtgagactgccggtgataaaccggaggaaggtggggatgacgtcaaatcatcatgccccttatgacctgggctacacacgtgctacaatggatggtacaacgagtcgccaacccgcgagggtgcgctaatctcttaaaaccattctcagttcggattgcaggctgcaactcgcctgcatgaagtcggaatcgttagtaatcgcggatcagcacgccgcggtgaaaacaggacccgacattgtacaccgctccctttaagtcgtaccaggtccaggtcaattcgtaccaaggatcctagagtttcccacgatcctaaagtttgctg

SEQ ID NO: 22 - RNA, 1339 nt, synthetic construct
  misc_feature 1..1339: note = nucleic acid sequence of 16S rRNA of Squ08 strain
  source 1..1339: mol_type = other RNA
ctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgccgaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgttcagatatcaggaggaacaccgatggcggaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgtt

(19) United States
(12) Patent Application Publication
(10) Pub. No.: US 2025/0249047 A1
(43) Pub. Date: Aug. 7, 2025

(54) NOVEL ORGANISM BREEDING TECHNIQUE

(71) Applicant: HOLO BIO CO., LTD., Kyoto-shi, Kyoto (JP)

(72) Inventors: Masato UMEDA, Kyoto (JP); Naoto JUNI, Kyoto (JP); Makoto YAMASHITA, Kyoto (JP)

(73) Assignee: HOLO BIO CO., LTD., Kyoto-shi, Kyoto (JP)

(21) Appl. No.: 18/730,653

(22) PCT Filed: Jan. 19, 2023
(86) PCT No.: PCT/JP2023/001513

§ 371 Date: Apr. 17, 2025

(30) Foreign Application Priority Data
Jan. 20, 2022 (JP) 2022-007459

(51) Int. Cl. A61K 35/741; C12N 1/20; C12Q 1/689

(52) U.S. Cl. CPC A61K 35/741; C12N 1/20; C12Q 1/689; C12Q 2600/136

(57) ABSTRACT

The present disclosure provides a method for producing an improved target organism, the method including: a step A) of selecting an individual exhibiting the improvement from candidate microorganisms of an organism species to which a derived individual different from the target individual belongs; a step B) of obtaining, in a derived individual exhibiting the improvement, an exogenous microorganism involved in the improvement or a part thereof from a gastrointestinal bacterial flora of the derived individual; a step C) of introducing the exogenous microorganism or a part thereof into the target individual; and a step D) of optionally confirming the properties of the gastrointestinal bacterial flora in the target individual, and confirming that a desired improvement is achieved.

### TECHNICAL FIELD

The present disclosure relates to a novel breeding method. The present disclosure relates to a breeding technique by changing the properties of a gastrointestinal tract (for example, an intestinal tract). The present disclosure also relates to a novel strain derived from a gastrointestinal tract and a novel use of the strain derived from the gastrointestinal tract.

### BACKGROUND ART

A function of an organism varies with the individual species, and the individual species provide different capabilities. It may be preferable that a function provided in one organism species is provided in another organism species.

When it is desired to provide such a new function, breeding has been traditionally performed, and recently, the function has been provided by genetic manipulation.

However, breeding takes time, and in the case of genetic manipulation, it cannot be said that only desired properties are imparted.

In the studies on probiotics, it is considered that it is almost impossible that ingested viable bacteria survive in the intestinal tract and persist continuously in the intestinal tract (Non Patent Literature 1).

### CITATION LIST

### Non Patent Literature

Non Patent Literature 1: Tomotari Mitsuoka, History and Evolution of Probiotics, Japanese Journal of Lactic Acid Bacteria 22, 26-37, 2011.

### SUMMARY OF INVENTION

### Solution to Problem

The present disclosure presents the following in order to create an innovative technique by discovering and elucidating functions of organisms.

### (Item 1)

A composition for use in improving a target individual of an organism belonging to an organism species having a gastrointestinal tract, the composition containing an exogenous microorganism derived from a gastrointestinal tract of a derived individual different from the target individual, or a part thereof.

### (Item 2)

The composition according to any one of the items, in which the improvement is achieved by introducing the exogenous microorganism or a part thereof into a gastrointestinal microflora of the target individual.

### (Item 3)

The composition according to any one of the items, in which the improvement is achieved by introducing, into the target individual, a feature that is not present in the target individual but is present in the derived individual.

### (Item 4)

The composition according to any one of the items, in which the improvement is achieved by introducing, into the target individual, the exogenous microorganism or a part thereof that provides a feature that is not present in the target individual but is present in the derived individual.

### (Item 5)

The composition according to any one of the items, in which the improvement includes at least one selected from the group consisting of modification of nutritional and energy metabolism and modification of immune function and anti-inflammatory and anti-infective function.

### (Item 6)

The composition according to any one of the items, in which the improvement includes at least one selected from the group consisting of modification of fatty acid metabolism and/or amino acid metabolism, introduction of essential nutrients (for example, essential fatty acids, essential amino acids, vitamins, and the like), growth promotion, infectious disease prevention, enhancement of immune response ability (for example, enhancement of production of a polyunsaturated fatty acid and a lipid mediator that is a metabolite of the polyunsaturated fatty acid in the gastrointestinal tract by an introduced microorganism strain), promotion of utilization of a non-nutrient or a poor nutrient as a nutrient including nutrient conversion of fibers of a photosynthetic organism such as a plant in an animal and introduction of a substance having a high α-amylase activity, modification of diathesis, modification of feeding habit, fiber degradation of a photosynthetic organism including a plant, and metabolism of a polyunsaturated fatty acid.

### (Item 7)

The composition according to any one of the items, in which the organism is a mammal, a bird, an amphibian, a reptile, fish, a cephalopod, an arthropod, a crustacean, a shellfish, or a wheel animal.

### (Item 8)

The composition according to any one of the items, in which the organism is an organism provided for food, clothing, fuel, pet, pharmaceutical manufacture, and/or ornamental use.

### (Item 9)

The composition according to any one of the items, in which a part of the exogenous microorganism contains an enzyme, a nucleic acid encoding an enzyme, a virus, or a metabolite contained in the exogenous microorganism.

### (Item 10)

A composition for producing a fatty acid other than eicosapentaenoic acid (EPA), the composition containing a microorganism having an ability equivalent to that of GI35 strain or GI35 strain, or a part thereof.

### (Item 11)

A composition for producing a polyunsaturated fatty acid other than eicosapentaenoic acid (EPA), the composition containing an unsaturated fatty acid synthase group (SEQ ID NO: 1) derived from GI35 strain or a synthase group having an ability equivalent to that of the unsaturated fatty acid synthase group.

### (Item 12)

The composition according to any one of the items, in which the synthase group contains an extract of the GI35 strain.

### (Item 13)

The composition according to any one of the items, in which the fatty acid other than EPA is selected from the group consisting of palmitoleic acid, oleic acid, linoleic acid, γ-linolenic acid, α-linolenic acid, stearidonic acid, dihomo-γ-linolenic acid, arachidonic acid, eicosatetraenoic acid (ETA), osbond acid, docosapentaenoic acid (DPA), and docosahexaenoic acid (DHA).

### (Item 14)

A method for producing an improved target organism, the method including:
a step A) of selecting an individual exhibiting the improvement from candidate microorganisms of an organism species to which a derived individual different from the target individual belongs;
a step B) of obtaining, in a derived individual exhibiting the improvement, an exogenous microorganism involved in the improvement or a part thereof from a gastrointestinal microflora of the derived individual;
a step C) of introducing the exogenous microorganism or a part thereof into the target individual; and
a step D) of optionally confirming the properties of the gastrointestinal microflora in the target individual, and confirming that a desired improvement is achieved.

### (Item 15)

The method according to any one of the items, in which the step C) includes a step of introducing the exogenous microorganism or a part thereof into the target individual at least at a part of a time during breeding of the target individual.

### (Item 16)

The method according to any one of the items, in which the step C) includes a step of introducing the exogenous microorganism or a part thereof into the target individual at least at a part of a time during breeding of the target individual, and breeding the target individual without administering the microorganism during the rest of the time.

### (Item 17)

The method according to any one of the items, in which the step C) includes a step of introducing the exogenous microorganism or a part thereof into the target individual at least at a part of a time during growing of the target individual.

### (Item 18)

The method according to any one of the items, in which the step C) includes a step of introducing the exogenous microorganism or a part thereof into the target individual at least at a part of a time during growing of the target individual, and breeding the target individual without administering the microorganism during the rest of the time.

### (Item 19)

The method according to any one of the items, further including a step D′) of optionally confirming that a desired improvement is achieved in the target individual.

### (Item 20)

The method according to any one of the items, further including:
a step B′) of selecting an exogenous microorganism suitable (“compatible”) for the target individual, or a part thereof; and
a step C′) of introducing the suitable exogenous microorganism or a part thereof into the target individual.

### (Item 21)

The method according to any one of the items, in which the gastrointestinal tract is an intestine.

### (Item 22)

The method according to any one of the items, in which the improvement includes at least one selected from the group consisting of modification of nutritional and energy metabolism and modification of immune function and anti-inflammatory and anti-infective function.

### (Item 23)

The method according to any one of the items, in which the improvement includes at least one selected from the group consisting of modification of fatty acid metabolism and/or amino acid metabolism, introduction of essential nutrients (for example, essential fatty acids, essential amino acids, vitamins, and the like), growth promotion, infectious disease prevention, enhancement of immune response ability (for example, enhancement of production of a polyunsaturated fatty acid and a lipid mediator that is a metabolite of the polyunsaturated fatty acid in the gastrointestinal tract by an introduced microorganism strain), promotion of utilization of a non-nutrient or a poor nutrient as a nutrient including nutrient conversion of fibers of a photosynthetic organism such as a plant in an animal and introduction of a substance having a high α-amylase activity, modification of diathesis, modification of feeding habit, fiber degradation of a photosynthetic organism including a plant, and metabolism of a polyunsaturated fatty acid.

### (Item 24)

The method according to any one of the items, in which the exogenous microorganism is present outside the target organism.

(Item 25) A method for producing an organism having improved or altered nutrient availability, the method including
a step of introducing, into a gastrointestinal microflora, a microorganism or enzyme that imparts a metabolic activity of using a component that is not a nutrient source for the organism as a nutrient source for the organism and/or improves the metabolic activity of the organism for the component that is a nutrient source for the organism.

### (Item 26)

The method according to any one of the items, in which the step of introducing the microorganism or enzyme that improves the metabolic activity of the organism into the gastrointestinal microflora includes a step of introducing the microorganism or enzyme that improves the metabolic activity of the organism into the organism at least at a part of a time during breeding of the organism.

### (Item 27)

The method according to any one of the items, in which the step of introducing the microorganism or enzyme that improves the metabolic activity of the organism into the gastrointestinal microflora includes a step of introducing the microorganism or enzyme that improves the metabolic activity of the organism into the organism at least at a part of a time during breeding of the organism, and breeding the organism without administering the microorganism during the rest of the time.

### (Item 28)

The method according to any one of the items, in which the step of introducing the microorganism or enzyme that improves the metabolic activity of the organism into the gastrointestinal microflora includes a step of introducing the microorganism or enzyme that improves the metabolic activity of the organism into the organism at least at a part of a time during growing of the organism.

### (Item 29)

The method according to any one of the items, in which the step of introducing the microorganism or enzyme that improves the metabolic activity of the organism into the gastrointestinal microflora includes a step of introducing the microorganism or enzyme that improves the metabolic activity of the organism into the organism at least at a part of a time during growing of the organism, and breeding the organism without administering the microorganism during the rest of the time.

### (Item 30)

The method according to any one of the items, in which the nutrition includes one or more selected from the group consisting of a fatty acid, a carbon source (carbohydrate), woody biomass (for example, cellulose, hemicellulose, and lignin), an amino acid, a vitamin, a carotenoid, and a mineral.

### (Item 31)

The method according to any one of the items, in which a substance that is not nutritionally available in the organism is allowed to be nutritionally available.

### (Item 32)

A method for producing a target individual of an animal modified to have a component of a photosynthetic organism that is not a nutrient source in the animal as a nutrient source in the animal, the method including:
a step A) of providing an exogenous microorganism having an ability to convert the component of the photosynthetic organism into a nutrient source in the animal, or a part thereof, and
a step B) of introducing the exogenous microorganism or a part thereof into the target individual.

### (Item 33)

The method according to any one of the items, in which the step B) includes a step of introducing the exogenous microorganism or a part thereof into the target individual at least at a part of a time during breeding of the target individual.

### (Item 34)

The method according to any one of the items, in which the step B) includes a step of introducing the exogenous microorganism or a part thereof into the target individual at least at a part of a time during breeding of the target individual, and breeding the target individual without administering the microorganism during the rest of the time.

### (Item 35)

The method according to any one of the items, in which the step B) includes a step of introducing the exogenous microorganism or a part thereof into the target individual at least at a part of a time during growing of the target individual.

### (Item 36)

The method according to any one of the items, in which the step B) includes a step of introducing the exogenous microorganism or a part thereof into the target individual at least at a part of a time during growing of the target individual, and breeding the target individual without administering the microorganism during the rest of the time.

### (Item 37)

The method according to any one of the items, in which the animal is a mammal, a bird, an amphibian, a reptile, fish, a cephalopod, an arthropod, a crustacean, a shellfish, or a wheel animal.

### (Item 38)

The method according to any one of the items, in which the photosynthetic organism includes a plant and an alga.

### (Item 39)

The method according to any one of the items, in which the photosynthetic organism is selected from the group consisting of a herbaceous plant, a woody plant, a blue-green alga, a green alga, and a microalga.

### (Item 40)

The method according to any one of the items, in which the photosynthetic organism is provided in a living state or a non-living state, or as a processed product.

### (Item 41)

The method according to any one of the items, in which the nutrition is selected from a fatty acid, a carbon source (carbohydrate), cellulose, hemicellulose, and lignin as woody biomass, an amino acid, a vitamin, a carotenoid, and a mineral.

### (Item 42)

The method according to any one of the items, in which the exogenous microorganism is capable of converting the nutrient source in a normal growth environment of the animal.

### (Item 43)

The method according to any one of the items, in which the exogenous microorganism is intestinal bacteria of Japanese rice fish, and the nutrition is a combination of cellulose, hemicellulose, and lignin.

### (Item 44)

A composition for use in a method for producing an improved target organism, the composition containing an exogenous microorganism or a part thereof,
in which the method includes:
a step A) of selecting an individual exhibiting the improvement from candidate microorganisms of an organism species to which a derived individual different from the target individual belongs;
a step B) of obtaining, in a derived individual exhibiting the improvement, an exogenous microorganism involved in the improvement or a part thereof from a gastrointestinal microflora of the derived individual;
a step C) of introducing the exogenous microorganism or a part thereof into the target individual; and
a step D) of optionally confirming the properties of the gastrointestinal microflora in the target individual, and confirming that a desired improvement is achieved.

### (Item 45)

The method according to any one of the items, in which the step C) includes a step of introducing the exogenous microorganism or a part thereof into the target individual at least at a part of a time during breeding of the target individual.

### (Item 46)

The method according to any one of the items, in which the step C) includes a step of introducing the exogenous microorganism or a part thereof into the target individual at least at a part of a time during breeding of the target individual, and breeding the target individual without administering the microorganism during the rest of the time.

### (Item 47)

The method according to any one of the items, in which the step C) includes a step of introducing the exogenous microorganism or a part thereof into the target individual at least at a part of a time during growing of the target individual.

### (Item 48)

The method according to any one of the items, in which the step C) includes a step of introducing the exogenous microorganism or a part thereof into the target individual at least at a part of a time during growing of the target individual, and breeding the target individual without administering the microorganism during the rest of the time.

### (Item 49)

An individual of an organism belonging to an organism species having a gastrointestinal tract, the individual containing an exogenous microorganism derived from a gastrointestinal tract of a derived individual different from the individual, or a part thereof.

### (Item 50)

The individual according to any one of the items, in which a microorganism bacterial flora in the gastrointestinal tract is different from a naturally occurring microorganism bacterial flora.

### (Item 51)

The individual according to any one of the items, in which in a microflora in the gastrointestinal tract, a diversity index of a metagenomic analysis result decreases, and a microflora contributing to digestion and absorption of nutrients increases.

### (Item 52)

A product produced by the individual according to any one of the items.

### (Item 53)

The product according to any one of the items, in which the product is selected from meat, an internal organ, milk, egg, and alcohol.

### (Item 54)

A processed product obtained by processing the product according to any one of the items.

### (Item 55)

The processed product according to any one of the items, in which the processed product is selected from a meat processed product or a dairy product.

### (Item 56)

A method for breeding the individual according to any one of the items.

### (Item 57)

The individual according to any one of the items, in which the individual is for use in the method according to any one of the items.

### (Item 58)

A method for producing a useful product for a human derived from a useful animal, the method including:
a step i) of providing an exogenous microorganism having an ability to convert a component of a photosynthetic organism that is not a nutrient source in the useful animal into a nutrient source in the useful animal, or a part thereof;
a step ii) of introducing the exogenous microorganism or a part thereof into the useful animal;
a step iii) of placing the useful animal under conditions under which the useful animal grows; and
a step iv) of optionally obtaining the useful product from the useful animal.

### (Item 59)

The method according to any one of the items, in which the step ii) includes a step of introducing the exogenous microorganism or a part thereof into the useful animal at least at a part of a time during breeding of the useful animal.

### (Item 60)

The method according to any one of the items, in which the step ii) includes a step of introducing the exogenous microorganism or a part thereof into the useful animal at least at a part of a time during breeding of the useful animal, and breeding the useful animal without administering the microorganism during the rest of the time.

### (Item 61)

The method according to any one of the items, in which the step ii) includes a step of introducing the exogenous microorganism or a part thereof into the useful animal at least at a part of a time during growing of the useful animal.

### (Item 62)

The method according to any one of the items, in which the step ii) includes a step of introducing the exogenous microorganism or a part thereof into the useful animal at least at a part of a time during growing of the useful animal, and breeding the useful animal without administering the microorganism during the rest of the time.

### (Item 63)

The method according to any one of the items, in which the useful product includes a product obtained directly from the useful animal.

### (Item 64)

The method according to any one of the items, in which the useful product includes a product obtained indirectly from the useful animal.

### (Item 65)

A composition for use in a method for producing a useful product for a human derived from a useful animal, the composition containing a microorganism derived from a gastrointestinal microflora or a part thereof, in which the method includes:
a step i) of providing an exogenous microorganism having an ability to convert a component of a photosynthetic organism that is not a nutrient source in the useful animal into a nutrient source in the useful animal, or a part thereof;
a step ii) of introducing the exogenous microorganism or a part thereof into the useful animal;
a step iii) of placing the useful animal under conditions under which the useful animal grows; and
a step iv) of optionally collecting the useful product from the useful animal, and
the exogenous microorganism is a microorganism derived from the gastrointestinal microflora.

### (Item 66)

The composition according to any one of the items, in which the step ii) includes a step of introducing the exogenous microorganism or a part thereof into the useful animal at least at a part of a time during breeding of the useful animal.

### (Item 67)

The composition according to any one of the items, in which the step ii) includes a step of introducing the exogenous microorganism or a part thereof into the useful animal at least at a part of a time during breeding of the useful animal, and breeding the useful animal without administering the microorganism during the rest of the time.

### (Item 68)

The composition according to any one of the items, in which the step ii) includes a step of introducing the exogenous microorganism or a part thereof into the useful animal at least at a part of a time during growing of the useful animal.

### (Item 69)

The composition according to any one of the items, in which the step ii) includes a step of introducing the exogenous microorganism or a part thereof into the useful animal at least at a part of a time during growing of the useful animal, and breeding the useful animal without administering the microorganism during the rest of the time.

### (Item 70)

An exogenous microorganism having an ability to convert a component of a photosynthetic organism that is not a nutrient source in a useful animal into a nutrient source in the useful animal, or a part thereof.

### (Item 71)

A microorganism derived from Japanese rice fish having an ability to degrade at least one selected from the group consisting of cellulose, hemicellulose, and lignin.

### (Item 72)

The microorganism according to any one of the items, in which the microorganism is at least one selected from the group consisting of the genus Pseudomonas, the genus Microbacterium, the genus Aeromonas, the genus Diaminobutyricmonas, the genus Bosea, the genus Shinella, and Fungi.

### (Item 73)

The microorganism according to any one of the items, in which the microorganism is at least one selected from the group consisting of Pseudomonas fluorescens, Pseudomonas extremorientalis, Microbacterium oxydans, Aeromonas veronii, Diaminobutyricmonas aerilata, Bosea robinae, Shinella curvata, Fungi, Pseudomons koreensis, and Aeromonas media.

### (Item 74)

The microorganism according to any one of the items, in which the microorganism is at least one selected from the group consisting of microorganisms of Pseudomonas sp. whose nucleic acid sequence of 16S rRNA is SEQ ID NO: 4, Pseudomonas sp. whose nucleic acid sequence of 16S rRNA is SEQ ID NO: 5, Pseudomonas sp. whose nucleic acid sequence of 16S rRNA is SEQ ID NO: 6, Microbacterium sp. whose nucleic acid sequence of 16S rRNA is SEQ ID NO: 7, Aeromonas sp. whose nucleic acid sequence of 16S rRNA is SEQ ID NO: 8, Diaminobutyricmonas sp. whose nucleic acid sequence of 16S rRNA is SEQ ID NO: 9, Bosea sp. whose nucleic acid sequence of 16S rRNA is SEQ ID NO: 10, Aeromonas sp. whose nucleic acid sequence of 16S rRNA is SEQ ID NO: 11, Pseudomons sp. whose nucleic acid sequence of 16S rRNA is SEQ ID NO: 12, Aeromonas sp. whose nucleic acid sequence of 16S rRNA is SEQ ID NO: 13, and Pseudomonas sp. whose nucleic acid sequence of 16S rRNA is SEQ ID NO: 14.

### (Item A1-1)

A method for improving a target individual of an organism belonging to an organism species having a gastrointestinal tract, the method including introducing, into the target individual, an exogenous microorganism derived from a gastrointestinal tract of a derived individual different from the target individual, or a part thereof.

### (Item A1-2)

The method according to any one of the items, in which the introduction includes introducing the exogenous microorganism or a part thereof into a gastrointestinal microflora of the target individual.

### (Item A1-3)

The method according to any one of the items, in which the introduction further includes introducing, into the target individual, a feature that is not present in the target individual but is present in the derived individual.

### (Item A1-4)

The method according to any one of the items, in which the introduction includes introducing, into the target individual, the exogenous microorganism or a part thereof that provides a feature that is not present in the target individual but is present in the derived individual.

### (Item A1-5)

The method according to any one of the items, in which the improvement includes at least one selected from the group consisting of modification of nutritional and energy metabolism and modification of immune function and anti-inflammatory and anti-infective function.

### (Item A1-6)

The method according to any one of the items, in which the improvement includes at least one selected from the group consisting of modification of fatty acid metabolism and/or amino acid metabolism, introduction of essential nutrients (for example, essential fatty acids, essential amino acids, vitamins, and the like), growth promotion, infectious disease prevention, enhancement of immune response ability (for example, enhancement of production of a polyunsaturated fatty acid and a lipid mediator that is a metabolite of the polyunsaturated fatty acid in the gastrointestinal tract by an introduced microorganism strain), promotion of utilization of a non-nutrient or a poor nutrient as a nutrient including nutrient conversion of fibers of a photosynthetic organism such as a plant in an animal and introduction of a substance having a high α-amylase activity, modification of diathesis, modification of feeding habit, fiber degradation of a photosynthetic organism including a plant, and metabolism of a polyunsaturated fatty acid.

### (Item A1-7)

The method according to any one of the items, in which the organism is a mammal, a bird, an amphibian, a reptile, fish, a cephalopod, an arthropod, a crustacean, a shellfish, or a wheel animal.

### (Item A1-8)

The method according to any one of the items, in which the organism is an organism provided for food, clothing, fuel, pet, pharmaceutical manufacture, and/or ornamental use.

### (Item A1-9)

The method according to any one of the items, in which a part of the exogenous microorganism contains an enzyme, a nucleic acid encoding an enzyme, a virus, or a metabolite contained in the exogenous microorganism.

### (Item A1-10)

A method for producing a fatty acid other than eicosapentaenoic acid (EPA), the method including: culturing a microorganism having an ability equivalent to that of GI35 strain or GI35 strain, or a part thereof; and recovering the fatty acid other than eicosapentaenoic acid (EPA) from the cultured microorganism.

### (Item A1-11)

A method for producing a fatty acid other than eicosapentaenoic acid (EPA), the method including incubating an unsaturated fatty acid synthase group (for example, SEQ ID NO: 1) derived from GI35 strain or a synthase group having an ability equivalent to that of the unsaturated fatty acid synthase group with a material of a polyunsaturated fatty acid other than eicosapentaenoic acid (EPA).

### (Item A1-12)

The method according to any one of the items, in which the synthase group contains an extract of the GI35 strain.

### (Item A1-13)

The method according to any one of the items, in which the fatty acid other than EPA is selected from the group consisting of palmitoleic acid, oleic acid, linoleic acid, γ-linolenic acid, α-linolenic acid, stearidonic acid, dihomo-γ-linolenic acid, arachidonic acid, eicosatetraenoic acid (ETA), osbond acid, docosapentaenoic acid (DPA), and docosahexaenoic acid (DHA).

### (Item A2-1)

A use of an exogenous microorganism derived from a gastrointestinal tract of a derived individual different from a target individual, or a part thereof for improvement of the target individual of an organism belonging to an organism species having a gastrointestinal tract.

### (Item A2-2)

The use according to any one of the items, in which the improvement is achieved by introducing the exogenous microorganism or a part thereof into a gastrointestinal microflora of the target individual.

### (Item A2-3)

The use according to any one of the items, in which the improvement is achieved by introducing, into the target individual, a feature that is not present in the target individual but is present in the derived individual.

### (Item A2-4)

The use according to any one of the items, in which the improvement is achieved by introducing, into the target individual, the exogenous microorganism or a part thereof that provides a feature that is not present in the target individual but is present in the derived individual.

### (Item A2-5)

The use according to any one of the items, in which the improvement includes at least one selected from the group consisting of modification of nutritional and energy metabolism and modification of immune function and anti-inflammatory and anti-infective function.

### (Item A2-6)

The use according to any one of the items, in which the improvement includes at least one selected from the group consisting of modification of fatty acid metabolism and/or amino acid metabolism, introduction of essential nutrients (for example, essential fatty acids, essential amino acids, vitamins, and the like), growth promotion, infectious disease prevention, enhancement of immune response ability (for example, enhancement of production of a polyunsaturated fatty acid and a lipid mediator that is a metabolite of the polyunsaturated fatty acid in the gastrointestinal tract by an introduced microorganism strain), promotion of utilization of a non-nutrient or a poor nutrient as a nutrient including nutrient conversion of fibers of a photosynthetic organism such as a plant in an animal and introduction of a substance having a high α-amylase activity, modification of diathesis, modification of feeding habit, fiber degradation of a photosynthetic organism including a plant, and metabolism of a polyunsaturated fatty acid.

### (Item A2-7)

The use according to any one of the items, in which the organism is a mammal, a bird, an amphibian, a reptile, fish, a cephalopod, an arthropod, a crustacean, a shellfish, or a wheel animal.

### (Item A2-8)

The use according to any one of the items, in which the organism is an organism provided for food, clothing, fuel, pet, pharmaceutical manufacture, and/or ornamental use.

### (Item A2-9)

The use according to any one of the items, in which a part of the exogenous microorganism contains an enzyme, a nucleic acid encoding an enzyme, a virus, or a metabolite contained in the exogenous microorganism.

### (Item A2-10)

A use of a microorganism having an ability equivalent to that of GI35 strain or GI35 strain or a part thereof for producing a fatty acid other than eicosapentaenoic acid (EPA).

### (Item A2-11)

A use of an unsaturated fatty acid synthase group (for example, SEQ ID NO: 1) derived from GI35 strain or a synthase group having an ability equivalent to that of the unsaturated fatty acid synthase group for producing a fatty acid other than eicosapentaenoic acid (EPA).

### (Item A2-12)

The use according to any one of the items, in which the synthase group contains an extract of the GI35 strain.

### (Item A2-13)

The use according to any one of the items, in which the fatty acid other than EPA is selected from the group consisting of palmitoleic acid, oleic acid, linoleic acid, γ-linolenic acid, α-linolenic acid, stearidonic acid, dihomo-γ-linolenic acid, arachidonic acid, eicosatetraenoic acid (ETA), osbond acid, docosapentaenoic acid (DPA), and docosahexaenoic acid (DHA).

### (Item A3-1)

A use of an exogenous microorganism derived from a gastrointestinal tract of a derived individual different from the target individual, or a part thereof in manufacturing a medicine for improving a target individual of an organism belonging to an organism species having a gastrointestinal tract.

### (Item A3-2)

The use according to any one of the items, in which the improvement is achieved by introducing the exogenous microorganism or a part thereof into a gastrointestinal microflora of the target individual.

### (Item A3-3)

The use according to any one of the items, in which the improvement is achieved by introducing, into the target individual, a feature that is not present in the target individual but is present in the derived individual.

### (Item A3-4)

The use according to any one of the items, in which the improvement is achieved by introducing, into the target individual, the exogenous microorganism or a part thereof that provides a feature that is not present in the target individual but is present in the derived individual.

### (Item A3-5)

The use according to any one of the items, in which the improvement includes at least one selected from the group consisting of modification of nutritional and energy metabolism and modification of immune function and anti-inflammatory and anti-infective function.

### (Item A3-6)

The use according to any one of the items, in which the improvement includes at least one selected from the group consisting of modification of fatty acid metabolism and/or amino acid metabolism, introduction of essential nutrients (for example, essential fatty acids, essential amino acids, vitamins, and the like), growth promotion, infectious disease prevention, enhancement of immune response ability (for example, enhancement of production of a polyunsaturated fatty acid and a lipid mediator that is a metabolite of the polyunsaturated fatty acid in the gastrointestinal tract by an introduced microorganism strain), promotion of utilization of a non-nutrient or a poor nutrient as a nutrient including nutrient conversion of fibers of a photosynthetic organism such as a plant in an animal and introduction of a substance having a high α-amylase activity, modification of diathesis, modification of feeding habit, fiber degradation of a photosynthetic organism including a plant, and metabolism of a polyunsaturated fatty acid.

### (Item A3-7)

The use according to any one of the items, in which the organism is a mammal, a bird, an amphibian, a reptile, fish, a cephalopod, an arthropod, a crustacean, a shellfish, or a wheel animal.

### (Item A3-8)

The use according to any one of the items, in which the organism is an organism provided for food, clothing, fuel, pet, pharmaceutical manufacture, and/or ornamental use.

### (Item A3-9)

The use according to any one of the items, in which a part of the exogenous microorganism contains an enzyme, a nucleic acid encoding an enzyme, a virus, or a metabolite contained in the exogenous microorganism.

### (Item A3-10)

A use of a microorganism having an ability equivalent to that of GI35 strain or GI35 strain or a part thereof in manufacturing a medicine for producing a fatty acid other than eicosapentaenoic acid (EPA).

### (Item A3-11)

A use of an unsaturated fatty acid synthase group (for example, SEQ ID NO: 1) derived from GI35 strain or a synthase group having an ability equivalent to that of the unsaturated fatty acid synthase group in manufacturing a medicine for producing a fatty acid other than eicosapentaenoic acid (EPA).

### (Item A3-12)

The use according to any one of the items, in which the synthase group contains an extract of the GI35 strain.

### (Item A3-13)

The use according to any one of the items, in which the fatty acid other than EPA is selected from the group consisting of palmitoleic acid, oleic acid, linoleic acid, γ-linolenic acid, α-linolenic acid, stearidonic acid, dihomo-γ-linolenic acid, arachidonic acid, eicosatetraenoic acid (ETA), osbond acid, docosapentaenoic acid (DPA), and docosahexaenoic acid (DHA).

In the present disclosure, it is intended that one or the plurality of features can be provided in further combinations in addition to the specified combination. Further embodiments and advantages of the present disclosure will be appreciated by those skilled in the art upon reading and understanding the following detailed description, if necessary.

### Advantageous Effects of Invention

The present disclosure provides a technique capable of improving a target individual of an organism belonging to an organism species having a gastrointestinal tract on a desired basis. For example, it is possible to modify fatty acid metabolism and amino acid metabolism, and it is also possible to achieve introduction of essential nutrients (essential fatty acids, essential amino acids, vitamins, and the like), growth promotion, infectious disease prevention, enhancement of immune response ability by enhancement of production of a polyunsaturated fatty acid and a lipid mediator that is a metabolite of the polyunsaturated fatty acid in the intestinal tract by an introduced strain, and the like. The present disclosure also provides a strain capable of producing a fatty acid other than EPA. The present disclosure can provide an animal target individual that is modified so that a component of a photosynthetic organism that is not a nutrient source in an animal is used as a nutrient source in the animal. It is possible to produce a circulation type “product (meat or the like)” that realizes SDGs and is environmentally friendly. For example, the present disclosure is expected to maintain and promote health and prevent a cardiovascular disease, a lifestyle disease, or the like. In addition, feed containing the present bacterium is administered, such that growth of an animal may be promoted and/or an intestinal bacterial flora may be modified.

### BRIEF DESCRIPTION OF DRAWINGS

FIG. 1 illustrates the results of examining EPA production by Schewanella GI35 strain at a low temperature (4° C.) and a high temperature (18° C.) by gas chromatography (upper chart and lower chart, respectively).

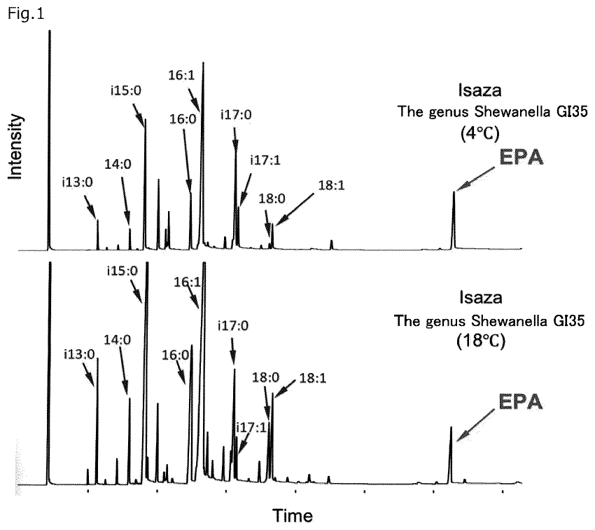

FIG. 2 is a chart showing a procedure of metagenomic analysis.

FIG. 3 illustrates the results of α-diversity analysis of an intestinal bacterial flora of rainbow trout fry fed with GI35 strain.

FIG. 4 illustrates the results of β-diversity analysis of an intestinal bacterial flora of rainbow trout fry fed with GI35 strain.

FIG. 5 is a diagram showing a method for obtaining a variant strain of GI35 having high salt concentration resistance.

FIG. 6 is a diagram showing body weights of red sea bream fry measured after feeding red sea bream fry about 1 month after hatching with normal feed, to which each of the isolated strains (isolated strain-added feed) shown in Table 6 is added, in an amount corresponding to about 4% of the body weight for 10 days, and then breeding the red sea bream fry with the normal feed for 3 months.

### DESCRIPTION OF EMBODIMENTS

Hereinafter, the present disclosure is described while showing some of the best modes thereof. Throughout the present specification, the expression of singular forms is to be understood as including the concept of plural forms unless otherwise stated. Therefore, the singular article (for example, “a”, “an”, “the”, or the like in English) should be understood to include the concept of plural forms unless otherwise stated. In addition, the terms used in the present specification are to be understood as being used in the sense commonly used in the art unless otherwise stated. Therefore, unless defined otherwise, all technical and scientific terms used in the present specification have the same meaning as commonly understood by those skilled in the art to which the present disclosure pertains. In the case of conflict, the present specification (including definitions) will control.

### Definitions

The terms in the present specification are described below.

In the present specification, “about” means±10% of a subsequent numerical value.

In the present specification, the “microflora” means a collection of microorganisms, and is not limited to a gastrointestinal microflora such as an intestinal bacterial flora, and includes, for example, a microflora on the skin or in the mouth, a preparation obtained by mixing a plurality of artificially produced microorganisms, and the like. Therefore, the microflora means a collection of microorganisms such as various bacteria and fungi coexisting in the intestines, skin, mouth, and the like of a human or an animal, and an artificially produced microorganism from these microorganisms. In the present specification, the “improvement of the microflora” includes increasing the variety of microorganisms such as bacteria and fungi constituting the microflora and improving the balance of these microorganisms. In one preferred embodiment, the microflora may refer to a gastrointestinal microflora such as an intestinal bacterial flora, and the present disclosure is not limited thereto. It is understood that the microorganisms are used in the broadest sense used in the art and may also include viruses. In the present specification, among the microflora, when a microorganism does not contain a virus, the microorganism may be referred to as a “non-viral microflora”, when a microorganism is identified as a fungus, a bacterium, or the like, the microorganism may be referred to as a “bacterial flora”, and when a microorganism is a bacterium, the microorganism may be referred to as a “microflora”.

In the present specification, the “organism having a gastrointestinal tract” refers to any organism having a gastrointestinal tract, and the gastrointestinal tract may include the stomach, the intestines, or any gastrointestinal tract corresponding thereto. Examples of the organism having a gastrointestinal tract include, but are not limited to, a mammal, a bird, an amphibian (a frog), a reptile (a turtle or a softshell turtle), fish, a cephalopod (a squid, an octopus, or the like), an arthropod (an insect or the like), a crustacean (a crab, a shrimp, or the like), a shellfish (a bivalve), and a wheel animal (a rotifer or the like). A mammal or animal may be a non-human. The “organism species having a gastrointestinal tract” refers to any organism species belonging to an organism having a gastrointestinal tract. The organism targeted in the present disclosure may be provided for, but is not limited to, food, clothing, fuel, pet, pharmaceutical manufacture, and/or ornamental use. The gastrointestinal tract may be an intestinal tract, and may be expressed as an “organism having an intestinal tract” in this case.

In the present specification, the “individual” refers to an entity that exists individually in each organism species. In the present specification, the “individual” may be referred to as a “target individual” when it is a target of improvement, and may be referred to as a “derived individual” when it is an individual from which an exogenous microorganism or a part thereof is obtained. The “target individual” and the “derived individual” may belong to the same organism species or may belong to different organism species. In a preferred case, it is preferable that the “target individual” and the “derived individual” are the same or similar in the environment in which the gastrointestinal microflora (for example, intestinal bacterial flora) survives, or differ from each other only within a viable level. The individual may be interchangeable with a “target”, an “object to be targeted”, or a “subject”, and may refer to an entity that exists individually of each of a mammal, a bird, an amphibian (a frog), a reptile (a turtle or a softshell turtle), fish, a cephalopod (a squid, an octopus, or the like), an arthropod (an insect or the like), a crustacean (a crab, a shrimp, or the like), a shellfish (a bivalve), and a wheel animal (a rotifer or the like). The mammal or the animal may be fish, poultry such as a chicken, a quail, a turkey, or a duck, a livestock such as a cow, a pig, a goat, sheep, a horse, or a donkey, a pet such as a dog, a cat, a rabbit, or a hamster, and the like as examples in the present specification that may be non-humans. It is considered that by administering the feed of the present disclosure to an organism such as an animal, GI35 strain or a variant strain thereof remains and is continuously present in the intestinal tract of the organism, and EPA is continuously produced in the organism, such that it is useful for promoting health of the organism. Preferably, the feed of the present disclosure is administered to fish. Fish is a collective designation of the classes of Myxini, Cephalaspidomorphi, Chondrichthyes, and Osteichthyes. In the present specification, fish and a fish are synonymous. The feed of the present disclosure can be administered to any type of fish. The feed of the present disclosure can be administered regardless of whether it is a freshwater fish, a seawater fish, or a diadromous fish. The seawater fish or the diadromous fish (salmon, trout, or the like) lacks any of the enzymes required for EPA biosynthesis, or has weak activity of these enzymes, and cannot produce EPA on their own, and therefore, it is effective to administer the feed of the present disclosure to the seawater fish or the diadromous fish. The feed of the present disclosure can be administered regardless of whether it is fry, an immature fish, or an adult fish. Preferably, the feed of the present disclosure is administered to fry or an immature fish. Typically, the feed of the present disclosure is administered to a cultured fish. Examples of the cultured fish include, but are not limited to, salmon, trout, yellowtail, red sea bream, greater amberjack, bluefin tuna, fugu, bastard halibut, white trevally, horse mackerel, yellowtail amberjack, striped beakfish, thread-sail filefish, sea bass, Korean rockfish, carp, rainbow trout, masou salmon, eel, and sweetfish.

In the present specification, the “exogenous microorganism” refers to any microorganism that exists outside the body (including microorganisms present in the gastrointestinal tract such as the intestine), not in the body, for an individual. The “microorganism” is broadly defined, and includes, but is not limited to, a yeast, a mold, a mushroom, a bacterium, an actinomycete, a unicellular alga, a virus, and a protist. In the present disclosure, at least a bacterial group can be suitably used as the microorganism. In the case of an exogenous microorganism, the entity in which it is present may be a host.

In the present specification, the “part of the microorganism” refers to not the whole of a certain microorganism but a part thereof. For example, in the case of a single cell, it may be a part of a cell, for example, an intracellular organelle, a protein, a molecule such as a nucleic acid or a complex thereof, an enzyme contained in an exogenous microorganism, a nucleic acid encoding an enzyme, a virus, a metabolite, or the like.

In the present specification, the “microorganism community” refers to a microflora constituted by a plurality of microorganisms. Note that the microorganism may be evaluated in units of species, or may be evaluated in higher classification hierarchies such as genera and families, or in units of lower levels such as subspecies, varieties, cultivars, lines, an operational taxonomic unit (OTU), or an amplicon sequence variant (ASV). Here, the microorganism may be a germ (including a fungus and a bacterium) or a bacterium as described in the present specification. In the present specification, for convenience, a case using a species as a unit can be described. The “habitat” is a general term for spaces in which microorganism species inhabit, and usually means various liquids, solids, and gases including microorganisms.

In the present specification, the “information of a microorganism species” includes information related to a phylogenetic classification, a genome sequence, a functional gene profile, a gene expression pattern, ecology, a relationship with a host or environment, a state of a host, a state of a habitat of a microorganism, a relationship between a specific microorganism species and another biological species, and the like.

In the present specification, the “proliferation” of a microorganism includes an increase in the absolute number of microorganisms. A degree of increase in the number of microorganisms is not particularly limited, but means that the number of microorganisms is preferably 1.1 times or more, more preferably 1.5 times or more, and still more preferably 3 times or more as compared with a comparative target. Such an increase in the number of microorganisms can be confirmed, for example, by directly measuring the number of microorganisms, measuring a turbidity (absorbance) of contents of a gastrointestinal tract of an animal such as a human that has ingested a medium or a composition in which microorganisms are cultured or the amount of short-chain fatty acids such as acetic acid to increase the value thereof, measuring a pH in the medium to decrease the value thereof, or the like.

In addition, in the present specification, the “proliferation” includes an increase in an abundance ratio of a target microorganism in a gastrointestinal microflora, and the “promotion of proliferation” means that when the composition of the present disclosure is applied, a degree of increase is larger than that when the composition of the present disclosure is not applied. That is, the method includes increasing an abundance ratio of a specific microorganism present in the gastrointestinal tract of the organism of the present disclosure. Here, the “abundance ratio” can also be referred to as an “occupancy ratio” with respect to the entire microorganism community detected in the gastrointestinal microflora. The “increase in abundance ratio” means that an increase or decrease in abundance ratio of other microorganisms in the gastrointestinal microflora may occur simultaneously as long as the abundance ratio of the specific microorganism in the gastrointestinal microflora increases. A degree of increase in the abundance ratio is not particularly limited, and is preferably 2% or more, more preferably 5% or more, and still more preferably 20% or more larger than the abundance ratio of the microorganism in the comparative target. A specific microorganism strain may increase or decrease in the technique of the present disclosure, and this criterion may be used.

In the present specification, the “improvement” refers to introducing, into an organism individual, a biological feature other than the original biological feature of the organism individual, or improving the original biological feature of the organism individual. Here, the biological feature includes arbitrary properties, and can include modification of nutritional and energy metabolism, modification of immune function and anti-inflammatory and anti-infective function, and the like.

In the present specification, the “modification of nutritional and energy metabolism” refers to modification of nutritional features or energy metabolism features for a certain organism. Examples thereof include modification of fatty acid metabolism and amino acid metabolism, introduction of essential nutrients (essential fatty acids, essential amino acids, vitamins, and the like), growth promotion, and promotion of utilization of a non-nutrient or a poor nutrient as a nutrient (for example, nutrient conversion of fibers (for example, plant fibers) of a photosynthetic organism in an animal and introduction of a substance having a high α-amylase activity).

In the present specification, the “modification of immune function and anti-inflammatory and anti-infective function” is also referred to as improvement of homeostasis maintenance function, and refers to modification of an immune function, an action on inflammation, and/or an action or function on infection of a certain organism, and can include, in addition to the infectious disease prevention function, enhancement of immune response ability enhancement of production of a polyunsaturated fatty acid and a lipid mediator that is a metabolite of the polyunsaturated fatty acid in the gastrointestinal tract (for example, the intestine) by an introduced microorganism strain, and the like, improvement of anti-inflammatory action, and the like. Without wishing to be bound by theory for infectious disease control, for example, it is assumed that GI35 bacteria of the present disclosure produce a variety of polyunsaturated fatty acids containing EPA (not reported in previous strains), various lipid mediators are produced in the rainbow trout intestinal tract as demonstrated in the present disclosure, and these lipid mediators confer resistance to infectious diseases because they are involved in the control of inflammation and the enhancement of immune response to infectious diseases in mammals. As for the enhancement of resistance to further infectious diseases, the present inventors have found a finding of the enhancement of resistance to fish disease virus infection (infectious hematopoietic necrosis virus).

In the present specification, the “introduction” of an exogenous microorganism or a part thereof into a target individual or the like refers to any operation that can allow the exogenous microorganism or a part thereof to be engrafted in the target individual to a certain extent (may be in the gastrointestinal tract), and for example, the exogenous microorganism or a part thereof can be ingested by being mixed with feed, or in the case of a rotifer, the exogenous microorganism or a part thereof can be taken by being added to a breeding water tank. In a case of an animal such as a mouse, an experiment can proceed by oral administration by force, and the exogenous microorganism or a part thereof can also be additionally ingested during drinking water.

In the present specification, the “part of time” means that the time of introduction includes at least a part of the whole when introducing a microorganism, an enzyme, a part thereof, or the like. The part of time may be a single diet as long as a microorganism, an enzyme, or a part thereof is substantially effective after introduction. In a case where introduction is made during a part of time, introduction may not be made during the rest of the time, and similar or different introduction may be made. The part of time of introduction may be during growth (in this case, fish are fry), after growth (in the case of fish, adult fish), or both.

In the present specification, the “disease”, “disorder”, and “condition” are used interchangeably and interpreted in the broadest sense, refer to a state in which weak or inconvenience has occurred in the heart or body of a human being or an animal, and refer to any state that cannot be said to be a health state that is not specifically defined, such as a disease, a disorder, and various symptoms.

In the present specification, the “infectious disease” may be any infectious disease, including any type of infectious disease such as a viral infectious disease (including any viral form such as a single-stranded or double-stranded DNA virus or RNA virus), a bacterial infectious disease, a protozoal infectious disease, or a mycoplasma infectious disease, and may be, for example, tuberculosis, coronavirus, malaria, yellow fever virus, smallpox virus, vaccinia, measles/rubella, polio, epidemic parotitis/MUMPS, rotavirus infection, chicken pox, yellow fever, Ebola, West Nile Fever, Hib infection, pneumococcus infection, whooping cough, Japanese encephalitis, meningococcal infection, salmonella infection, pathogenic E. coli, toxoplasma, zika virus, herpes virus type 1, EBV/Epstein-Barr virus (herpes virus type 4), CMV/cytomegalovirus (herpes virus type 5), influenza, MARS, rabies, and diphtheria.

In the present specification, the “prevention” is an action of administering the active ingredient of the present disclosure to an individual who has not developed a disease to be targeted, and for example, an object thereof is to prevent the onset of a disease. A vaccine can be said to be a representative example of a medicine for the purpose of prevention. In the present disclosure, even in a case where a causative factor of a disease is present in a target, it is not usually determined as a disease state unless the disease is developed. Therefore, even such a state can be a target of treatment, and it can be said that prevention is performed.

In the present specification, the “treatment” is, for example, an act of administering the active ingredient of the present disclosure to an individual (a subject or a patient) diagnosed as having developed a disease by a doctor or an equivalent practitioner thereof, and has the purpose of, for example, reducing the disease or symptoms, reducing the number of causative viruses or organisms of infectious diseases in a subject, or returning to a state before the onset of the disease. In addition, even when the purpose of administration is to prevent diseases or symptoms from worsening or to reduce the number of viruses or organisms that cause infectious diseases, as long as it is administered to a patient, it corresponds to treatment.

In the present specification, the “GI35 strain” refers to the Shewanella sp. GI35 strain, which is a strain that has been deposited with the National Institute of Technology and Evaluation, Patent Microorganisms Depositary, having address #122, 2-5-8 Kazusakamatari, Kisarazu-shi, Chiba 292-0818, under the Budapest Treaty on the International Recognition of the Deposit of Microorganisms for the Purposes of Patent Procedure, and given an accession number NITE BP-03244 as of Aug. 25, 2020 (depositary administrator: HOLO BIO Co., Ltd.).

The GI35 strain mass-produces eicosapentaenoic acid (EPA) (comparison of several times the amount of Shewanella livingstonesis Ac10 strain, which is highly producing EPA, with literature values), survives well in the intestinal tract, and continuously exists. Moreover, as a bacterium belonging to the genus Shewanella, the GI35 strain proliferates well even at a relatively high temperature (room temperature, for example, about 18° C.), and produces a large amount of EPA. The GI35 strain is a novel strain characterized by these special properties.

Thus, in the present specification, the GI35 strain includes a variant strain thereof, in particular, a microorganism having a comparable ability. In the present specification, the “microorganism having an ability equivalent to that of the GI35 strain” refers to having at least one of the features of the GI35 strain, and in particular, may include an unsaturated fatty acid synthase group (including, but not limited to, SEQ ID NO: 1) derived from a fatty acid metabolism, particularly the GI35 strain, or a synthase group having an ability equivalent to that of the unsaturated fatty acid synthase group.

In the present specification, the “synthase group having an ability equivalent to that of” a certain unsaturated fatty acid synthase group refers to a synthase group having an ability equivalent to that of a target enzyme group. Such a synthase group can also be achieved by using a fraction having a specific synthetic ability in a fraction obtained from a microorganism having target activity although individual genes have not been isolated, other than those in which individual genes have been identified.

The microorganism having an ability equivalent to that of the GI35 strain may be a variant strain derived from the GI35 strain. The microorganism having an ability equivalent to that of the GI35 strain may be a natural variant strain or an artificial variant strain. A method for producing an artificial variant strain is known, and examples thereof include, but are not limited to, genetic recombination, genome editing, treatment with a drug such as N-methyl-N′-nitro-N-nitrosoguanidine (NTG) or ethyl methanesulfonic acid (EMS), and ultraviolet radiation. Examples of the variant strain of the GI35 strain include, but are not limited to, a strain having a higher ability to produce EPA than the GI35 strain, a strain that proliferates well at a higher temperature, and a strain having excellent intestinal colonization properties. The whole genome sequence of the variant strain of the GI35 strain may have a homology of 70% or more, preferably 80% or more, more preferably 90% or more, still more preferably 95% or more, and most preferably 98% or more, with respect to the whole genome sequence of the GI35 strain. A sequence homology between genomes can be examined using known programs such as FASTA and BLAST. However, the variant of the GI35 strain has an EPA production ability equivalent to that of the GI35 strain. Here, an equivalent EPA production ability means 70% or more, preferably 80% or more, more preferably 90% or more, still more preferably 100% or more, and most preferably 120% or more.

The present disclosure, in a further aspect, can be provided as a composition (for example, feed) containing GI35 strain or a microorganism having an ability equivalent to that of the GI35 strain. The organism to which the feed of the present disclosure is administered may be any kind of organism, and is not particularly limited.

In the present specification, the “unsaturated fatty acid” is used in a broad definition used in the art, and a polyvalent unsaturated fatty acid is referred to as a “polyunsaturated fatty acid”. Examples of such an unsaturated fatty acid include palmitoleic acid, oleic acid, linoleic acid, γ-linolenic acid, α-linolenic acid, stearidonic acid, dihomo-γ-linolenic acid, arachidonic acid, eicosatetraenoic acid (ETA), osbond acid, EPA, docosapentaenoic acid (DPA), and docosahexaenoic acid (DHA). A fatty acid synthesis system of the GI35 strain of the present disclosure is significantly unique and can biosynthesize a variety of highly polyunsaturated acids such as EPA (C20:5, n3), as well as DHA (C22:6, n3), arachidonic acid (C20:4, n6), and stearidonic acid (C18:4, n3).

In the present specification, the “individual exhibiting the improvement” means that a certain individual has desired “improvement” (for example, nutritional properties or the like) in the present disclosure.

In the present specification, the “suitable” refers to being capable of directly or indirectly supporting the intended improvement (for example, requirements such as metabolism and nutrition), and is also referred to as being compatible or the like. What is suitable varies depending on the intended improvement, and those skilled in the art can appropriately select according to the intended improvement in light of the description in the present specification and taking into consideration common technical knowledge.

In the present specification, the “nutrient source” refers to any substance that provides any substance necessary for survival to an organism. For an organism, for example, fibers are a nutrient source, but in many animals, including a human, fibers are not a nutrient source. The nutrition may be any nutrition, and is not limited to those selected from the group consisting of a lipid, a carbon source (carbohydrate), cellulose, hemicellulose, and lignin as woody biomass, an amino acid, a vitamin, a carotenoid, and a mineral.

In the present specification, a substance being “nutritionally available” means that an organism can utilize a substance to be targeted as a nutrient, and for example, fibers are nutritionally available for an organism having a microorganism capable of degrading fibers.

In the present specification, the “microflora”, for example, the intestinal microflora can be a factor that determines an organism's individuality. Such individuality be expressed by a diversity index (Shannon index or the like) of the metagenomic analysis result, and can be expressed by a value different from a value found in nature (diversity is increased or decreased).

In the present specification, the diversity index may be expressed by an α-diversity index and a β-diversity index. The α-diversity index represents a diversity of one sample. That is, the index is an index unique to a sample, the larger the value, the higher the diversity of species, and depending on the index, the difference is whether to emphasize “the number of observed species” or whether “the respective species are observed equally”. The β-diversity index represents the degree of difference in diversity between two samples, and is an index expressed as a distance between two points. The greater the distance, the more different the composition of the two samples.

In the present specification, the “product” may be any substance produced by an individual to be targeted or a complex composed of the substance, and examples thereof include food such as meat or milk, clothing such as leather, pharmaceuticals, and raw materials thereof.

In the present specification, the “processed product” is not any substance produced by an individual to be targeted or a complex composed of any substance itself, but is any substance obtained by processing the substance or a complex thereof, and examples thereof include a meat processed product (hamburg steak or the like), a dairy product (cheese or the like), and a canned product.

In the present specification, the “kit” refers to a unit providing portions to be provided (for example, a composition of the present disclosure, additional components, a buffer, a manual, and the like) that are usually divided into two or more sections. The form of the kit is preferred when it is intended to provide a composition that should not be provided in a mixed state for stability or the like, but is preferably mixed immediately before use or administered separately. Such a kit preferably includes an instruction or manual describing how to use or process the provided portions (for example, a composition and additional components). When the kit is used in the present specification, the kit generally includes an instruction or the like describing how to use the components or composition of the present disclosure and the like.

In the present specification, the “instruction” is a document with an explanation of the method of use of the present disclosure for a user. The instruction includes language directing how to use the present disclosure. The instruction is prepared in accordance with a format defined by the regulatory agency of the country in which the present disclosure is practiced (for example, the Ministry of Health, Labor and Welfare in Japan or the like, and Food and Drug Administration (FDA) in the United States), with an explicit description showing approval by the regulatory agency, if necessary. The instruction may be usually provided in, but is not limited to, a paper medium. For example, the instruction may also be provided in a form such as an electronic medium (for example, a web site or an e-mail provided on the Internet).

### DESCRIPTION OF PREFERRED EMBODIMENTS

Preferred embodiments of the present disclosure will be described below. It is understood that the embodiments provided below are provided for a better understanding of the present disclosure, and that the scope of the present disclosure should not be limited to the following description. Therefore, it is apparent that those skilled in the art can appropriately make modifications within the scope of the present disclosure in view of the description in the present specification. It is also understood that the following embodiments of the present disclosure may be used alone or in combination.

### (Novel Breeding Technique)

The present disclosure relates to a technique such as a method, use, composition, or drug for improving a target individual of an organism belonging to an organism species having a gastrointestinal tract by introducing an exogenous microorganism derived from a gastrointestinal tract of a derived individual different from the target individual or a part thereof into the target individual of the organism belonging to the organism species having the gastrointestinal tract.

In one aspect, the present disclosure provides a composition for use in improving a target individual of an organism belonging to an organism species having a gastrointestinal tract, the composition containing an exogenous microorganism derived from a gastrointestinal tract of a derived individual different from the target individual, or a part thereof.

In one embodiment, the improvement is achieved by introducing the exogenous microorganism or a part thereof into an intestinal bacterial flora of the target individual. The introduction may be any type as long as a target exogenous microorganism or a part thereof is introduced, but the exogenous microorganism or a part thereof can be mixed with feed and ingested, or can be taken by being added to a breeding water tank. In a case of a large animal, an experiment can proceed by oral administration by force, and the exogenous microorganism or a part thereof can also be added during drinking water. Furthermore, other than the above, the intestinal bacterial flora can be introduced via the feces of the derived individual, and the feces may be directly obtained from the derived individual, or may be adjusted by adding another microorganism or the like. In addition, it can also be introduced into a target individual by being introduced in advance into a photosynthetic organism such as an animal or a plant used as feed.

In one embodiment, the present disclosure includes not only direct modification but also indirect modification. For example, in one embodiment, the improvement includes introducing, into the target individual, a feature that is not present in the target individual but is present in the derived individual.

In one embodiment, in the modification, the exogenous microorganism or a part thereof may be fed only at an early stage of breeding, and then breeding may be performed using a feed that does not contain the exogenous microorganism or a part thereof. This leads to a reduction in burden on the environment and a reduction in breeding costs caused by addition of the exogenous microorganism or a part thereof. For example, the exogenous microorganism or a part thereof may be fed only for 1 day, 2 days, 3 days, 4 days, 5 days, 6 days, 7 days, 8 days, 9 days, 10 days, 2 weeks, 3 weeks, or 4 weeks after the target individual begins to eat the feed, and then breeding may be performed using a feed that does not contain the exogenous microorganism or a part thereof.

In one embodiment, the improvement includes introducing, into the target individual, the exogenous microorganism or a part thereof that provides a feature that is not present in the target individual but is present in the derived individual. In another embodiment, the improvement can be achieved by a change resulting from introducing the exogenous microorganism or a part thereof into the target individual (for example, a change in the intestinal bacterial flora reduces harmful bacteria). In such a case, although the exogenous microorganism or a part thereof that provides a feature that is not present in the target individual but is present in the derived individual is not necessarily introduced, a desired improvement can be achieved and is also part of the present disclosure.

In one embodiment, the improvement includes, but is not limited to, modification of nutritional and energy metabolism, modification of immune function and anti-inflammatory and anti-infective function, and the like.

In a preferred embodiment, the improvement may be modification of fatty acid metabolism and amino acid metabolism, introduction of essential nutrients (essential fatty acids, essential amino acids, and vitamins), growth promotion, infectious disease prevention, enhancement of immune response ability (for example, enhancement of production of a polyunsaturated fatty acid and a lipid mediator that is a metabolite of the polyunsaturated fatty acid in the gastrointestinal tract by an introduced microorganism strain), promotion of utilization of a non-nutrient or a poor nutrient as a nutrient including nutrient conversion of fibers of a photosynthetic organism such as a plant in an animal and introduction of a substance having a high α-amylase activity, modification of diathesis, modification of feeding habit, fiber degradation of a photosynthetic organism including a plant, metabolism of a polyunsaturated fatty acid, and the like.

In a certain embodiment, the organism to be targeted may be a mammal, a bird, an amphibian (a frog), a reptile (a turtle or a softshell turtle), a fish, a cephalopod (a squid, an octopus, or the like), an arthropod (an insect or the like), a crustacean (a crab, a shrimp, or the like), a shellfish (a bivalve), and a wheel animal (a rotifer or the like). These organisms may be organisms provided for food, clothing, fuel, pet, pharmaceutical manufacture, and/or ornamental use (including a case of being used as, for example, wool or fish oil other than food). The organism may be a non-human.

In one embodiment, a part of the exogenous microorganism contains an enzyme, a nucleic acid encoding an enzyme, a virus, or a metabolite contained in the exogenous microorganism. An enzyme, a nucleic acid encoding an enzyme, a virus, or a metabolite contained in the exogenous microorganism may provide a desired improvement, and these improvements can be achieved by introducing all or part of an enzyme, a nucleic acid encoding an enzyme, a virus, or a metabolite contained in the exogenous microorganism.

In another aspect, the present disclosure provides a method for producing an improved target organism, the method including: a step A) of selecting an individual exhibiting the improvement from candidate microorganisms of an organism species to which a derived individual different from the target individual belongs; a step B) of obtaining, in a derived individual exhibiting the improvement, an exogenous microorganism involved in the improvement or a part thereof from a gastrointestinal bacterial flora of the derived individual; a step C) of introducing the exogenous microorganism or a part thereof into the target individual; and a step D) of optionally confirming the properties of the gastrointestinal bacterial flora in the target individual, and confirming that a desired improvement is achieved.

In one embodiment, the step A) of selecting an individual exhibiting the improvement from candidate microorganisms of an organism species to which a derived individual different from the target individual belongs can be performed in any form. For example, in a case where, for example, an ability to degrade fibers is selected as an improvement in a target derived individual, a specific individual can be selected from candidate microorganisms of a microorganism species to which the derived individual belongs by selecting an individual capable of degrading the fibers by an arbitrary method.

In one embodiment, the step B) of obtaining, in a derived individual exhibiting the improvement, an exogenous microorganism involved in the improvement or a part thereof from a gastrointestinal bacterial flora of the derived individual can be performed in any form, and the exogenous microorganism or a part thereof can be obtained from the gastrointestinal bacterial flora of the individual exhibiting the improvement by any method, and can be performed by various screening methods.

In one embodiment, the step C) of introducing the exogenous microorganism or a part thereof into the target individual can be achieved by disposing the selected exogenous microorganism or a part thereof in the target individual by any method, and for example, the selected exogenous microorganism or a part thereof can be mixed into feed and ingested or can be taken by being added to a breeding water tank. In a case of a large animal, an experiment can proceed by oral administration by force, and the exogenous microorganism or a part thereof can also be added during drinking water.

In one embodiment, the step C) may include a step of introducing the exogenous microorganism or a part thereof into the target individual at least at a part of a time during breeding of the target individual. In one embodiment, the step C) may include a step of introducing the exogenous microorganism or a part thereof into the target individual at least at a part of a time during breeding of the target individual, and breeding the target individual without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks.

In one embodiment, the step C) may include a step of introducing the exogenous microorganism or a part thereof into the target individual at least at a part of a time during growing of the target individual. In one embodiment, the step C) may include a step of introducing the exogenous microorganism or a part thereof into the target individual at least at a part of a time during growing of the target individual, and breeding the target individual without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks. In some embodiments, the growth time can be any period of time during which the body length increases, and in some embodiments, the growth time can be any period of time after beginning of postpartum feed or water ingestion, and can be 1 day, 2 days, 3 days, 4 days, 5 days, 6 days, 7 days, 8 days, 9 days, 10 days, 2 weeks, 3 weeks, or 4 weeks after beginning of postpartum feed or water ingestion. The exogenous microorganism or a part thereof is introduced into the target individual during the time, such that the exogenous microorganism or a part thereof can be stably maintained in the bacterial flora in the body of the target individual.

In one embodiment, the method of the present invention may further include a step of optionally confirming that a desired improvement is achieved in the target individual. In a certain embodiment, the desired improvement may be a change (increase or decrease) in body weight, a change in intestinal bacterial flora, a change (increase or decrease) in fat composition in the body, a change (increase or decrease) in fat percentage in body weight, an increase in intake, or an improvement in digestion and absorption of plant feed.

In one embodiment, the method of the present invention may further include a step B′) of selecting an exogenous microorganism suitable (“compatible”) for the target individual, or a part thereof, and a step C′) of introducing the suitable exogenous microorganism or a part thereof into the target individual. In a certain embodiment, the exogenous microorganism or a part thereof suitable for the target individual may be an exogenous microorganism or a part thereof that results in a desired improvement or is susceptible to engraftment in the target individual. In some embodiments, when the target individual is an individual living in seawater, the exogenous microorganism or a part thereof suitable for the target individual may be an exogenous microorganism derived from an organism living in seawater or a part thereof. In some embodiments, when the target individual is an individual living in freshwater, the exogenous microorganism or a part thereof suitable for the target individual may be an exogenous microorganism derived from an organism living in freshwater or a part thereof. In some embodiments, when the target individual is an individual living in brackish water, the exogenous microorganism or a part thereof suitable for the target individual may be an exogenous microorganism derived from an organism living in brackish water or a part thereof.

The culture medium used for the exogenous microorganism may be either a natural medium or a synthetic medium as long as the culture medium usually contains a carbon source, a nitrogen source, inorganic salts, and the like and can efficiently culture the bacterial species described above. As the carbon source, for example, lactose, glucose, sucrose, fructose, galactose, molasses, and the like can be used, and as the nitrogen source, an organic nitrogen-containing substance such as casein hydrolysate, whey protein hydrolysate, soybean protein hydrolysate, yeast extract, or meat extract can be used. In addition, as the inorganic salts, phosphate, sodium, potassium, magnesium, manganese, iron, zinc, and the like can be used. Examples of the medium suitable for the culture include MRS liquid medium, GAM medium, BL medium, Briggs Liver Broth, animal milk, skimmed milk, and milk whey. Preferably, sterile MRS medium can be used. In addition, in the case of use in food applications, a medium composed only of a food material and a food additive can be adjusted and used. As the natural medium, tomato juice, carrot juice, other vegetable juices, apple, pineapple, grape juice, or the like can also be used.

The culture of the exogenous microorganism is performed under an anaerobic condition at 20° C. to 50° C., preferably at 25° C. to 42° C., more preferably at about 37° C. In the case of the exogenous microorganism derived from fish, the culture may be performed at 18° C. to 30° C. The temperature condition can be adjusted by a thermostatic bath, a mantle heater, a jacket, or the like. In addition, the anaerobic condition is a low oxygen environment in which microorganisms can proliferate, and can be set to an anaerobic condition by using, for example, an anaerobic chamber, an anaerobic box, a sealed container or bag containing a deoxidizing agent, or the like, or by simply sealing a culture container. The type of culture includes static culture, shaking culture, tank culture, and the like. In addition, the culture time is not particularly limited, and can be, for example, 3 hours to 96 hours. The pH of the culture medium at the start of culture is preferably maintained at, for example, 4.0 to 8.0.

As an example, the exogenous microorganism can be inoculated into a food grade medium and cultured overnight (about 18 hours) at about 37° C.

After the culture, the obtained exogenous microorganism culture may be used as it is, or if necessary, crude purification by centrifugation or the like and/or solid-liquid separation by filtration or the like or sterilization operation may be performed. Preferably, centrifugation is performed to recover only cells of the exogenous microorganism. Note that the exogenous microbial bacteria used in the present disclosure may be wet microbial cells or dry microbial cells.

In one embodiment, a step D) of optionally confirming the properties of the gastrointestinal microflora in the target individual, and confirming that a desired improvement is achieved can be performed, and in this step, it can be confirmed whether the improvement is included in the target individual as appropriate according to the type of improvement. For example, when it is an improvement that a fiber can be used as a nutrient source, it is only required to check whether the fiber can be provided as a nutrient.

The gastrointestinal tract in the present disclosure may be any gastrointestinal tract, and may be the entire gastrointestinal tract when the stomach and the intestine are not separated, or may be the stomach and the intestine. In a preferred embodiment, the gastrointestinal tract in the present disclosure is an intestine. In this case, a gastrointestinal microorganism is called an intestinal microorganism. When microorganisms form a flora, the gastrointestinal microflora is referred to as an intestinal bacterial flora.

In a preferred embodiment, the improvement includes at least one selected from the group consisting of modification of nutritional and energy metabolism and modification of immune function and anti-inflammatory and anti-infective function, examples thereof include modification of fatty acid metabolism and amino acid metabolism, introduction of essential nutrients (essential fatty acids, essential amino acids, vitamins, and the like), growth promotion, infectious disease prevention, enhancement of immune response ability by enhancement of production of a polyunsaturated fatty acid and a lipid mediator that is a metabolite of the polyunsaturated fatty acid in the intestinal tract by an introduced strain, promotion of utilization of a non-nutrient or a poor nutrient as a nutrient (for example, nutrient conversion of photosynthetic organism fibers such as plant fibers in an animal and introduction of a substance having a high α-amylase activity), modification of diathesis, modification of feeding habit, fiber degradation of a photosynthetic organism such as plant fibers, and metabolism of a polyunsaturated fatty acid, and the improvement can be achieved through the breeding provided by the present disclosure.

In one embodiment, the exogenous microorganism may be present outside the target organism, and preferably in the gastrointestinal tract, including the intestines.

In one embodiment, the improvement may also be considered to be nutritional, and includes, for example, a method for producing an organism having improved or altered nutrient availability, the method including: a step of introducing, into a gastrointestinal bacterial flora, a microorganism or enzyme that imparts a metabolic activity of using a component that is not a nutrient source for the organism as a nutrient source for the organism and/or improves the metabolic activity of the organism for the component that is a nutrient source for the organism.

In one embodiment, the step of introducing the microorganism or enzyme that improves the metabolic activity of the organism into the gastrointestinal microflora may include a step of introducing the microorganism or enzyme that improves the metabolic activity of the organism into the organism at least at a part of a time during breeding of the organism. In one embodiment, the step of introducing the microorganism or enzyme that improves the metabolic activity of the organism into the gastrointestinal microflora may include a step of introducing the microorganism or enzyme that improves the metabolic activity of the organism into the organism at least at a part of a time during breeding of the organism, and breeding the organism without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks.

In one embodiment, the step of introducing the microorganism or enzyme that improves the metabolic activity of the organism into the gastrointestinal microflora may include a step of introducing the microorganism or enzyme that improves the metabolic activity of the organism into the organism at least at a part of a time during growing of the organism. In one embodiment, the step of introducing the microorganism or enzyme that improves the metabolic activity of the organism into the gastrointestinal microflora may include a step of introducing the microorganism or enzyme that improves the metabolic activity of the organism into the organism at least at a part of a time during growing of the organism, and breeding the organism without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks. In some embodiments, the growth time can be any period of time during which the body length increases, and in some embodiments, the growth time can be any period of time after beginning of postpartum feed or water ingestion, and can be 1 day, 2 days, 3 days, 4 days, 5 days, 6 days, 7 days, 8 days, 9 days, 10 days, 2 weeks, 3 weeks, or 4 weeks after beginning of postpartum feed or water ingestion. The exogenous microorganism or a part thereof is introduced into the organism during the time, such that the exogenous microorganism or a part thereof can be stably maintained in the bacterial flora in the body of the organism.

In one embodiment, the method of the present invention may further include a step of optionally confirming that a desired improvement is achieved in the organism. In a certain embodiment, the desired improvement may be a change (increase or decrease) in body weight, a change in intestinal bacterial flora, a change (increase or decrease) in fat composition in the body, a change (increase or decrease) in fat percentage in body weight, an increase in intake, or an improvement in digestion and absorption of plant feed.

In one embodiment, the method of the present invention may further include a step B′) of selecting a microorganism or enzyme that improves the metabolic activity of the organism suitable (“compatible”) for the organism; and a step C′) of introducing the microorganism or enzyme that improves the metabolic activity of the organism into the organism. In a certain embodiment, the exogenous microorganism or a part thereof suitable for the organism may be an exogenous microorganism or a part thereof that results in a desired improvement or is susceptible to engraftment in the organism. In some embodiments, when the organism is an individual living in seawater, the exogenous microorganism or a part thereof suitable for the organism may be an exogenous microorganism derived from an organism living in seawater or a part thereof. In some embodiments, when the organism is an individual living in freshwater, the exogenous microorganism or a part thereof suitable for the organism may be an exogenous microorganism derived from an organism living in freshwater or a part thereof. In some embodiments, when the organism is an individual living in brackish water, the exogenous microorganism or a part thereof suitable for the organism may be an exogenous microorganism derived from an organism living in brackish water or a part thereof.

In one embodiment, the nutrition may be a lipid (lipid including an essential fatty acid), a carbon source (carbohydrate, fibers (for example, cellulose, hemicellulose, and lignin as woody biomass)), an amino acid, a vitamin, a mineral, a carotenoid, or the like.

In another embodiment, the method of the present disclosure includes allowing a substance that is not nutritionally available in the target organism to be nutritionally available.

In a certain embodiment, a method of using a component of a photosynthetic organism, such as a plant, as a nutrient source is provided, and for example, there is provided a method for producing a target individual of an animal modified to have a component of a photosynthetic organism such as a plant that is not a nutrient source in the animal as a nutrient source in the animal, the method including: a step A) of providing an exogenous microorganism having an ability to convert the component of the photosynthetic organism such as a plant into a nutrient source in the animal, or a part thereof; and a step B) of introducing the exogenous microorganism or a part thereof into the target individual. Here, the step of providing the exogenous microorganism having the ability to convert the component of the photosynthetic organism such as a plant or a part thereof into the nutrient source in the animal can be performed by selecting an exogenous microorganism or a part thereof that contains an enzyme (lignase, hemicellulase, cellulase, xylanase, pectinase, glucanase, laccase, lactase, or the like) that imparts an ability to convert a component (for example, fiber) of a photosynthetic organism such as a plant into a nutrient source in the animal and providing the exogenous microorganism or a part thereof. In the step B) of introducing the exogenous microorganism or a part thereof into the target individual, as described elsewhere in the present specification, the introduction may be any type as long as a target exogenous microorganism or a part thereof is introduced, but the exogenous microorganism or a part thereof can be mixed with feed and ingested, or can be taken by being added to a breeding water tank. In a case of a large animal, an experiment can proceed by oral administration by force, and the exogenous microorganism or a part thereof can also be added during drinking water.

Here, the animal to be targeted in the present disclosure may be a mammal, a bird, an amphibian (a frog), a reptile (a turtle or a softshell turtle), a fish, a cephalopod (a squid, an octopus, or the like), an arthropod (an insect or the like), a crustacean (a crab, a shrimp, or the like), a shellfish (a bivalve), and a wheel animal (a rotifer or the like). These organisms may be organisms provided for food, clothing, fuel, pet, pharmaceutical manufacture, and/or ornamental use (including a case of being used as, for example, wool or fish oil other than food).

The plant to be targeted in the present disclosure may be a tree, a grass, or an alga such as a seaweed.

In the present specification, the “photosynthetic organism” refers to an organism that can utilize light as a direct energy source, and includes a plant and an alga.

In the present specification, the “alga” refers to a generic term of organisms that perform oxygen-generating photosynthesis, excluding plants (bryophytes, ferns, seed plants, etc.) that mainly inhabit on the ground, and includes a blue-green alga, a green alga, a microalga, and the like. The alga includes evolutionarily distinct groups, ranging from cyanobacteria (blue-green algae), which are eubacteria, to eukaryotic unicellular organisms (diatoms, yellow-green algae, dinoflagellates, and the like) and multicellular marine algae (red algae, brown algae, and green algae).

The plant as used in the present specification may be a plant or a part thereof. Alternatively, the plant may be provided not only in a living state but also in a non-living state such as a harvested product or a processed product as long as plant fibers can be obtained.

In the present specification, the “plant” or “plant body” is used in the broadest sense in the art, refers to those that perform a biological phenomenon, and refers to those that live without exercise by performing photosynthesis. Typically, a plant body has various characteristics such as cell structure, proliferation (self-reproduction), growth, regulatory properties, substance metabolism, and repair ability, and usually has, as basic attributes, proliferation involving heredity responsible for nucleic acids and metabolism responsible for proteins. The plant body may be either an angiosperm or gymnosperm cell, may be either a dicotyledonous or monocotyledonous cell, or may be either a herbaceous plant or woody plant. Examples of the herbaceous plant include a grain plant, a turf grass, and a vegetable, and examples of the woody plant include an evergreen broad-leaved tree and a deciduous broad-leaved tree. Specific examples thereof include, but are not limited to, agricultural and horticultural crops such as rice, wheat, barley, corn, grape, apple, pear, peach, cherry, persimmon, citrus, soybean, bean, strawberry, potato, cabbage, lettuce, tomato, cucumber, eggplant, watermelon, sugar beet, spinach, sugar pea, pumpkin, sugar cane, tobacco, green pepper, sweet potato, taro, konjac, cotton, sunflower, tulip, chrysanthemum, and grass. In addition, the “plant body” referred to in the present invention refers to a plant body including all parts constituting the plant individual.

In the present specification, the “part of a plant body” may be, for example, a specific part of a plant body such as a stem, a leaf, a root, a seed, a flower, or a fruit, or may be a combination of a plurality of organs including a stem, a leaf, a seed, and the like. A part of the plant body may include a site such as a ground part (for example, a leave, a stem, and a node, or a leave, a stem, a node, an ear) or an underground part.

In the present specification, the “seed” refers to one that stores nutrients for germination of a young plant and is used for agricultural breeding. Specific examples thereof include grains such as rice, corn, cottonseed, wheat, and barley; gramineous grains such as pearl millet, foxtail millet, proso millet, finger millet, Japanese barnyard millet, barnyard millet, kodo millet, sorghum, adlay, oat, and rye; sunflower seeds; pumpkin seeds; beans; and rapeseeds.

In the present specification, the “crop's edible portion” refers to an edible portion such as seeds of grains and fruits of fruit trees. The crop's edible portion is a concept mainly including seeds and fruits.

In the present specification, the “ground part” refers to a part of a plant body, and refers to a part including a leave and a stem during a vegetative growth stage, and a leave and stem, a flower stem, and a flower during a reproductive growth stage. For example, the “ground part” during the vegetative growth stage in the gramineous plant is a part consisting of a leave, a stem, and a node, and the “ground part” during the reproductive growth stage is a part consisting of a leave, a stem, a node, and an ear (a branch and a floret).

In the present disclosure, the plant body may be a part of a plant body provided for food other than seeds. For example, a part of the plant body may be a fruit of a vegetable such as tomato, cucumber, eggplant, snow pea, pumpkin, or bell pepper. Alternatively, a part of the plant body may be a leaf vegetable such as spinach, mizuna, and nozawana. A part of the plant body may also be used to eat an underground part such as taro, potato, sweet potato, konjac, lotus root, or lily root. The plant and a part thereof utilized in the present disclosure may also not be edible. The plant body or a part thereof may be a bulb such as a seed tuber, lily, and tulip, a seed ball such as Japanese scallion, or the like.

Examples of the turf grass include a gramineous turf grass [for example, an eragrostis subfamily (for example, Zoysia or Bermuda grass), a festuca subfamily (for example, bent grass, blue grass, fescue, or ryegrass), or a millet subfamily], a cyperaceae grass, or a chrysanthemum grass. Examples of the grain plant include a gramineous plant, for example, rice, rye, barley, wheat, millet, sorghum, sugar cane, corn/popcorn, or oat. Examples of the vegetable include a solanaceae plant (for example, tobacco, eggplant, potato, tomato, or hot pepper), a chenopodiaceous plant (for example, spinach, or sugar beet), a papilionaceous plant (for example, soybean, azuki bean, or pea), a brassicaceous plant (for example, oilseed rape or arugula), or a pedaliaceaeous plant (for example, sesame).

Examples of the evergreen broad-leaved tree include eucalyptus, acacia, and coffee. Examples of the deciduous broad-leaved tree include poplar, sawtooth oak, willow, white birch, and Quercus serrata.

In addition, a plant generally known as a house plant (for example, a plant such as Agavaceae, Araceae, Arecaceae, Araliaceae, Moraceae, Asclepiadaceae, Acanthaceae, Apocynaceae, Marantaceae, Cupressaceae, Rutaceae, Bombacaceae, Pandanaceae, Musaceae, Euphorbiaceae, Oleaceae, Commelinaceae, Bromeliaceae, Crassulaceae, Asparagaceae, or Salicaceae, a fern, or the like) is also assumed.

In one embodiment, the photosynthetic organism includes any kind of a plant and an alga, and examples of the plant include, but are not limited to, a gramineous plant such as rice, wheat, corn, sugar cane, silver grass, and reed, a conifer such as Japanese cedar, Japanese cypress, ginkgo, and pine, and a broad-leaved tree such as beech, Japanese oak, sawtooth oak, oak, and Japanese elm. In addition, rice bran or wood chips and corrugated cardboard obtained by processing a plant, and the like are also included, an alga is also included, and examples of the alga include seaweed such as wakame, turnip, konbu, nori, or Japanese agar, and other microalgae.

In one embodiment, the nutrition (component of the photosynthetic organism such as a plant) may be an essential fatty acid, a carbon source, carbohydrate, cellulose, hemicellulose, and lignin as woody biomass, an amino acid, the like.

In one embodiment, the exogenous microorganism used may be an exogenous microorganism capable of converting the nutrient source in a normal growth environment of the animal.

In a specific embodiment, the intestinal microorganism is an intestinal bacterium of Japanese rice fish, and the nutrition may include at least one of a photosynthetic organism fiber such as a plant, for example, cellulose, hemicellulose, and lignin. Examples of the intestinal bacterium of Japanese rice fish may be described elsewhere in the present specification. Without wishing to be bound by theory, since Japanese rice fish can grow from a high temperature (37° C. or higher) to a low temperature (such as 4° C.), the intestinal bacteria are also understood to have these broad temperature band activities.

In one embodiment, as the intestinal bacteria, the present disclosure provides a novel microorganism derived from Japanese rice fish having an ability to degrade at least one selected from the group consisting of cellulose, hemicellulose, and lignin. The microorganism may be, for example, a species of the genus Pseudomonas, the genus Microbacterium, the genus Aeromonas, the genus Diaminobutyricmonas, the genus Bosea, the genus Shinella, or Fungi, examples thereof include, but are not limited to, Pseudomonas fluorescens, Pseudomonas extremorientalis, Microbacterium oxydans, Aeromonas veronii, Diaminobutyricmonas aerilata, Bosea robinae, Shinella curvata, Fungi, Pseudomons koreensis, and Aeromonas media, and the microorganism may be a new species.

In one embodiment, the microorganism derived from Japanese rice fish of the present disclosure has an ability to degrade at least one selected from the group consisting of cellulose, hemicellulose, and lignin, and may be a microorganism such as Pseudomonas fluorescens whose nucleic acid sequence of 16S rRNA is SEQ ID NO: 4, Pseudomonas fluorescens whose nucleic acid sequence of 16S rRNA is SEQ ID NO: 5, Pseudomonas extremorientalis whose nucleic acid sequence of 16S rRNA is SEQ ID NO: 6, Microbacterium oxydans whose nucleic acid sequence of 16S rRNA is SEQ ID NO: 7, Aeromonas veronii whose nucleic acid sequence of 16S rRNA is SEQ ID NO: 8, Diaminobutyricmonas aerilata whose nucleic acid sequence of 16S rRNA is SEQ ID NO: 9, Bosea robinae whose nucleic acid sequence of 16S rRNA is SEQ ID NO: 10, Aeromonas veronii whose nucleic acid sequence of 16S rRNA is SEQ ID NO: 11, Pseudomons koreensis whose nucleic acid sequence of 16S rRNA is SEQ ID NO: 12, Aeromonas media whose nucleic acid sequence of 16S rRNA is SEQ ID NO: 13, or Pseudomonas fluorescens whose nucleic acid sequence of 16S rRNA is SEQ ID NO: 14.

In another embodiment, a strain derived from isaza other than GI35, a strain derived from yellowtail and young yellowtail, and a strain derived from fish to be cultured such as sea bream and bastard halibut may also be included.

In another embodiment, a microorganism derived from Japanese rice fish may be provided. Here, the microorganism derived from Japanese rice fish may be, for example, the genus Acidaminococcus, the genus Adlercreutzia, the genus Akkermansia, the genus Alistipes, the genus Alloscardovia, the genus Anaerococcus, the genus Anaerostipes, the genus Anaerotruncus, the genus Bacillus, the genus Bacteroides, the genus Bifidobacterium, the genus Bilophila, the genus Blautia, the genus Brachyspira, the genus Butyricicoccus, the genus Butyricimonas, the genus Campylobacter, the genus Catenibacterium, the genus Christensenella, the genus Citrobacter, the genus Clostridium, the genus Collinsella, the genus Coprobacillus, the genus Coprococcus, the genus Dehalobacterium, the genus Desulfovibrio, the genus Dialister, the genus Dorea, the genus Eggerthella, the genus Enterococcus, the genus Escherichia, the genus Faecalibacterium, the genus Finegoldia, the genus Fusobacterium, the genus Granulicatella, the genus Haemophilus, the genus Holdemania, the genus Klebsiella, the genus Lachnobacterium, the genus Lachnospira, the genus Lactobacillus, the genus Lactococcus, the genus Megamonas, the genus Megasphaera, the genus Mitsuokella, the genus Morganella, the genus Odoribacter, the genus Oscillospira, the genus Oxalobacter, the genus Parabacteroides, the genus Paraprevotella, the genus Peptostreptococcus, the genus Phascolarctobacterium, the genus Porphyromonas, the genus Prevotella, the genus Pseudomonas, the genus Pseudoramibacter Eubacterium, the genus Pyramidobacter, the genus Roseburia, the genus Ruminococcus, the genus Serratia, the genus Slackia, the genus Streptococcus, the genus Succinatimonas, the genus Sutterella, the genus Synergistes, the genus Turicibacter, the genus Veillonella, or the like.

In another embodiment, the present disclosure may provide and utilize a microorganism derived from young yellowtail. Here, the microorganism is, for example, one species of the genus Acetobacter, the genus Acidibacter, the genus Acidobacterium, the genus Acidothermus, the genus Actibacter, the genus Allorhizobium-Neorhizobium-Pararhizobium-Rhizobium, the genus Anaerococcus, the genus Anaerolinea, the genus Anaeromyxobacter, the genus Aquabacterium, the genus Aquisphaera, the genus Arenimonas, the genus Azovibrio, the genus Bacillus, the genus Bacteroides, the genus Bacteroidetes bacterium, the genus Barrientosiimonas, the genus Bdellovibrio, the genus Bellilinea, the genus Blastocatella, the genus Blastopirellula, the genus Bradyrhizobium, the genus Brevundimonas, the genus Bryobacter, the genus Caldisericum, the genus Candidatus Hepatincola, the genus Candidatus Udaeobacter, the genus Chlorobi bacterium, the genus Chryseobacterium, the genus Chthoniobacter, the genus Citreitalea, the genus Clostridium, the genus Corynebacterium, the genus Crenothrix, the genus Cutibacterium, the genus Cyanobium, the genus Dermacoccus, the genus Desulfuromonas, the genus Devosia, the genus Dinghuibacter, the genus Enhydrobacter, the genus Enterococcus, the genus Erysipelothrix, the genus Exiguobacterium, the genus Ferruginibacter, the genus Flavobacterium, the genus Fluviicola, the genus Fonticella, the genus Fusobacterium, the genus Gallionella, the genus Geobacter, the genus Geothrix, the genus Halomonas, the genus Hydrogenophaga, the genus Hydrogenophilus, the genus Hyphomicrobium, the genus Ignavibacterium, the genus Immundisolibacter, the genus Kocuria, the genus Lacihabitans, the genus Lactobacillus, the genus Lactococcus, the genus Lawsonella, the genus Legionella, the genus Leptolinea, the genus Limnobacter, the genus Longilinea, the genus Luteolibacter, the genus Massilia, the genus Methylobacter, the genus Methylocystis, the genus Methylotenera, the genus Microbacterium, the genus Mycobacterium, the genus Novosphingobium, the genus Paludibaculum, the genus Paracoccus, the genus Paraperlucidibaca, the genus Pedomicrobium, the genus Phaeodactylibacter, the genus Phreatobacter, the genus Piscinibacter, the genus Porphyrobacter, the genus Prasinophyceae, the genus Prevotella, the genus Prosthecobacter, the genus Proteiniphilum, the genus Pseudohongiella, the genus Pseudomonas, the genus Pseudonocardia, the genus Pseudorhodobacter, the genus Rhodobacter, the genus Rhodomicrobium, the genus Rickettsiella, the genus Roseiarcus, the genus Roseomonas, the genus Rubripirellula, the genus Ruminiclostridium, the genus Shewanella, the genus Soehngenia, the genus Solibacter, the genus Sphaerochaeta, the genus Sphingobacteriales, the genus Sphingomonas, the genus Spirochaeta, the genus Staphylococcus, the genus Sulfuritalea, the genus Synechococcus, the genus Taibaiella, the genus Tenacibaculum, the genus Terrimonas, the genus Thermomonas, the genus Treponema, the genus Trichodesmium, and the genus Williamsia.

In still another embodiment, the present disclosure provides a microorganism derived from isaza. Here, the microorganism may be, for example, one species of the genus Shewanella, the genus Bacillus, the genus Aeromonas, and the genus Psychrobacter, and more particularly, may be, for example, Shewanella baltica, Bacillus marisflavi, Aeromonas veronii, Psychrobacter faecalis, or Psychrobacter alimentarius.

In one embodiment, a novel use of an intestinal microflora-derived microorganism is provided, focusing on the use of the present disclosure. The present disclosure provides a composition for use in a method for producing an improved target organism, the composition containing an exogenous microorganism or a part thereof, in which the method includes: a step A) of selecting an individual exhibiting the improvement from candidate microorganisms of an organism species to which a derived individual different from the target individual belongs; a step B) of obtaining, in a derived individual exhibiting the improvement, an exogenous microorganism involved in the improvement or a part thereof from a gastrointestinal microflora of the derived individual; a step C) of introducing the exogenous microorganism or a part thereof into the target individual; and a step D) of optionally confirming the properties of the gastrointestinal microflora in the target individual, and confirming that a desired improvement is achieved. It is understood that any embodiment described elsewhere in the present specification can be employed for each of these steps.

In one embodiment, the step C) may include a step of introducing the exogenous microorganism or a part thereof into the target individual at least at a part of a time during breeding of the target individual. In one embodiment, the step C) may include a step of introducing the exogenous microorganism or a part thereof into the target individual at least at a part of a time during breeding of the target individual, and breeding the target individual without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks.

In one embodiment, the step C) may include a step of introducing the exogenous microorganism or a part thereof into the target individual at least at a part of a time during growing of the target individual. In one embodiment, the step C) may include a step of introducing the exogenous microorganism or a part thereof into the target individual at least at a part of a time during growing of the target individual, and breeding the target individual without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks. In some embodiments, the growth time can be any period of time during which the body length increases, and in some embodiments, the growth time can be any period of time after beginning of postpartum feed or water ingestion, and can be 1 day, 2 days, 3 days, 4 days, 5 days, 6 days, 7 days, 8 days, 9 days, 10 days, 2 weeks, 3 weeks, or 4 weeks after beginning of postpartum feed or water ingestion. The exogenous microorganism or a part thereof is introduced into the target individual during the time, such that the exogenous microorganism or a part thereof can be stably maintained in the bacterial flora in the body of the target individual.

In one embodiment, the method of the present invention may further include a step of optionally confirming that a desired improvement is achieved in the target individual. In a certain embodiment, the desired improvement may be a change (increase or decrease) in body weight, a change in intestinal bacterial flora, a change (increase or decrease) in fat composition in the body, a change (increase or decrease) in fat percentage in body weight, an increase in intake, or an improvement in digestion and absorption of plant feed.

In one embodiment, the method of the present invention may further include a step B′) of selecting an exogenous microorganism suitable (“compatible”) for the target individual, or a part thereof, and a step C′) of introducing the suitable exogenous microorganism or a part thereof into the target individual. In a certain embodiment, the exogenous microorganism or a part thereof suitable for the target individual may be an exogenous microorganism or a part thereof that results in a desired improvement or is susceptible to engraftment in the target individual. In some embodiments, when the target individual is an individual living in seawater, the exogenous microorganism or a part thereof suitable for the target individual may be an exogenous microorganism derived from an organism living in seawater or a part thereof. In some embodiments, when the target individual is an individual living in freshwater, the exogenous microorganism or a part thereof suitable for the target individual may be an exogenous microorganism derived from an organism living in freshwater or a part thereof. In some embodiments, when the target individual is an individual living in brackish water, the exogenous microorganism or a part thereof suitable for the target individual may be an exogenous microorganism derived from an organism living in brackish water or a part thereof.

The present disclosure provides a method for breeding an individual using the method described in the present disclosure.

In another aspect, there is provided an exogenous microorganism for use in the method of the present disclosure or a part thereof, or an individual containing the same.

### (Modified Organism)

The present disclosure provides an individual of an organism belonging to an organism species having a gastrointestinal tract, the individual containing an exogenous microorganism derived from a gastrointestinal tract of a derived individual different from the individual, or a part thereof.

In one embodiment, a microflora in the gastrointestinal tract of the individual of the present disclosure is different from a naturally occurring microflora.

In another embodiment, the bacterial flora in the gastrointestinal tract of the individual of the present disclosure is characterized in that although the diversity index (Shannon index or the like) of the metagenomic analysis result is decreased, the microflora that contributes to digestion and absorption of nutrients (carbohydrate degradation and amino acid metabolism) is increased, and can be characterized by, for example, the presence of certain bacterial species, a variation in diversity index, and the like.

### (Product)

In another aspect, the present disclosure provides a product produced by the individual of the present disclosure.

The product provided by the present disclosure is selected from meat, an internal organ, milk, egg, and alcohol, but is not limited thereto. The product includes meat which is a direct product derived from an intestinal microorganism-modified organism.

In another aspect, the present disclosure provides a processed product obtained by processing the product of the present disclosure. Such a processed product may be a meat processed product, a dairy product, or the like.

### (Exogenous Microorganism or Part Thereof)

In another aspect, the present invention relates to a microorganism for a modified organism, and provides an exogenous microorganism having an ability to convert a component of a photosynthetic organism, such as a plan, that is not a nutrient source in a useful animal into a nutrient source in the animal, or a part thereof.

### (Useful Strain)

In one aspect, the present disclosure provides a novel microorganism derived from Japanese rice fish having an ability to degrade at least one selected from the group consisting of cellulose, hemicellulose, and lignin. The microorganism may be, for example, a species of the genus Pseudomonas, the genus Microbacterium, the genus Aeromonas, the genus Diaminobutyricmonas, the genus Bosea, the genus Shinella, or Fungi, examples thereof include, but are not limited to, Pseudomonas fluorescens, Pseudomonas extremorientalis, Microbacterium oxydans, Aeromonas veronii, Diaminobutyricmonas aerilata, Bosea robinae, Shinella curvata, Fungi, Pseudomons koreensis, and Aeromonas media, and the microorganism may be a new species.

In one embodiment, the microorganism derived from Japanese rice fish of the present disclosure has an ability to degrade at least one selected from the group consisting of cellulose, hemicellulose, and lignin, may be more specifically a microorganism such as Pseudomonas sp. whose nucleic acid sequence of 16S rRNA is SEQ ID NO: 4, Pseudomonas sp. whose nucleic acid sequence of 16S rRNA is SEQ ID NO: 5, Pseudomonas sp. whose nucleic acid sequence of 16S rRNA is SEQ ID NO: 6, Microbacterium sp. whose nucleic acid sequence of 16S rRNA is SEQ ID NO: 7, Aeromonas sp. whose nucleic acid sequence of 16S rRNA is SEQ ID NO: 8, Diaminobutyricmonas sp. whose nucleic acid sequence of 16S rRNA is SEQ ID NO: 9, Bosea sp. whose nucleic acid sequence of 16S rRNA is SEQ ID NO: 10, Aeromonas sp. whose nucleic acid sequence of 16S rRNA is SEQ ID NO: 11, Pseudomons sp. whose nucleic acid sequence of 16S rRNA is SEQ ID NO: 12, Aeromonas sp. whose nucleic acid sequence of 16S rRNA is SEQ ID NO: 13, or Pseudomonas sp. whose nucleic acid sequence of 16S rRNA is SEQ ID NO: 14, or more particularly a microorganism such as Pseudomonas sp. whose nucleic acid sequence of 16S rRNA is SEQ ID NO: 4, Pseudomonas sp. whose nucleic acid sequence of 16S rRNA is SEQ ID NO: 5, Pseudomonas sp. whose nucleic acid sequence of 16S rRNA is SEQ ID NO: 6, Microbacterium sp. whose nucleic acid sequence of 16S rRNA is SEQ ID NO: 7, Aeromonas sp. whose nucleic acid sequence of 16S rRNA is SEQ ID NO: 8, Diaminobutyricmonas sp. whose nucleic acid sequence of 16S rRNA is SEQ ID NO: 9, Bosea sp. whose nucleic acid sequence of 16S rRNA is SEQ ID NO: 10, Aeromonas sp. whose nucleic acid sequence of 16S rRNA is SEQ ID NO: 11, Pseudomons sp. whose nucleic acid sequence of 16S rRNA is SEQ ID NO: 12, Aeromonas sp. whose nucleic acid sequence of 16S rRNA is SEQ ID NO: 13, or Pseudomonas sp. whose nucleic acid sequence of 16S rRNA is SEQ ID NO: 14, and still more particularly, may be a microorganism such as Pseudomonas fluorescens whose nucleic acid sequence of 16S rRNA is SEQ ID NO: 4, Pseudomonas extremorientalis whose nucleic acid sequence of 16S rRNA is SEQ ID NO: 5, Pseudomonas fluorescens whose nucleic acid sequence of 16S rRNA is SEQ ID NO: 6, Microbacterium oxydans whose nucleic acid sequence of 16S rRNA is SEQ ID NO: 7, Aeromonas veronii whose nucleic acid sequence of 16S rRNA is SEQ ID NO: 8, Diaminobutyricmonas aerilata whose nucleic acid sequence of 16S rRNA is SEQ ID NO: 9, Bosea robinae whose nucleic acid sequence of 16S rRNA is SEQ ID NO: 10, Aeromonas veronii whose nucleic acid sequence of 16S rRNA is SEQ ID NO: 11, Pseudomons koreensis whose nucleic acid sequence of 16S rRNA is SEQ ID NO: 12, Aeromonas media whose nucleic acid sequence of 16S rRNA is SEQ ID NO: 13, or Pseudomonas fluorescens whose nucleic acid sequence of 16S rRNA is SEQ ID NO: 14.

In another embodiment, a strain derived from isaza other than GI35, a strain derived from yellowtail and young yellowtail, and a strain derived from fish to be cultured such as sea bream and bastard halibut may also be included.

In another embodiment, a microorganism derived from Japanese rice fish may be provided. Here, the microorganism derived from Japanese rice fish may be, for example, the genus Acidaminococcus, the genus Adlercreutzia, the genus Akkermansia, the genus Alistipes, the genus Alloscardovia, the genus Anaerococcus, the genus Anaerostipes, the genus Anaerotruncus, the genus Bacillus, the genus Bacteroides, the genus Bifidobacterium, the genus Bilophila, the genus Blautia, the genus Brachyspira, the genus Butyricicoccus, the genus Butyricimonas, the genus Campylobacter, the genus Catenibacterium, the genus Christensenella, the genus Citrobacter, the genus Clostridium, the genus Collinsella, the genus Coprobacillus, the genus Coprococcus, the genus Dehalobacterium, the genus Desulfovibrio, the genus Dialister, the genus Dorea, the genus Eggerthella, the genus Enterococcus, the genus Escherichia, the genus Faecalibacterium, the genus Finegoldia, the genus Fusobacterium, the genus Granulicatella, the genus Haemophilus, the genus Holdemania, the genus Klebsiella, the genus Lachnobacterium, the genus Lachnospira, the genus Lactobacillus, the genus Lactococcus, the genus Megamonas, the genus Megasphaera, the genus Mitsuokella, the genus Morganella, the genus Odoribacter, the genus Oscillospira, the genus Oxalobacter, the genus Parabacteroides, the genus Paraprevotella, the genus Peptostreptococcus, the genus Phascolarctobacterium, the genus Porphyromonas, the genus Prevotella, the genus Pseudomonas, the genus Pseudoramibacter Eubacterium, the genus Pyramidobacter, the genus Roseburia, the genus Ruminococcus, the genus Serratia, the genus Slackia, the genus Streptococcus, the genus Succinatimonas, the genus Sutterella, the genus Synergistes, the genus Turicibacter, the genus Veillonella, or the like. These genera are based on metagenomic analysis and may vary.

In another embodiment, the present disclosure may provide and utilize a microorganism derived from young yellowtail. Here, the microorganism is, for example, one species of the genus Acetobacter, the genus Acidibacter, the genus Acidobacterium, the genus Acidothermus, the genus Actibacter, the genus Allorhizobium-Neorhizobium-Pararhizobium-Rhizobium, the genus Anaerococcus, the genus Anaerolinea, the genus Anaeromyxobacter, the genus Aquabacterium, the genus Aquisphaera, the genus Arenimonas, the genus Azovibrio, the genus Bacillus, the genus Bacteroides, the genus Bacteroidetes bacterium, the genus Barrientosiimonas, the genus Bdellovibrio, the genus Bellilinea, the genus Blastocatella, the genus Blastopirellula, the genus Bradyrhizobium, the genus Brevundimonas, the genus Bryobacter, the genus Caldisericum, the genus Candidatus Hepatincola, the genus Candidatus Udaeobacter, the genus Chlorobi bacterium, the genus Chryseobacterium, the genus Chthoniobacter, the genus Citreitalea, the genus Clostridium, the genus Corynebacterium, the genus Crenothrix, the genus Cutibacterium, the genus Cyanobium, the genus Dermacoccus, the genus Desulfuromonas, the genus Devosia, the genus Dinghuibacter, the genus Enhydrobacter, the genus Enterococcus, the genus Erysipelothrix, the genus Exiguobacterium, the genus Ferruginibacter, the genus Flavobacterium, the genus Fluviicola, the genus Fonticella, the genus Fusobacterium, the genus Gallionella, the genus Geobacter, the genus Geothrix, the genus Halomonas, the genus Hydrogenophaga, the genus Hydrogenophilus, the genus Hyphomicrobium, the genus Ignavibacterium, the genus Immundisolibacter, the genus Kocuria, the genus Lacihabitans, the genus Lactobacillus, the genus Lactococcus, the genus Lawsonella, the genus Legionella, the genus Leptolinea, the genus Limnobacter, the genus Longilinea, the genus Luteolibacter, the genus Massilia, the genus Methylobacter, the genus Methylocystis, the genus Methylotenera, the genus Microbacterium, the genus Mycobacterium, the genus Novosphingobium, the genus Paludibaculum, the genus Paracoccus, the genus Paraperlucidibaca, the genus Pedomicrobium, the genus Phaeodactylibacter, the genus Phreatobacter, the genus Piscinibacter, the genus Porphyrobacter, the genus Prasinophyceae, the genus Prevotella, the genus Prosthecobacter, the genus Proteiniphilum, the genus Pseudohongiella, the genus Pseudomonas, the genus Pseudonocardia, the genus Pseudorhodobacter, the genus Rhodobacter, the genus Rhodomicrobium, the genus Rickettsiella, the genus Roseiarcus, the genus Roseomonas, the genus Rubripirellula, the genus Ruminiclostridium, the genus Shewanella, the genus Soehngenia, the genus Solibacter, the genus Sphaerochaeta, the genus Sphingobacteriales, the genus Sphingomonas, the genus Spirochaeta, the genus Staphylococcus, the genus Sulfuritalea, the genus Synechococcus, the genus Taibaiella, the genus Tenacibaculum, the genus Terrimonas, the genus Thermomonas, the genus Treponema, the genus Trichodesmium, and the genus Williamsia. These genera are based on metagenomic analysis and may vary.

In still another embodiment, the present disclosure provides a microorganism derived from isaza. Here, the microorganism may be, for example, one species of the genus Shewanella, the genus Bacillus, the genus Aeromonas, and the genus Psychrobacter, and more particularly, may be, for example, Shewanella baltica, Bacillus marisflavi, Aeromonas veronii, Psychrobacter faecalis, or Psychrobacter alimentarius.

### (Application of Synthesis of Polyunsaturated Fatty Acid)

In another aspect, the present disclosure provides a composition for producing a fatty acid other than EPA, which contains GI35 strain or a microorganism having an ability equivalent to that of the GI35 strain, and a use, method, and related technique thereof.

In another aspect, the present disclosure provides a composition for producing a fatty acid other than EPA, which contains an unsaturated fatty acid synthase group derived from GI35 strain or a synthase group having an ability equivalent to that of the unsaturated fatty acid synthase group, and a use, method, and related technique thereof.

In one embodiment, in the composition of the present disclosure, the synthase group contains an extract of the GI35 strain.

In another embodiment, the fatty acid other than EPA in the present disclosure may be palmitoleic acid, oleic acid, linoleic acid, γ-linolenic acid, α-linolenic acid, stearidonic acid, dihomo-γ-linolenic acid, arachidonic acid, eicosatetraenoic acid (ETA), osbond acid, docosapentaenoic acid (DPA), or docosahexaenoic acid (DHA), and may be other fatty acids.

### (Provided Form of Microorganism)

In the present specification, the microorganism of the present disclosure or a part thereof may be provided in the form of various compositions, which may be feed. In the present disclosure, the shape of the feed is not particularly limited, and in one embodiment, the feed may have the same shape as a known animal feed. Examples of the shape of the feed of the present disclosure include, but are not limited to, a moist pellet, a dry pellet, a powder, a crumble, and a kneaded feed. The feed of the present disclosure can be produced by adding or mixing the exogenous microorganism of the present disclosure or a part thereof to or with a raw material of feed for an animal, in a production process of feed for an animal, or to a feed product for an animal. The methods such as addition and mixing of the exogenous microorganism of the present disclosure or a part thereof are known. By culturing the exogenous microorganism of the present disclosure or a part thereof, a required amount of a microorganism can be obtained. Culturing the exogenous microorganism of the present disclosure or a part thereof will be described below The exogenous microorganism of the present disclosure obtained by culture or a part thereof can be separated from the medium by a method such as centrifugation. The obtained exogenous microorganism of the present disclosure or a part thereof can also be dried by a method such as lyophilization. A lyophilized product of the exogenous microorganism of the present disclosure or a part thereof or a culture solution of the exogenous microorganism of the present disclosure or a part thereof may be mixed in the production process of the animal feed. Alternatively, the resulting animal feed may be impregnated with a culture solution of the exogenous microorganism of the present disclosure or a part thereof, or may be sprinkled with a lyophilized product of the exogenous microorganism of the present disclosure or a part thereof. The feed of the present disclosure is produced so that all or a part of the exogenous microorganism of the present disclosure or a part thereof in the feed can reach the gastrointestinal tract, such as the intestines, of the animal in a living state.

A blending amount of the exogenous microorganism of the present disclosure or a part thereof in the feed can be appropriately changed according to the type and size of the animal, the components in the feed, and the like. A dosage of the feed containing the exogenous microorganism of the present disclosure or a part thereof can also be appropriately changed according to the type and size of the animal. In one example, the dosage of the feed containing the exogenous microorganism of the present disclosure or a portion thereof may be similar to normal feed.

The feed of the present disclosure may be used in combination with another feed.

Fish meal contains EPA and/or other fatty acids, DHA, or the like, and fish meal is blended in culture feed. However, since the catch of Japanese pilchard, which is a raw material of fish meal, is significantly reduced, it is difficult to add fish meal to culture feed, and the amount has to be reduced. Therefore, fish meal substitute feed mainly containing soybean and corn has been developed. However, raw materials of the photosynthetic organism such as plant raw materials do not contain EPA and/or other fatty acids or essential fatty acids such as DHA. Therefore, the above problems can be solved by mixing the exogenous microorganism of the present disclosure or a part thereof with a plant raw material to produce and use the feed of the present disclosure. In addition, when salmonid fish seedlings grown in an EPA and/or other fatty acid/DHA-enhanced diet are released into a river, an increase in the mortality of immature fish due to deficiency of EPA and/or other fatty acids causes a decrease in catch. Under such circumstances, when the feed of the present disclosure is administered to fry of salmonid fish, EPA and/or other fatty acids are continuously and stably produced in the fry even when the fry are released into a river, the mortality can be reduced, and the decrease in catch can be stopped.

As shown in Examples, the exogenous microorganism of the present disclosure or a part thereof well survives in the intestinal tract of fish and becomes continuously present in the intestinal tract. Therefore, the exogenous microorganism of the present disclosure or a part thereof is administered to fish, such that fish in which the exogenous microorganism of the present disclosure or a part thereof is present in the intestinal tract can be obtained. A method for administering the exogenous microorganism of the present disclosure or a part thereof to fish may be any method, but is not particularly limited, and in general, the exogenous microorganism of the present disclosure or a part thereof is mixed with feed and administered. Fish in which the exogenous microorganism of the present disclosure or a part thereof is present in the intestinal tract can produce EPA and/or other fatty acids continuously and stably in the body.

Therefore, in a further aspect, the present disclosure provides a method for producing fish in which the exogenous microorganism of the present disclosure or a part thereof is present in a gastrointestinal tract such as an intestinal tract, the method including administering the exogenous microorganism of the present disclosure or a part thereof to fish.

In a further aspect, the present disclosure provides a method for producing fish that produces EPA and/or other fatty acids in a body, the method including administering the exogenous microorganism of the present disclosure or a part thereof to fish.

The administration in the invention of these aspects may be performed by administering the feed.

In a further aspect, the present disclosure provides fish in which the exogenous microorganism of the present disclosure or a part thereof is present in a gastrointestinal tract such as an intestinal tract (excluding isaza in which the exogenous microorganism of the present disclosure or a part thereof is present in an intestinal tract), and fish in which the exogenous microorganism of the present disclosure or a part thereof is present in a gastrointestinal tract such as an intestinal tract and produces EPA and/or other fatty acids in the body (excluding isaza in which the exogenous microorganism of the present disclosure or a part thereof is present in an intestinal tract). These fish can produce EPA and/or other fatty acids continuously and stably in the body, and their meat also has a high EPA and/or other fatty acid content.

A seawater fish and a diadromous fish cannot produce EPA and/or other fatty acids by themselves. A freshwater fish can only self-produce small amounts of EPA and/or other fatty acids. In contrast, fish to which the feed of the present disclosure is administered can produce EPA and/or other fatty acids continuously and stably in all the bodies of the seawater fish, the diadromous fish, and the freshwater fish. That is, the feed of the present disclosure is administered, such that fish rich in EPA and/or other fatty acids can be obtained continuously and stably. Eating fish rich in EPA and/or other fatty acids is expected to maintain and enhance health, leading to prevention of a cardiovascular disease, a lifestyle disease, or the like.

In a further aspect, the present disclosure provides a method for producing enhanced growth of fish, the method including administering the exogenous microorganism of the present disclosure or a part thereof to fish. By administering the exogenous microorganism of the present disclosure or a part thereof to fish, the growth of fish can be promoted. For example, the exogenous microorganism of the present disclosure or a part thereof may be administered throughout a fry period or may be administered transiently during the fry period. The fish obtained by the method of the aspect may be fish rich in EPA and/or other fatty acids.

In a further aspect, the present disclosure provides a method for producing fish in which a microflora (for example, a non-viral microflora, a germ flora, a bacterial flora, or the like) in a gastrointestinal tract (for example, intestines) is modified, the method including administering the exogenous microorganism of the present disclosure or a part thereof to fish. The exogenous microorganism of the present disclosure or a part thereof is administered to fish, such that the microflora (for example, a non-viral microflora, a germ flora, a bacterial flora, or the like) in the gastrointestinal tract (for example, intestines) after growth can be modified. Administration of the exogenous microorganism of the present disclosure or a part thereof is as described above. By modifying the gastrointestinal microflora using the method of the aspect, the activity of various enzymes of the gastrointestinal microflora can be enhanced or inhibited. For example, the activity of an enzyme related to promotion of digestion and absorption of food may be enhanced, or the activity of an enzyme that contributes to improvement of meat quality may be enhanced. The fish obtained by the method of the aspect may be fish rich in EPA and/or other fatty acids.

In a further aspect, the present disclosure provides a method for producing EPA and/or other fatty acids, the method including culturing the exogenous microorganism of the present disclosure or a part thereof.

A method for culturing the exogenous microorganism of the present disclosure or a part thereof may be any culturing method as long as the exogenous microorganism of the present disclosure or a part thereof can proliferate to produce EPA and/or other fatty acids. The exogenous microorganism of the present disclosure or a part thereof may be cultured by the method similar to a known method for culturing bacteria. For example, the exogenous microorganism of the present disclosure or a part thereof may be cultured in a medium containing glucose, peptone, yeast extract, saline, and other inorganic salts. The medium may be either a liquid medium or a solid medium. In the case of liquid culture, shaking culture, stirring culture, static culture, or the like may be used. As a culture vessel, a flask, ajar, a tank, or the like may be used. The exogenous microorganism of the present disclosure or a part thereof is capable of proliferating at about 4° C. to about 37° C., and preferably proliferating at about 18° C. to about 30° C. On the other hand, a preferred culture temperature compatible with a sufficiently high EPA and/or other fatty acid production amount is about 4° C. to about 20° C. Those skilled in the art can select and determine suitable culture conditions for the exogenous microorganism of the present disclosure or a part thereof. The amount of EPA and/or other fatty acids in the medium can be determined, for example, using gas chromatography. The produced EPA and/or other fatty acids can be recovered from the culture solution or cells by known methods.

A method for preserving the exogenous microorganism of the present disclosure or a part thereof may be similar to a known method for preserving bacteria. The preservation method includes, but is not limited to, preservation in slant, lyophilization, and the like.

In still another aspect, the present disclosure provides a method for producing EPA and/or other fatty acids, the method including culturing a host cell into which a gene group related to EPA and/or other fatty acid production of the exogenous microorganism of the present disclosure or a part thereof or a variant of the gene group is introduced.

The present inventors perform whole genome cloning of a gene group pfa operon involved in EPA and/or other fatty acid production of the exogenous microorganism of the present disclosure or a part thereof. EPA and/or other fatty acids can be produced by incorporating the operon into an expression vector to introduce the vector into a host cell (for example, E. coli) and culture the host cell. Each component of the pfa operon may be incorporated into a separate expression vector for use. Various expression vectors and host cells that can be used in the method are known, and can be appropriately selected and used.

Examples of a gene group related to EPA and/or other fatty acid production of the exogenous microorganism of the present disclosure that can be used in the method for producing EPA and/or other fatty acids of the present disclosure or a part thereof include those having the base sequence set forth in SEQ ID NO: 1 (pfa operon). The gene group related to EPA and/or other fatty acid production of the exogenous microorganism of the present disclosure or a part thereof includes five genes pfaA, pfaB, pfaC, pfaD and pfaE. The nucleotide sequence of pfaA is set forth in the nucleotide sequence at positions 2413 to 10503 of SEQ ID NO: 1. The nucleotide sequence of pfaB is set forth in the nucleotide sequence at positions 10500 to 12794 of SEQ ID NO: 1. The nucleotide sequence of pfaC is set forth in the nucleotide sequence at positions 12791 to 18724 of SEQ ID NO: 1. The nucleotide sequence of pfaD is set forth in the nucleotide sequence at positions 18835 to 20481 of SEQ ID NO: 1. The complementary strand sequence of the nucleotide sequence of pfaE is set forth in the nucleotide sequence at positions 30 to 899 of SEQ ID NO: 1. A variant of the gene group related to EPA and/or other fatty acid production of the exogenous microorganism of the disclosure or a part thereof may include genes corresponding to pfaA, pfaB, pfaC, pfaD, and pfaE of the exogenous microorganism of the disclosure or a part thereof. The base sequences of the genes corresponding to pfaA, pfaB, pfaC, pfaD and pfaE may have a homology of 70% or more, preferably 80% or more, more preferably 90% or more, still more preferably 95% or more, and most preferably 98% or more, respectively, with respect to the base sequences of pfaA, pfaB, pfaC, pfaD and pfaE of PI35 strain (here, the case where all the five genes have a homology of 100% is excluded). In addition, the variant of the gene group related to EPA and/or other fatty acid production of the exogenous microorganism of the present disclosure or a part thereof may have a homology of 70% or more, preferably 80% or more, more preferably 90% or more, still more preferably 95% or more, and most preferably 98% or more, with respect to the base sequence set forth in SEQ ID NO: 1. A sequence homology between genes can be examined using known programs such as FASTA and BLAST. In addition, the variant of the gene group related to EPA and/or other fatty acid production of the exogenous microorganism of the disclosure or a part thereof may have a base sequence corresponding to the base sequence shown in SEQ ID NO: 1 of the variant strain of the exogenous microorganism of the present disclosure or a part thereof. However, the variant of the gene group related to EPA and/or other fatty acid production of the exogenous microorganism of the present disclosure or a part thereof produces 70% or more, preferably 80% or more, more preferably 90% or more, still more preferably 100% or more, and most preferably 120% or more, of EPA and/or other fatty acids, compared to the case of using the gene group related to EPA and/or other fatty acid production of the exogenous microorganism of the present disclosure or a part thereof. The variant of the gene group related to EPA and/or other fatty acid production of the exogenous microorganism of the present disclosure or a part thereof may be produced by known methods such as genetic recombination, such as site-specific mutagenesis, genome editing, and chemical methods.

The introduction of the gene group related to EPA and/or other fatty acid production into a host cell is usually performed by introducing an expression vector incorporating the gene group into cells. The type of the expression vector, the method for incorporating the gene group into the expression vector, and the method for introducing the gene group into the expression vector are known, and can be appropriately selected according to the type of the host cell, the size of the introduced gene, the base sequence, and the like. pfaA, pfaB, pfaC, pfaD, and pfaE may all be incorporated into one expression vector and introduced into a host cell, or may be divided into a plurality of expression vectors and incorporated, and these vectors may be introduced into cells.

In still another aspect, the present disclosure provides cells into which a gene group related to EPA and/or other fatty acid production of the exogenous microorganism of the present disclosure or a part thereof or a variant of the gene group is introduced. Such cells can be cultured to produce EPA and/or other fatty acids. The cell may be any cell of a microbial cell, an animal cell, and a plant cell, but is not particularly limited, and typical examples thereof include bacterial cells such as E. coli cells and Bacillus subtilis cells.

In still another embodiment, the present disclosure provides food and drink containing the exogenous microorganism of the present disclosure or a part thereof. Food and drink includes foods, beverages, and health foods such as supplements and so-called FOSHU. By ingesting the food and drink of the present disclosure, the exogenous microorganism of the present disclosure or a part thereof remains viable in the intestinal tract and is continuously present in the intestinal tract, and EPA and/or other fatty acids are continuously produced in the body. This is expected to maintain and improve health, leading to prevention of a cardiovascular disease, a lifestyle disease, or the like. Specifically, it is expected to have effects such as lowering a neutral fat level and inhibiting platelet aggregation. Since the exogenous microorganism of the present disclosure or a part thereof is a germ that inhabits in the intestinal tract of isaza, which is edible, feed or food and drink containing the exogenous microorganism or a part thereof is highly safe.

The food and drink of the present disclosure can be produced by adding or mixing the exogenous microorganism of the present disclosure or a part thereof to a raw material of the food and drink, in a production process of the food and drink, or to a food and drink product. A lyophilized product of the exogenous microorganism of the present disclosure or a part thereof or a culture solution of the exogenous microorganism of the present disclosure or a part thereof may be mixed in the production process of the food and drink. Alternatively, the resulting food and drink may be impregnated with a culture solution of the exogenous microorganism of the present disclosure or a part thereof, or may be sprinkled with a lyophilized product of the exogenous microorganism of the present disclosure or a part thereof. The food and drink of the present disclosure is produced so that all or a part of the exogenous microorganism of the present disclosure or a part thereof in the food and drink can reach a gastrointestinal tract of an organism in a living state. Supplements and health foods may be produced by a method similar to or equivalent to a known manufacture of pharmaceuticals.

The shape of the food and drink of the present disclosure may be any shape, and may be, for example, a shape similar to that of an existing food and drink, or may be a shape such as a drink, a paste, a cream, a tablet, a powder, a granule, or a capsule. In addition, the food and drink of the present disclosure may be used as a food additive.

As described above, since the food and drink of the present disclosure is highly safe, the amount of the food and drink ingested of the present disclosure is not particularly limited.

In still another embodiment, the present disclosure provides food and drink obtained by processing fish in which the exogenous microorganism of the present disclosure or a part thereof is present in a gastrointestinal tract (for example, an intestinal tract) (excluding isaza in which the exogenous microorganism of the present disclosure or a part thereof is present in an intestinal tract), or fish in which the exogenous microorganism of the present disclosure or a part thereof is present in a gastrointestinal tract (for example, an intestinal tract) and produces EPA in the body (excluding isaza in which the exogenous microorganism of the present disclosure or a part thereof is present in an intestinal tract). These types of fish are rich in EPA. Therefore, the food and drink obtained by processing these types of fish also abundantly contains EPA, and it is expected that the amount of EPA ingested is increased by ingesting these types of fish, and health is maintained and enhanced, leading to prevention of a cardiovascular disease, a lifestyle disease, or the like. Specifically, it is expected to have effects such as lowering a neutral fat level and inhibiting platelet aggregation.

The food and drink obtained by processing fish also includes foods, beverages, and health foods such as supplements and so-called FOSHU. The shape of the food and drink obtained by processing fish may be any shape. The food and drink obtained by processing fish may be one obtained by cooking (for example, boiling, grilling, steaming, preparing raw fish, or the like) the whole or a part of the fish according to a normal cooking method, or one obtained by mixing the whole or a part of the fish with another food material. Alternatively, the food and drink obtained by processing fish may be an extract of the whole or a part of the fish (for example, the shape of a capsule encapsulating an extract), or may be a product obtained by drying the whole or a part of the fish to form powder, granules, tablets, flakes, or the like. The amount of the food and drink ingested, which is obtained by processing fish, is not particularly limited.

### (Pharmaceuticals)

The present disclosure may itself be in the form of food and drink, feed, pharmaceuticals, or the like, or may be in the form of being contained in food and drink, pharmaceutical, or the like as an additive. An ingestion (administration) route of the composition of the present disclosure may be either oral or parenteral, but is usually oral. In addition, examples of the parenteral ingestion (administration) include rectal administration.

Note that the bacteria specified by the bacterial names exemplified above are not limited to the strains themselves deposited or registered in a predetermined institution under the bacterial names (hereinafter, for convenience of description, it is also referred to as a “deposited strain”), and include strains substantially equivalent thereto (also referred to as “derived strains” or “induced strains”). That is, the present invention is not limited to the strain deposited in the depository institution with the accession number, and includes strains substantially equivalent thereto. For each bacterium, the “strain substantially equivalent to the deposited strain” refers to a strain that belongs to the same species as the deposited strain, has an effect of improving the intestinal bacterial flora, and has a base sequence of its 16S rRNA gene having preferably 99.86% or more, more preferably 99.93% or more, and still more preferably 100% identity with the base sequence of the 16S rRNA gene of the deposited strain, and preferably refers to a strain having the same bacteriological properties as those of the deposited strain. For each bacterium, a strain substantially equivalent to the deposited strain may be, for example, a derivative strain having the deposited strain as a parent strain. Examples of the derivative strain include a strain bred from the deposited strain and a strain naturally generated from the deposited strain. Examples of the breeding method include modification by a genetic engineering technique and modification by mutation treatment. Examples of the mutation treatment include radiation with X-rays, radiation with ultraviolet rays, and treatment with a mutagen such as N-methyl-N′-nitro-N-nitrosoguanidine, ethyl methanesulfonate, or methyl methanesulfonate. Examples of the strain naturally generated from the deposited strain include a strain naturally generated during use of the deposited strain. Examples of such a strain include a variant strain which is naturally generated by culture (for example, subculture) of the deposited strain. The derivative strain may be constructed by one modification or by two or more modifications.

In the case of producing a variant strain of the microorganism of the present disclosure, the microorganism is statically cultured in MRS medium until the logarithmic proliferation phase, washed with sterile physiological saline or sterile water, and then treated with a mutagen such as N-methyl-N′-nitro-N-nitrosoguanidine (NTG) at 50 to 500 g/ml, 30 to 37° C. for 30 to 60 minutes in the sterile physiological saline or sterile water, such that a variant strain can be obtained. For induction of variation, in addition to NTG, ultraviolet rays or known mutagens such as ethyl methanesulfonic acid (EMS) and fluorouracil (5-FU) can also be used, and generally known means can be applied. The taxonomic bacteriological properties of the obtained strain can be confirmed, for example, by examining the homology of the 16S rRNA gene base sequence, examining the DNA-DNA homology by DNA-DNA hybridization with a reference strain, examining the assimilability of sugars, and the like.

Examples of an exogenous microorganism or a partially treated product thereof in the present specification include, but are not limited to, an exogenous microorganism or a partially disrupted product thereof, an exogenous microorganism or a partially extracted product thereof, and a dried product, frozen product, water dispersion, or emulsion thereof.

An exogenous microorganism disrupted product is obtained by disrupting an exogenous microorganism or a part thereof by disrupting (in this case, an exogenous microorganism or a partially disrupted product thereof is obtained), grinding, enzyme treatment, chemical treatment, dissolution, or the like, and the form of the exogenous microorganism disrupted product is arbitrary as long as the exogenous microorganism disrupted product has dual agonist activity for the peroxisome proliferator-activated receptor (PPAR) a and the peroxisome proliferator-activated receptor (PPAR) 7. For example, a liquid obtained by disrupting an exogenous microorganism or a part thereof in an aqueous medium and drying the liquid as it is by lyophilization or the like, or a liquid obtained by recovering a disrupting exogenous microorganism or a part thereof as it is (that is, essentially all the components constituting the cell) can be preferably used.

Disruption of the exogenous microorganisms or a part thereof can be performed using methods and equipment known in the art, for example, by physical disruption, enzyme lysis treatment, and the like. The physical disruption may be performed in either a wet manner (treatment in a state of an exogenous microorganism or a partial suspension thereof) or a dry manner (treatment in a state of an exogenous microorganism or a partial powder thereof), and can be performed by stirring using a homogenizer, a ball mill, a bead mill, a Dyno mill, a planetary mill, or the like, by pressure using a jet mill, a French press, a cell disruptor, or the like, or by filter filtration. The enzyme lysis treatment can disrupt a cell wall of an exogenous microorganism or a part thereof, for example, using an enzyme such as lysozyme.

Specifically, in the method for producing an exogenous microorganism disrupted product, an exogenous microorganism or a part thereof is disrupted by treating a suspension of the exogenous microorganism or a part thereof 1 to 7 times (for example, 3 to 5 times) at a peripheral speed of 10.0 to 20.0 m/s (for example, about 14.0 m/s), a treatment flow rate of 0.1 to 10 L/10 min (for example, about 1 L/10 min), and a disruption vessel temperature of 10 to 30° C. (for example, about 15° C.) using glass beads in a known DYNO-MILL cell disruptor (for example, a DYNO-MILL disruptor or the like). In addition, an exogenous microorganism or a part thereof is disrupted, for example, by treating a suspension of the exogenous microorganism or a part thereof 1 to 30 times (for example, 10 times) at a discharge pressure of 50 to 1,000 MPa (for example, 270 MPa) and a treatment flow rate of 50 to 1,000 (for example, 300 ml/min) in a known wet jet mill cell disruptor (JN20 Nano Jet Pul or the like). In addition, it is also possible to disrupt an exogenous microorganism or a part thereof by treating the exogenous microorganism or a partial powder thereof obtained from an exogenous microorganism or a part thereof in the presence of various balls (for example, 10 mm balls formed of zirconia, 5 mm balls formed of zirconia, and 1 mm balls formed of alumina) at a rotation speed of 50 to 10,000 rpm (for example, 240 rpm, 190 rpm, or 110 rpm) for 30 minutes to 20 hours (for example, 5 to 10 hours) in a known dry planetary mill cell disruptor (GOT5 Galaxy 5 or the like). An exogenous microorganism or a part thereof may be disrupted by treating an exogenous microorganism or a partial powder thereof obtained from the exogenous microorganism or a part thereof 1 to 10 times (for example, once) at a feed rate of 0.01 to 10,000 g/min (for example, 0.5 g/min) and a discharge pressure of 1 to 1,000 kg/cm² (for example, 6 kg/cm²) in a known dry jet mill cell disruptor (a jetmizer or the like).

In the present disclosure, the disrupted product of the exogenous microorganism or a part thereof exhibits an effect even when a hole is formed in the exogenous microorganism or a part thereof, and it is desirable to prepare the disrupted product so that the average major diameter of the exogenous microorganism or a part thereof is 90% or less of that before the disruption treatment. For example, when an exogenous microorganism or a part thereof is disrupted by dissolution treatment, the average major diameter of the exogenous microorganism or a part thereof may be close to 0%. Therefore, the exogenous microorganism or a part thereof can be disrupted so that the average major diameter of the exogenous microorganism or a part thereof in the exogenous microorganism or the partially disrupted product thereof is 90% or less, preferably 80% or less, 70% or less, 60% or less, or 50% or less, and more preferably 40% or less, 30% or less, or 20% or less, of the average major diameter before disruption.

The exogenous microorganism or a part thereof and/or the exogenous microorganism or the partially disrupted product thereof can be dried into a powder or granulate. A specific drying method is not particularly limited, and examples thereof include spray drying, drum drying, vacuum drying, and lyophilization, and these methods can be employed alone or in combination. In this case, a carrier or an excipient that is usually used may be added, if necessary.

Furthermore, the exogenous microorganism or the partially extracted product thereof can be obtained by performing an extraction operation from the exogenous microorganism or a part thereof or the exogenous microorganism or the partially disrupted product thereof by appropriately combining water, an organic solvent, or a mixed solvent, and recovering a fraction containing an active ingredient having a desired activity. The organic solvent is a polar solvent, a non-polar solvent, or a mixed solvent thereof, examples of the polar solvent include alcohols such as methanol, ethanol, and propanol, acetone, acetonitrile, dioxane, DMSO, and DMF, and examples of the non-polar solvent include ethers such as diethyl ether, hydrocarbons such as hexane and heptane, and alkyl halides such as dichloromethane and chloroform. In particular, it is considered that the active ingredient of the present disclosure has a property of being easily extracted with a non-polar organic solvent such as diethyl ether as described in Examples described below, and a part thereof is also extracted with a polar organic solvent such as ethanol, acetonitrile, or DMSO. The exogenous microorganism of the present disclosure or the partially extracted product thereof is also intended to encompass concentrates or residues obtained by concentration using an evaporation device such as an evaporator, and preferably removing a solvent.

Furthermore, a component or a fraction having an action of improving lipid metabolism and saccharide metabolism may be purified from the exogenous microorganism or the partially disrupted product thereof using a known separation and purification method. Examples of the separation and purification method include a method using solubility such as salt precipitation and organic solvent precipitation, a method using a difference in molecular weight such as dialysis, ultrafiltration, and gel filtration, a method using a difference in charge such as ion exchange chromatography, a method using specific binding such as affinity chromatography, and a method using hydrophobicity such as hydrophobic chromatography and reverse phase chromatography, and one of these methods or a combination of two or more thereof can be used.

The exogenous microorganism or the partially disrupted product thereof, the exogenous microorganism or the partially extracted product thereof, or the active ingredient-containing fraction obtained as described above can be used as a lipid metabolism and/or saccharide metabolism improving agent as it is or in combination with food and drink or a pharmaceutical carrier or excipient. If necessary, additives such as a disintegrant, a binder, a wetting agent, a stabilizer, a buffer, a lubricant, a preservative, a surfactant, a sweetener, a flavoring agent, a fragrance, an acidulant, and a coloring agent can be contained. In addition, a dosage form is not limited, and may be, for example, a tablet, a capsule, a granule, a triturate, a powder, a syrup, a dry syrup, a liquid, a suspension, an emulsifier, or the like.

The exogenous microorganism or a part thereof, or a treated product thereof contained in the lipid metabolism and/or saccharide metabolism improving agent of the present disclosure is produced from the number of exogenous microorganisms or parts thereof before treatment, which is the number corresponds to, but is not limited to, for example, about 10⁵ cells/g to about 10¹⁴ cells/g and preferably about 10⁸ cells/g to about 10¹² cells/g.

The lipid metabolism and/or saccharide metabolism improving agent of the present disclosure contains the exogenous microorganism or a part thereof or the exogenous microorganism or the partially treated product thereof described above as an active ingredient, and the exogenous microorganism or a part thereof or the exogenous microorganism or the partially treated product thereof may be obtained from one or a plurality of bacterial species.

The present disclosure can be provided and sold as feed labeled for health use such as development, prevention of disease, treatment, or symptom relief. Such labeling is not particularly limited, and examples thereof include “promoting development of the intestinal tract”, “improving nutrient absorption ability”, “aiding digestion”, “increasing body weight”, “promoting growth”, “inhibiting inflammation of the intestines”, “preventing intestinal diseases”, “preventing and treating infections”, “preventing and treating food poisoning”, and “preventing and treating food allergy”. The “labeling” action includes all actions for making the consumer know the above use, and any expression that can evoke and infer the above use falls under the “indication” action of the present disclosure regardless of the purpose of labeling, the content of labeling, the object or medium to be labeled, and the like.

The labeling content is preferably labeling approved by the government or the like (for example, labeling or the like approved based on various systems defined by the government and made in a manner based on such approval). In addition, it is preferable to attach such a labeling content to an advertisement material at a sales site such as a package, a container, a catalog, a brochure, or a POP advertising, other documents, and the like.

The “labeling” may include labeling of functional feed as feed, and in the case of food, labeling may be for health food, functional food, enteral nutritive food, special use food, health functional food, food for specified health use, nutritional functional food, functional labeling food, quasi-pharmaceutical product, and the like. Among them, there is a label approved by the Japanese government, for example, a label approved in a system related to food for specified health uses, nutritional functional food, or food labeled with functionality, or a system similar thereto. Specific examples thereof include a label for food for specified health use, a label for food for specified health use with conditions, a label for food with functionality, a label for effects on the structure and function of the body, a label for disease risk reduction, and a label of functionality based on scientific evidence.

### <Pharmaceutical Composition>

The present disclosure can be used as a pharmaceutical product, for example, an animal pharmaceutical product, and the like, and the composition of the present disclosure may be produced by blending a physiologically acceptable liquid or a formulation carrier.

A dosage form of the pharmaceutical composition of the present disclosure is not particularly limited, and the pharmaceutical composition can be prepared into a solid preparation such as a powder, a granule, a tablet, or a capsule; a liquid preparation such as a syrup, a suspension, or an emulsion; a suppository; an ointment; and the like. In the preparation, a preparation carrier used for normal preparation can be used. In addition, the pharmaceutical composition can also contain a component having a known or later-found intestinal development promoting action or an action of preventing, treating, or relieving symptoms of intestinal diseases.

In the present disclosure, the microorganism or the bacterial flora preferably includes at least a bacterium belonging to the genus Klebsiella, a bacterium belonging to the genus Rothia, a bacterium belonging to the genus Bifidobacterium, a bacterium belonging to the genus Enterococcus, a bacterium belonging to the genus Streptococcus, a bacterium belonging to the genus Escherichia, a bacterium belonging to the genus Staphylococcus, a bacterium belonging to the genus Lactobacillus, a bacterium belonging to the genus Turicibacter, a bacterium belonging to the genus Clostridium, a bacterium belonging to the genus Ruminococcus, a bacterium belonging to the genus Veillonella, a bacterium belonging to the genus Bacteroides, a bacterium belonging to the genus Parabacteroides, and a bacterium belonging to the genus Lactococcus. Therefore, in a preferred aspect of the present disclosure, it can be said that the abundance ratio of the intestinal bacterial flora decreases when an abundance ratio (occupancy rate) of the bacterium belonging to the genus Klebsiella, the bacterium belonging to the genus Rothia, or both of them to at least the bacterium belonging to the genus Klebsiella, the bacterium belonging to the genus Rothia, the bacterium belonging to the genus Bifidobacterium, the bacterium belonging to the genus Enterococcus, the bacterium belonging to the genus Streptococcus, the bacterium belonging to the genus Escherichia, the bacterium belonging to the genus Staphylococcus, the bacterium belonging to the genus Lactobacillus, the bacterium belonging to the genus Turicibacter, the bacterium belonging to the genus Clostridium, the bacterium belonging to the genus Ruminococcus, the bacterium belonging to the genus Veillonella, the bacterium belonging to the genus Bacteroides, the bacterium belonging to the genus Parabacteroides, and the bacterium belonging to the genus Lactococcus decreases.

In the present specification, the bacteria whose abundance ratio decreases are bacteria of the genus Klebsiella, bacteria of the genus Rothia, or both of them. In addition, the bacteria whose abundance ratio decreases may be some species or strains belonging to the genus Klebsiella or the genus Rothia.

The decrease in the abundance ratio in the microflora (for example, the non-viral microflora, the germ flora, the bacterial flora, or the like) in the gastrointestinal tract (for example, intestines) of the present disclosure is considered to be due to the action including the inhibitory action on bacterial proliferation and/or the inhibitory action on bacterial colonization in the intestines by the microorganism of the present disclosure.

In the present disclosure, preferably, the “germ flora improvement” further includes increasing an abundance ratio of the bacterium belonging to the genus Bifidobacterium in the intestinal bacterial flora by the microorganism of the present disclosure.

In a preferred embodiment of the present disclosure, it is considered that an abundance ratio of the bacteria increases in the microflora (for example, the non-viral microflora, the germ flora, the bacterial flora, or the like) in the gastrointestinal tract (for example, intestines) when an abundance ratio (occupancy rate) of the bacterium belonging to the genus Bifidobacterium to the bacteria belonging to at least the bacterium belonging to the genus Klebsiella, the bacterium belonging to the genus Rothia, the bacterium belonging to the genus Bifidobacterium, the bacterium belonging to the genus Enterococcus, the bacterium belonging to the genus Streptococcus, the bacterium belonging to the genus Escherichia, the bacterium belonging to the genus Staphylococcus, the bacterium belonging to the genus Lactobacillus, the bacterium belonging to the genus Turicibacter, the bacterium belonging to the genus Clostridium, the bacterium belonging to the genus Ruminococcus, the bacterium belonging to the genus Veillonella, the bacterium belonging to the genus Bacteroides, the bacterium belonging to the genus Parabacteroides, and the bacterium belonging to the genus Lactococcus increases.

Here, the bacteria whose abundance ratio increases are the bacteria belonging to the genus Bifidobacterium, and may be some species or strains belonging to the genus Bifidobacterium.

Examples of the bacterium belonging to the genus Bifidobacterium include Bifidobacterium breve, Bifidobacterium longum, Bifidobacterium adolescentis, Bifidobacterium bifidum, Bifidobacterium catenulatum, and Bifidobacterium pseudocatenulatum. In a preferred aspect, an abundance ratio of not only Bifidobacterium breve contained in the composition of the present disclosure, but also other bacteria belonging to the genus Bifidobacterium may increase.

### (Environment and SDGs)

In another aspect, the present disclosure provides production of a circulation type “product (meat or the like)” that realizes sustainable development goals (SDGs) and is environmentally friendly.

In one aspect, there is provided a method for producing a useful product for a human derived from a useful animal, the method including: a step i) of providing an exogenous microorganism having an ability to convert a component of a photosynthetic organism, such as a plant, that is not a nutrient source in the useful animal into a nutrient source in the useful animal, or a part thereof; a step ii) of introducing the exogenous microorganism or a part thereof into the useful animal; a step iii) of placing the useful animal under conditions under which the useful animal grows; and a step iv) of optionally obtaining the useful product from the useful animal. Here, for the steps i) to ii), any method described elsewhere in the present specification can be used.

In one embodiment, the step ii) may include a step of introducing the exogenous microorganism or a part thereof into the useful animal at least at a part of a time during breeding of the useful animal. In one embodiment, the step ii) may include a step of introducing the exogenous microorganism or a part thereof into the useful animal at least at a part of a time during breeding of the useful animal, and breeding the useful animal without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks.

In one embodiment, the step ii) may include a step of introducing the exogenous microorganism or a part thereof into the useful animal at least at a part of a time during growing of the useful animal. In one embodiment, the step ii) may include a step of introducing an exogenous microorganism or a part thereof into the useful animal at least at a part of a time during growing of the useful animal, and breeding the useful animal without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks. In some embodiments, the growth time can be any period of time during which the body length increases, and in some embodiments, the growth time can be any period of time after beginning of postpartum feed or water ingestion, and can be 1 day, 2 days, 3 days, 4 days, 5 days, 6 days, 7 days, 8 days, 9 days, 10 days, 2 weeks, 3 weeks, or 4 weeks after beginning of postpartum feed or water ingestion. The exogenous microorganism or a part thereof is introduced into the useful animal during the time, such that the exogenous microorganism or a part thereof can be stably maintained in the bacterial flora in the body of the useful animal.

In one embodiment, the method of the present invention may further include a step of optionally confirming that a desired improvement is achieved in the useful animal. In a certain embodiment, the desired improvement may be a change (increase or decrease) in body weight, a change in intestinal bacterial flora, a change (increase or decrease) in fat composition in the body, a change (increase or decrease) in fat percentage in body weight, an increase in intake, or an improvement in digestion and absorption of plant feed.

In one embodiment, the method of the present invention may further include a step i′) of selecting an exogenous microorganism suitable (“compatible”) for the useful animal, or a part thereof, and a step ii′) of introducing the suitable exogenous microorganism or a part thereof into the useful animal. In a certain embodiment, the exogenous microorganism or a part thereof suitable for the useful animal may be an exogenous microorganism or a part thereof that results in a desired improvement or is susceptible to engraftment in the useful animal. In some embodiments, when the useful animal is an individual living in seawater, the exogenous microorganism or a part thereof suitable for the useful animal may be an exogenous microorganism derived from an organism living in seawater or a part thereof. In some embodiments, when the useful animal is an individual living in freshwater, the exogenous microorganism or a part thereof suitable for the useful animal may be an exogenous microorganism derived from an organism living in freshwater or a part thereof. In some embodiments, when the useful animal is an individual living in brackish water, the exogenous microorganism or a part thereof suitable for the useful animal may be an exogenous microorganism derived from an organism living in brackish water or a part thereof.

In the present specification, the step of placing the useful animal under the conditions under which the useful animal grows can be achieved by placing the useful animal in an arbitrary breeding environment. By being placed under such conditions, the introduced exogenous microorganisms or a portion thereof is converted into a nutrient source in the useful animal, such that the useful animal is allowed to obtain more nutrient sources.

Here, the useful organism refers to an animal that produces a useful product for humans (for example, meat, milk, skin, or the like).

In the present specification, the step of obtaining the useful product from the useful animal can be appropriately performed according to the useful product.

In one embodiment, the present disclosure provides a product (meat, fish meat, fish egg, wool, or the like) obtained “directly” from the useful animal as the useful product.

In another embodiment, the useful product includes a product obtained “indirectly” from the useful animal (a processed product such as a can, a hamburger, clothes, or the like).

In one specific embodiment, the present disclosure provides a novel use of a gastrointestinal microflora-derived microorganism focusing on the use of the useful animal. Here, the present disclosure provides a composition for use in a method for producing a useful product for a human derived from a useful animal, the composition containing a microorganism derived from a gastrointestinal microflora or a part thereof, in which the method includes: a step i) of providing an exogenous microorganism having an ability to convert a component of a photosynthetic organism, such as a plant, that is not a nutrient source in the useful animal into a nutrient source in the useful animal, or a part thereof, a step ii) of introducing the exogenous microorganism or a part thereof into the useful animal; a step iii) of placing the useful animal under conditions under which the useful animal grows; and a step iv) of optionally collecting the useful product from the useful animal, and the exogenous microorganism is a microorganism derived from the gastrointestinal microflora.

In one embodiment, the step ii) may include a step of introducing the exogenous microorganism or a part thereof into the useful animal at least at a part of a time during breeding of the useful animal. In one embodiment, the step ii) may include a step of introducing the exogenous microorganism or a part thereof into the useful animal at least at a part of a time during breeding of the useful animal, and breeding the useful animal without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks.

In one embodiment, the step ii) may include a step of introducing the exogenous microorganism or a part thereof into the useful animal at least at a part of a time during growing of the useful animal. In one embodiment, the step ii) may include a step of introducing an exogenous microorganism or a part thereof into the useful animal at least at a part of a time during growing of the useful animal, and breeding the useful animal without administering the microorganism during the rest of the time. In a certain embodiment, the time for the exogenous microorganism or a part thereof may be 1 day, 2 days, 3 days, 4 days, 5 days, 6 days, 7 days, 8 days, 9 days, 10 days, 2 weeks, 3 weeks, or 4 weeks. In some embodiments, the growth time can be any period of time during which the body length increases, and in some embodiments, the growth time can be any period of time after beginning of postpartum feed or water ingestion, and can be 1 day, 2 days, 3 days, 4 days, 5 days, 6 days, 7 days, 8 days, 9 days, 10 days, 2 weeks, 3 weeks, or 4 weeks after beginning of postpartum feed or water ingestion. The exogenous microorganism or a part thereof is introduced into the useful animal during the time, such that the exogenous microorganism or a part thereof can be stably maintained in the bacterial flora in the body of the useful animal.

In one embodiment, the method of the present invention may further include a step of optionally confirming that a desired improvement is achieved in the useful animal. In a certain embodiment, the desired improvement may be a change (increase or decrease) in body weight, a change in intestinal bacterial flora, a change (increase or decrease) in fat composition in the body, a change (increase or decrease) in fat percentage in body weight, an increase in intake, or an improvement in digestion and absorption of plant feed.

In one embodiment, the method of the present invention may further include a step i′) of selecting an exogenous microorganism suitable (“compatible”) for the useful animal, or a part thereof; and a step ii′) of introducing the suitable exogenous microorganism or a part thereof into the useful animal. In a certain embodiment, the exogenous microorganism or a part thereof suitable for the useful animal may be an exogenous microorganism or a part thereof that results in a desired improvement or is susceptible to engraftment in the useful animal. In some embodiments, when the useful animal is an individual living in seawater, the exogenous microorganism or a part thereof suitable for the useful animal may be an exogenous microorganism derived from an organism living in seawater or a part thereof. In some embodiments, when the useful animal is an individual living in freshwater, the exogenous microorganism or a part thereof suitable for the useful animal may be an exogenous microorganism derived from an organism living in freshwater or a part thereof. In some embodiments, when the useful animal is an individual living in brackish water, the exogenous microorganism or a part thereof suitable for the useful animal may be an exogenous microorganism derived from an organism living in brackish water or a part thereof.

In one aspect, the utilization of the present disclosure can provide an environmentally friendly technique, or can contribute to the achievement of sustainable development goals (SDGs) and targets.

The technique of the present disclosure constructs a physical distribution network including not only an agricultural product but also a residue, optimizes (reduction of greenhouse gas (for example, CO₂) emissions and reduction of distribution cost) the entire food chain, optimizes loss in the production, distribution, and consumption processes of food and drink, and easily realizes SDGs.

The utilization of the present disclosure can contribute to achieving sustainable development goals (SDGs) and targets. Sustainable development goals (SDGs) and targets include the following (from “Transforming our world: the 2030 Agenda for Sustainable Development” (provisional translation by Ministry of Foreign Affairs of Japan)).

Goal 1. End poverty in all its forms everywhere

With the technique of the present disclosure, optimal distribution of resources can be achieved, thereby achieving this goal.

Goal 2. End hunger, achieve food security and improved nutrition and promote sustainable agriculture

With the technique of the present disclosure, optimal distribution of resource materials and resources can be achieved, thereby achieving this goal.

Goal 3. Ensure healthy lives and promote well-being for all at all ages

With the technique of the present disclosure, optimal distribution of resources can be achieved taking into account health information, thereby achieving this goal.

Goal 4. Ensure inclusive and equitable quality education and promote lifelong learning opportunities for all

With the technique of the present disclosure, in the process of optimal distribution of resources, an appropriate learning environment can be provided, thereby achieving this goal.

Goal 5. Achieve gender equality and empower all women and girls

With the technique of the present disclosure, optimal distribution of resources can be achieved, and time and other resources for overburdened women and girls empowerment can be ensured, thereby achieving this goal.

Goal 6. Ensure availability and sustainable management of water and sanitation for all

With the technique of the present disclosure, optimal distribution of resources can be achieved while maintaining an adequate sanitation environment, thereby achieving this goal.

Goal 7. Ensure access to affordable, reliable, sustainable and modern energy for all

With the technique of the present disclosure, energy distribution can be appropriately performed by achieving optimal distribution of resources, thereby achieving this goal.

Goal 8. Promote sustained, inclusive and sustainable economic growth, full and productive employment and decent work for all

With the technique of the present disclosure, optimal distribution of resources can be achieved, and foundations for proper economic growth can be fortified, thereby achieving this goal.

Goal 9. Build resilient infrastructure, promote inclusive and sustainable industrialization and foster innovation

With the technique of the present disclosure, the industrial structure can be reformed by achieving optimal distribution of resources, thereby achieving this goal.

Goal 10. Reduce inequality within and among countries

With the technique of the present disclosure, optimal distribution of resources can be achieved, and excessive exploitation of resource raw materials can be leveled, thereby achieving this goal.

Goal 11. Make cities and human settlements inclusive, safe, resilient and sustainable

With the technique of the present disclosure, optimal distribution of resources can be achieved, and the city environment can provide a dramatic improvement of the living conditions, thereby achieving this goal.

Goal 12. Ensure sustainable consumption and production patterns

With the technique of the present disclosure, optimal distribution of resources can be achieved, and sustainable production can be achieved by loss minimization, thereby achieving this goal.

Goal 13. Take urgent action to combat climate change and its impacts

With the technique of the present disclosure, optimal distribution of resources can be achieved, the influence of climate change can be minimized, and at the same time, the greenhouse gas emissions that can be reduced with optimization can be reduced, thereby achieving this goal.

Goal 14. Conserve and sustainably use the oceans, seas and marine resources for sustainable development

With the technique of the present disclosure, optimal distribution of resources can be achieved, and marine resources can be preserved, thereby achieving this goal.

Goal 15. Protect, restore and promote sustainable use of terrestrial ecosystems, sustainably manage forests, combat desertification, and halt and reverse land degradation and halt biodiversity loss

With the technique of the present disclosure, optimal distribution of resources can be achieved, excessive terrestrial ecosystems can be conserved, and biodiversity loss can be minimized, thereby achieving this goal.

Goal 16. Promote peaceful and inclusive societies for sustainable development, provide access to justice for all and build effective, accountable and inclusive institutions at all levels

With the technique of the present disclosure, optimal distribution of resources can be achieved, and a healthy social life can be achieved so that awareness spreads and barriers to accessing justice are lowered, thereby achieving this goal.

Goal 17. Strengthen the means of implementation and revitalize the global partnership for sustainable development

With the technique of the present disclosure, optimal distribution of resources can be achieved, and an environment that is conducive to global partnerships can be provided, thereby achieving this goal.

While the present disclosure targets all of these goals, in one example, the utilization of the present disclosure can contribute to achieving Goals 1 to 3, 7, 12, and 15.

Alternatively, it is possible to cope with allergen-free foods, halal foods for religious reasons, and emotionally-avoidable foods such as vegan, and it is also possible to produce functional foods, highly therapeutic and convalescent hospital foods, and the like.

In the present specification, “or” is used when “at least one or more” of the items listed in the text can be employed. The same applies to “or”. When explicitly described in the present specification as “within the range” of “two values”, the range also includes the two values themselves.

Reference literatures such as scientific literatures, patents, and patent applications cited in the present specification are incorporated herein by reference to the same extent that the entirety of each document is specifically described.

The present disclosure has been described above with reference to preferred embodiments for easy understanding. Hereinafter, the present disclosure will be described based on examples, but the above description and the following examples are provided for the purpose of illustration only and not for the purpose of limiting the present disclosure. Accordingly, the scope of the present disclosure is not limited to the embodiments or examples specifically described in the present specification, but is limited only by the claims.

### EXAMPLES

### Example 1: Isolation and Identification of Significantly Mass-Produced Polyunsaturated Fatty Acid Such as EPA from Gastrointestinal Tract

In the present example, first, bacterial candidates were obtained from the gastrointestinal tract of isaza, which is gobies fish endemic to Lake Biwa.

The isaza intestine contents were suspended in a phosphate buffer solution, seeded on Luria broth (LB) agar medium, and statically cultured at room temperature for several days. The resulting colonies were randomly isolated as isolated strains.

About 1,500 base pairs, which correspond to almost the entire length of 16S rRNA gene, were amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side was determined, and species related to the DNA fragment were searched for by homology search (BLAST) against The National Center for Biotechnology Information (NCBI) 16S rRNA sequence database to identify the genus level.

Eicosapentaenoic acid (EPA) production for each strain isolated was tested as follows.

The isolated strain was cultured in LB medium (10 g tryptone/5 g yeast extract/10 g NaCl/1 L) at 4° C. for about 24 hours and at 18° C. for about 12 hours. As a result, a strain producing a large amount of EPA in both low temperature culture (4° C.) and high temperature culture (18° C.) was identified as Schewanella sp. GI35 strain. For comparison, the literature values (Kawamoto et al, (2009) Journal of Bacteriology 191, 632-640) of the Schewanella livingstonesis Ac10 strain, which is a highly EPA producing bacterium isolated from Antarctic seawater, are also shown.
GI35 strain: 4.2% (18° C.); 12.3% (4° C.)
Ac10 strain: 0.7% (18° C.); 5.1% (4° C.)

### Example 2: Analysis of Production Mode of Polyunsaturated Fatty Acid

In the present example, in order to examine the production mode of the polyunsaturated fatty acid of the GI35 strain, the composition of the free fatty acid contained in the GI35 strain was examined. The GI35 strain was inoculated into LurriaBroth (LB) medium (NaCl 10 g, Bactotryptone 10 g, Yeast extract 5 g, ultrapure water 1,000 ml) with 1/100 volume of a culture solution of the GI35 strain, and shaking culture was performed at 4° C. or 25° C. until reaching an intermediate to late logarithmic proliferation (until an OD was about 1). Thereafter, bacteria were collected, an amount of bacteria having an OD600 of about 15 was dispensed into an Eppendorf tube, and bacteria pellets were prepared by centrifugation and frozen at −80° C. 800 μL of water was added to the bacterial pellets on ice to suspend, and the total lipids were extracted in the same manner as in the common protocol and separated by TLC. In the table, the fatty acid represents the number of carbon atoms and the degree of unsaturation (the number and position of double bonds, and * represents data obtained by reanalyzing (demonstration analysis) undetected fatty acids. GI35-4A represents a value at 4° C., and GI35-25A represents a value at 25° C. The unit, including the lower limit of quantification, is ng.

TABLE 1

| | | | | Lower limit of |
|---|---|---|---|---|
| No. | Fatty acid | GI35-4A | GI35-25A | quantification |
| 1 | C8:0 | N.D. | N.D. | 2000 |
| 2 | C10:0 | N.D. | N.D. | 500 |
| 3 | C12:0 | N.D. | N.D. | 200 |
| 4 | C14:0 | 2100 | 700 | 100 |
| 5 | C14:1n-5 | 800 | N.D. | 200 |
| 6 | C16:0 | 3500 | 3700 | 100 |
| 7 | C16:1n-7 | 33000 | 12000 | 200 |
| 8 | C18:0 | 200 | 100 | 100 |
| 9 | C18:1n-9 | 400 | 1100 | 100 |
| 10 | C18:2n-6* | 18 | 10 | 2 |
| 11 | C18:3n-3* | 12 | N.D. | 2 |
| 12 | C18:3n-6* | 110 | 3.8 | 2 |
| 13 | C18:4n-3 | 1300 | N.D. | 200 |
| 14 | C20:1n-9 | N.D. | N.D. | 100 |
| 15 | C20:2n-6 | N.D. | N.D. | 100 |
| 16 | C20:3n-3 | N.D. | N.D. | 200 |
| 17 | C20:3n-6* | 3.2 | N.D. | 2 |
| 18 | C20:3n-9 | N.D. | N.D. | 100 |
| 19 | C20:4n-3* | 210 | 16 | 10 |
| 20 | C20:4n-6* | 140 | 35 | 10 |
| 21 | C20:5n-3 | 42000 | 2600 | 200 |
| 22 | C22:0 | N.D. | N.D. | 500 |
| 23 | C22:2n-6 | N.D. | N.D. | 200 |
| 24 | C22:4n-6 | N.D. | N.D. | 100 |
| 25 | C22:5n-3* | 200 | 2.6 | 2 |
| 26 | C22:5n-6* | N.D. | N.D. | 2 |
| 27 | C22:6n-3* | 6.9 | 3.9 | 2 |
| 28 | C24:0 | N.D. | N.D. | 500 |
| 29 | C24:5n-3 | N.D. | N.D. | 100 |
| 30 | C24:6n-3 | N.D. | N.D. | 100 |

As a result, the presence of myristic acid, myristoleic acid, palmitic acid, palmitoleic acid, stearic acid, oleic acid, linoleic acid, γ-linolenic acid, α-linolenic acid, stearidonic acid, dihomo-γ-linolenic acid, arachidonic acid, eicosatetraenoic acid (ETA), eicosapentaenoic acid (EPA), docosapentaenoic acid (DPA), and docosahexaenoic acid (DHA) was confirmed.

From these results, the GI35 strain was suggested to contain a fatty acid desaturase that catalyzes the reaction of linoleic acid to γ-linolenic acid (FADS2), a fatty acid elongase that catalyzes the reaction of γ-linolenic acid to dihomo-γ-linolenic acid (EVOL5), a fatty acid desaturase that catalyzes the reaction of dihomo-γ-linolenic acid to arachidonic acid (FADS1), a fatty acid desaturase that catalyzes the reaction of α-linolenic acid to stearidonic acid (FADS2), a fatty acid elongase that catalyzes the reaction of stearidonic acid to eicosatetraenoic acid (ETA) (EVOL5), a fatty acid desaturase that catalyzes the reaction of eicosatetraenoic acid (ETA) to eicosapentaenoic acid (EPA) (FADS1), a fatty acid elongase that catalyzes the reaction from eicosapentaenoic acid (EPA) to docosapentaenoic acid (DPA) (n-3), a fatty acid desaturase that catalyzes the reaction from docosapentaenoic acid (DPA) (n-3) to docosahexaenoic acid (DHA) (n-3), a fatty acid elongase that catalyzes the reaction from arachidonic acid to adrenic acid (n-6), and a fatty acid desaturase that catalyzes the reaction from adrenic acid (n-6) to osbond acid.

### Example 3: Polyunsaturated Fatty Acid Synthase Derived from GI35 Strain

The GI35 strain is cultured and recovered. The recovered strain is suspended in a phosphate buffer and disrupted by French press. The disrupted liquid is centrifuged and the supernatant is dialyzed against a phosphate buffer overnight to obtain a soluble fraction. Arachidonic acid, eicosatetraenoic acid (ETA), eicosapentaenoic acid (EPA), osbond acid, docosapentaenoic acid (DPA), and docosahexaenoic acid (DHA) can be synthesized by mixing materials such as linoleic acid with the GI35 strain or the soluble fraction and incubating the mixture at 18° C. or lower.

The bacterial EPA production system is performed by a polyunsaturated fatty acid synthase (PUFA synthase) similar to a polyketide synthase by an anaerobic biosynthetic pathway, and only EPA is biosynthesized. On the other hand, it is considered that bacteria do not have various biosynthetic routes of polyunsaturated fatty acids by fatty acid desaturases as in mammals, and the polyunsaturated fatty acids as described above are not produced, but it can be said that bacteria exhibit properties different from those of normal bacteria.

### Example 4: Metagenomic Analysis of Intestinal Bacterial Flora of Rainbow Trout Fry to which GI35 Strain is Administered

### Feeding Conditions

Normal feed group: Breeding was performed with feed for rainbow trout (Trout feed super A, Marubeni Nisshin Feed Co., Ltd.) (normal feed: containing about 40% of fish meal) for 6 months.

GI35→normal feed group: Breeding was performed with normal feed to which the GI35 strain (about 3×10⁹ cells/g feed) (GI35 added feed) was added for 1 month, and thereafter, breeding was performed with normal feed for 5 months.

### Metagenomic Analysis of Intestinal Bacterial Flora

For the fry of the normal feed group and the fry of the GI35→normal feed group, the change in the intestinal bacterial flora due to GI35 ingestion and the predicted resulting change in metabolic activity were identified by metagenomic analysis.

### Library Creation and Sequencing

1. Three individuals were randomly collected from each group, and DNA was extracted and purified from the intestinal contents using QIAamp DNA Microbiome Kit.
2. Quantitative measurement of DNA solution: A concentration of the DNA solution was measured using Synergy LX (Biotek) and Quanti Flour dsDNA System (Promega).
3. Library creation: A 16S rDNA library was created using 2-steptailed PCR method.
4. Quantification of library: A concentration of the created library was measured using Synergy H1 (Biotek) and Quanti Flour dsDNA System.
5. Quality confirmation of library: Quality confirmation of the created library was performed using Fragment Analyzer dsDNA 915 Reagent Kit (Advanced Analytical Technologies).
6. Sequencing analysis: Sequencing was performed under the condition of 2×300 bp using MiSeq system and MiSeq Reagent Kit v3 (Illumina).

### Metagenomic Analysis

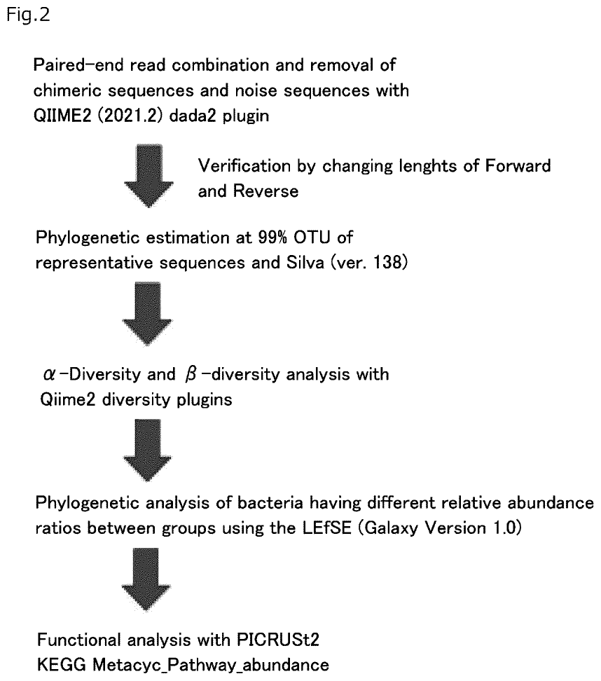

The metagenomic analysis was performed according to the following (FIG. 2).

a. Analysis by Qiime2

Paired-end read combination and removal of chimeric sequences and noise sequences were performed with QIIMffE2 (2021.2) dada2 plugin from FastaQ format files. For the paired-end combination, a length at which the median of the Q-value was 20 or less was used.

b. Diversity Analysis

α- and β-diversity analysis with Qiime2 diversity plugins was performed.

c. Phylogenetic Analysis and Analysis by LEfSE and PICRUSt2

Phylogenetic estimation at 99% OTU of representative sequences and Silva (ver. 138) was performed. Phylogenetic analysis of bacteria having different relative abundance ratios between groups was performed using the LEfSE (Galaxy Version 1.0). Furthermore, comparative analysis of the prediction of the expression gene function in KEGG KEGG Orthology was performed with PICRUSt2.

### (Results)

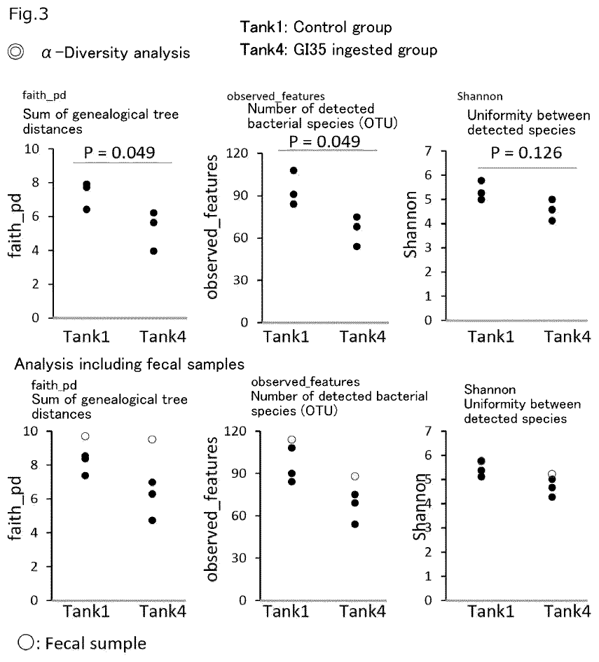

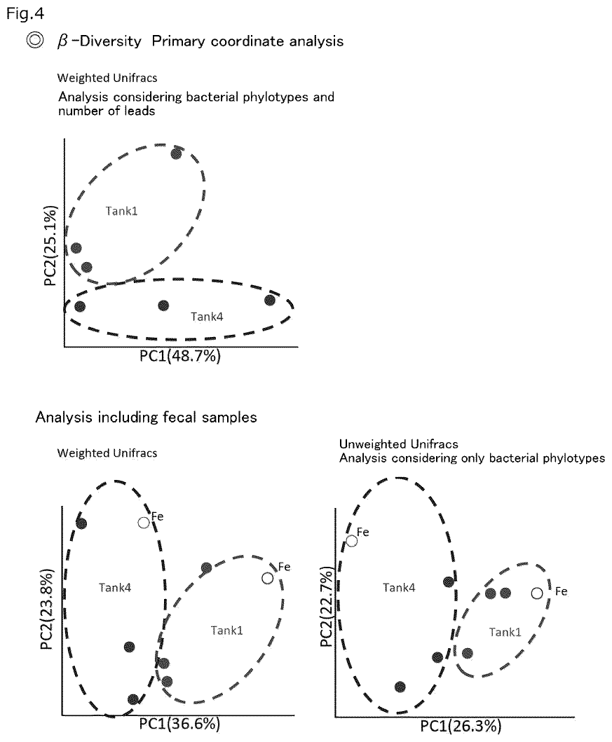

As a result, it could be seen that the diversity of the intestinal bacterial flora of rainbow trout fry fed with the GI35 strain was changed (FIGS. 3 and 4).

### Example 5: Identification of Strain from Japanese Rice Fish Intestinal Tract

Taxon analysis of Japanese rice fish-derived bacteria OR series

The Japanese rice fish intestine contents were suspended in a phosphate buffer solution, seeded on Luria broth (LB) agar medium, and statically cultured at room temperature for several days. The resulting colonies were randomly isolated as isolated strains.

About 1,500 base pairs, which correspond to almost the entire length of 16S rRNA gene, were amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side was determined, and species related to the DNA fragment were searched for by homology search (BLAST) against The National Center for Biotechnology Information (NCBI) 16S rRNA sequence database to identify the genus level. As a result, it was identified as Pseudomonas fluorescens, Pseudomonas extremorientalis, Microbacterium oxydans, Aeromonas veronii, Diaminobutyricmonas aerilata, Bosea robinae, Shinella curvata, Fungi, Pseudomons koreensis, and Aeromonas media.

The sequences of 16S rRNAs of the isolated strains OR1 to OR8 and OR12 to OR14 are as shown in SEQ ID NOs: 4 to 14.

### Example 5A: Identification of Strain from Isaza Intestinal Tract

The isaza intestine contents were suspended in a phosphate buffer solution, seeded on Luria broth (LB) agar medium, and statically cultured at room temperature for several days. The resulting colonies were randomly isolated as isolated strains.

About 1,500 base pairs, which correspond to almost the entire length of 16S rRNA gene, were amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side was determined, and species related to the DNA fragment were searched for by homology search (BLAST) against The National Center for Biotechnology Information (NCBI) 16S rRNA sequence database to identify the genus level. As a result, it was identified as follows.

The sequences of 16S rRNAs of the isolated strains G112, G1431, G171, and GI83 are as shown in SEQ ID NOs: 15 to 18.

TABLE 2

| isolate ID | Genus | Source | Related species |
|---|---|---|---|
| GI2 | Bacillus | Isaza | Bacillus marisflavi |
| GI431 | Aeromonas | Isaza | Aeromonas veronii |
| GI71 | Psychrobacter | Isaza | Psychrobacter faecalis |
| GI83 | Psychrobacter | Isaza | Psychrobacter alimentarius |

### Example 5B: Identification of Microorganism in Japanese Rice Fish Intestinal Tract by Metagenomic Analysis

The Japanese rice fish intestine contents were suspended in a phosphate buffer solution, and library creation and sequencing were performed as follows.
1. Three individuals were randomly collected from each group, and DNA was extracted and purified from the intestinal contents using QIAamp DNA Microbiome Kit.
2. Quantitative measurement of DNA solution: A concentration of the DNA solution was measured using Synergy LX (Biotek) and Quanti Flour dsDNA System (Promega).
3. Library creation: A 16s rDNA library was created using 2-steptailed PCR method.
4. Quantification of library: A concentration of the created library was measured using Synergy H1 (Biotek) and Quanti Flour dsDNA System.
5. Quality confirmation of library: Quality confirmation of the created library was performed using Fragment Analyzer dsDNA 915 Reagent Kit (Advanced Analytical Technologies).
6. Sequencing analysis: Sequencing was performed under the condition of 2×300 bp using MiSeq system and MiSeq Reagent Kit v3 (Illumina). Using Qiime2 (ver. 2020.8), the obtained representative sequence was compared with the 16S rRNA sequence database Greengene (ver. 138) to identify the genus level of the bacterial species constituting the bacterial flora. As a result, it was identified as follows.

TABLE 3-1

| | | |
|---|---|---|
| Acidaminococeus | Japanese rice fish | metagenome |
| Adlercreutzia | Japanese rice fish | metagenome |
| Akkermansia | Japanese rice fish | metagenome |
| Alistipes | Japanese rice fish | metagenome |
| Alloscardovia | Japanese rice fish | metagenome |
| Anaerococcus | Japanese rice fish | metagenome |
| Anaerostipes | Japanese rice fish | metagenome |
| Anaerotruneus | Japanese rice fish | metagenome |
| Bacillus | Japanese rice fish | metagenome |
| Bacteroides | Japanese rice fish | metagenome |
| Bifidobacterium | Japanese rice fish | metagenome |
| Bilophila | Japanese rice fish | metagenome |
| Blautia | Japanese rice fish | metagenome |
| Brachyspira | Japanese rice fish | metagenome |
| Butyricicoccus | Japanese rice fish | metagenome |
| Butyricimonas | Japanese rice fish | metagenome |
| Campylobacter | Japanese rice fish | metagenome |
| Catenibacterium | Japanese rice fish | metagenome |
| Christensenella | Japanese rice fish | metagenome |
| Citrobacter | Japanese rice fish | metagenome |
| Clostridium | Japanese rice fish | metagenome |
| Collinsella | Japanese rice fish | metagenome |
| Coprobacillus | Japanese rice fish | metagenome |
| Coprococcus | Japanese rice fish | metagenome |
| Dehalobacterium | Japanese rice fish | metagenome |
| Desulfovibrio | Japanese rice fish | metagenome |
| Dialister | Japanese rice fish | metagenome |
| Dorea | Japanese rice fish | metagenome |
| Eggerthella | Japanese rice fish | metagenome |
| Enterococcus | Japanese rice fish | metagenome |
| Escherichia | Japanese rice fish | metagenome |
| Faecalibacterium | Japanese rice fish | metagenome |

TABLE 3-2

| | | |
|---|---|---|
| Finegoldia | Japanese rice fish | metagenome |
| Fusobacterium | Japanese rice fish | metagenome |
| Granulicatella | Japanese rice fish | metagenome |
| Haemophilus | Japanese rice fish | metagenome |
| Holdemania | Japanese rice fish | metagenome. |
| Klebsiella | Japanese rice fish | metagenome |
| Lachnobacterium | Japanese rice fish | metagenome |
| Lachnospira | Japanese rice fish | metagenome |
| Lactobacillus | Japanese rice fish | metagenome |
| Lactococcus | Japanese rice fish | metagenome |
| Megamonas | Japanese rice fish | metagenome |
| Megasphaera | Japanese rice fish | metagenome |
| Mitsuokella | Japanese rice fish | metagenome |
| Morganella | Japanese rice fish | metagenome |
| Odoribacter | Japanese rice fish | metagenome |
| Oscillospira | Japanese rice fish | metagenome |
| Oxalobacter | Japanese rice fish | metagenome |
| Parabacteroides | Japanese rice fish | metagenome |
| Paraprevotella | Japanese rice fish | metagenome |
| Peptostreptococcus | Japanese rice fish | metagenome |
| Phascolaretobacterium | Japanese rice fish | metagenome |
| Porphyromonas | Japanese rice fish | metagenome |
| Prevotella | Japanese rice fish | metagenome |
| Pseudomonas | Japanese rice fish | metagenome |
| Pseudoramibacter Eubacterium | Japanese rice fish | metagenome |
| Pyramidobacter | Japanese rice fish | metagenome |
| Roseburia | Japanese rice fish | metagenome |
| Ruminococcus | Japanese rice fish | metagenome |
| Serratia | Japanese rice fish | metagenome |
| Slackia | Japanese rice fish | metagenome |
| Streptococcus | Japanese rice fish | metagenome |
| Succinatimonas | Japanese rice fish | metagenome |

TABLE 3-3

| | | |
|---|---|---|
| Sutterella | Japanese rice fish | metagenome |
| Synergistes | Japanese rice fish | metagenome |
| Turicibacter | Japanese rice fish | metagenome |
| Veillonella | Japanese rice fish | metagenome |

### Example 5C: Identification of Microorganism in Young Yellowtail Intestinal Tract by Metagenomic Analysis

The young yellowtail intestine contents were suspended in a phosphate buffer solution, and library creation and sequencing were performed as follows.
1. Three individuals were randomly collected from each group, and DNA was extracted and purified from the intestinal contents using QIAamp DNA Microbiome Kit.
2. Quantitative measurement of DNA solution: A concentration of the DNA solution was measured using Synergy LX (Biotek) and Quanti Flour dsDNA System (Promega).
3. Library creation: A 16s rDNA library was created using 2-steptailed PCR method.
4. Quantification of library: A concentration of the created library was measured using Synergy H1 (Biotek) and Quanti Flour dsDNA System.
5. Quality confirmation of library: Quality confirmation of the created library was performed using Fragment Analyzer dsDNA 915 Reagent Kit (Advanced Analytical Technologies).
6. Sequencing analysis: Sequencing was performed under the condition of 2×300 bp using MiSeq system and MiSeq Reagent Kit v3 (Illumina). Using Qiime2 (ver. 2020.8), the obtained representative sequence was compared with the 16S rRNA sequence database Greengene (ver. 13_8) to identify the genus level of the bacterial species constituting the bacterial flora. As a result, it was identified as follows.

TABLE 4-1

| | | |
|---|---|---|
| Acetobacter | Young yellowtail | metagenome |
| Acidibacter | Young yellowtail | metagenome |
| Acidobacterium | Young yellowtail | metagenome |
| Acidothermus | Young yellowtail | metagenome |
| Actibacter | Young yellowtail | metagenome |
| Allorhizobium-Neorhizobium- | Young yellowtail | metagenome |
| Pararhizobium-Rhizobium | | |
| Anaerococcus | Young yellowtail | metagenome |
| Anaerolinea | Young yellowtail | metagenome |
| Anaeromyxobacter | Young yellowtail | metagenome |
| Aquabacterium | Young yellowtail | metagenome |
| Aquisphaera | Young yellowtail | metagenome |
| Arenimonas | Young yellowtail | metagenome |
| Azovibrio | Young yellowtail | metagenome |
| Bacillus | Young yellowtail | metagenome |
| Bacteroides | Young yellowtail | metagenome |
| Bacteroidetes bacterium | Young yellowtail | metagenome |
| Barrientosiimonas | Young yellowtail | metagenome |
| Bdellovibrio | Young yellowtail | metagenome |
| Bellilinea | Young yellowtail | metagenome |
| Blastocatella | Young yellowtail | metagenome |
| Blastopirellula | Young yellowtail | metagenome |
| Bradyrhizobium | Young yellowtail | metagenome |
| Brevundimonas | Young yellowtail | metagenome |
| Bryobacter | Young yellowtail | metagenome |
| Caldisericum | Young yellowtail | metagenome |
| Candidatus Hepatincola | Young yellowtail | metagenome |
| Candidatus Udaeobacter | Young yellowtail | metagenome |
| Chlorobi bacterium | Young yellowtail | metagenome |
| Chryseobacterium | Young yellowtail | metagenome |
| Chthoniobacter | Young yellowtail | metagenome |
| Citreitalea | Young yellowtail | metagenome |

TABLE 4-2

| | | |
|---|---|---|
| Clostridium | Young yellowtail | metagenome |
| Corynebacterium | Young yellowtail | metagenome |
| Crenothrix | Young yellowtail | metagenome |
| Cutibacterium | Young yellowtail | metagenome |
| Cyanobium | Young yellowtail | metagenome |
| Dermacoccus | Young yellowtail | metagenome |
| Desulfuromonas | Young yellowtail | metagenome |
| Devosia | Young yellowtail | metagenome |
| Dinghuibacter | Young yellowtail | metagenome |
| Enhydrobacter | Young yellowtail | metagenome |
| Enterococcus | Young yellowtail | metagenome |
| Erysipelothrix | Young yellowtail | metagenome |
| Exiguobacterium | Young yellowtail | metagenome |
| Ferruginibacter | Young yellowtail | metagenome |
| Flavobacterium | Young yellowtail | metagenome |
| Fluviicola | Young yellowtail | metagenome |
| Fonticella | Young yellowtail | metagenome |
| Fusobacterium | Young yellowtail | metagenome |
| Gallionella | Young yellowtail | metagenome |
| Geobacter | Young yellowtail | metagenome |
| Geothrix | Young yellowtail | metagenome |
| Halomonas | Young yellowtail | metagenome |
| Hydrogenophaga | Young yellowtail | metagenome |
| Hydrogenophilus | Young yellowtail | metagenome |
| Hyphomicrobium | Young yellowtail | metagenome |
| Ignavibacterium | Young yellowtail | metagenome |
| Immundisolibacter | Young yellowtail | metagenome |
| Kocuria | Young yellowtail | metagenome |
| Lacihabitans | Young yellowtail | metagenome |
| Lactobacillus | Young yellowtail | metagenome |
| Lactococcus | Young yellowtail | metagenome |
| Lawsonella | Young yellowtail | metagenome |

TABLE 4-3

| | | |
|---|---|---|
| Legionella | Young yellowtail | metagenome |
| Leptolinea | Young yellowtail | metagenome |
| Limnobacter | Young yellowtail | metagenome |
| Longilinea | Young yellowtail | metagenome |
| Luteolibacter | Young yellowtail | metagenome |
| Massilia | Young yellowtail | metagenome |
| Methylobacter | Young yellowtail | metagenome |
| Methylocystis | Young yellowtail | metagenome |
| Methylotenera | Young yellowtail | metagenome |
| Microbacterium | Young yellowtail | metagenome |
| Mycobacterium | Young yellowtail | metagenome |
| Novosphingobium | Young yellowtail | metagenome |
| Paludibaculum | Young yellowtail | metagenome |
| Paracoccus | Young yellowtail | metagenome |
| Paraperlucidibaca | Young yellowtail | metagenome |
| Pedomicrobium | Young yellowtail | metagenome |
| Phaeodactylbacter | Young yellowtail | metagenome |
| Phreatobacter | Young yellowtail | metagenome |
| Piscinibacter | Young yellowtail | metagenome |
| Porphyrobacter | Young yellowtail | metagenome |
| Prasinophyceae | Young yellowtail | metagenome |
| Prevotella | Young yellowtail | metagenome |
| Prosthecobacter | Young yellowtail | metagenome |
| Proteiniphilum | Young yellowtail | metagenome |
| Pseudohongiella | Young yellowtail | metagenome |
| Pseudomonas | Young yellowtail | metagenome |
| Pseudonocardia | Young yellowtail | metagenome |
| Pseudorhodobacter | Young yellowtail | metagenome |
| Rhodobacter | Young yellowtail | metagenome |
| Rhodomicrobium | Young yellowtail | metagenome |
| Rickettsiella | Young yellowtail | metagenome |
| Roseiarcus | Young yellowtail | metagenome |

TABLE 4-4

| | | |
|---|---|---|
| Roseomonas | Young yellowtail | metagenome |
| Rubripirellula | Young yellowtail | metagenome |
| Ruminiclostridium | Young yellowtail | metagenome |
| Shewanella | Young yellowtail | metagenome |
| Soehngenia | Young yellowtail | metagenome |
| Solibacter | Young yellowtail | metagenome |
| Sphaerochaeta | Young yellowtail | inetagenome |
| Sphingobacteriales | Young yellowtail | metagenome |
| Sphingomonas | Young yellowtail | metagenome |
| Spirochaeta | Young yellowtail | metagenome |
| Staphylococcus | Young yellowtail | metagenome |
| Sulfuritalea | Young yellowtail | metagenome |
| Synechococcus | Young yellowtail | metagenome |
| Taibaiella | Young yellowtail | metagenome |
| Tenacibaculum | Young yellowtail | metagenome |
| Terrimonas | Young yellowtail | metagenome |
| Thermomonas | Young yellowtail | metagenome |
| Treponema | Young yellowtail | metagenome |
| Trichodesmium | Young yellowtail | metagenome |
| Williamsia | Young yellowtail | metagenome |

### Example 6: Metabolism of Japanese Rice Fish Intestinal Tract Isolated Strain

The cellulolytic activity and ligninolytic activity of the strain isolated in Example 5, the culture supernatant of the strain, and the lysate of the strain were measured by the following methods.

The degradation activity of each of the isolated strain, the culture supernatant of the strain, and the lysate of the strain was measured as follows.

### 1. Measurement of Cellulase Activity Utilizing Proliferation Process of Bacteria

Various strains were seeded on a 1.7% agar medium containing 0.2% sodium nitrate, 0.1% dipotassium hydrogen phosphate, 0.05% magnesium sulfate, 0.05% potassium chloride, 0.2% sodium carboxymethyl cellulose sodium salt, and 0.02% peptone, and allowed to stand at 28° C. for 2 days. A 0.33% iodine solution containing 0.67% potassium iodide was overlaid on the agar medium and then rapidly removed, and an image was captured. The intensity of the cellulase activity was indicated in four levels from +++ to −from the size of pale yellow stained rings around the bacterial mass.

### 2. Measurement of Cellulase Activity Utilizing Bacteria Proliferation Solution

Various strains were cultured in LB medium at 25° C. for 1 to 2 days, and a culture solution, a culture centrifugal supernatant solution, or a surfactant treatment culture solution was used as a test specimen. The cellulase activity was measured using Cellulase Assay Kit (CellG5 Method, Megazyme). The test specimen and the enzyme substrate of the kit were mixed, the mixture was heated at 40° C. for 10 minutes, 2% tris hydrochloric acid (pH 10) was added in a 15-fold amount, and then, the absorbance at 400 nm was measured. From the intensity of absorbance, the intensity of the cellulase activity was expressed in five levels of +++ to ± and −.

### 3. Measurement of Xylanase Activity Utilizing Bacteria Proliferation Solution

Various strains were cultured in LB medium at 25° C. for 1 to 2 days, and a culture solution or a culture centrifugal supernatant solution was used as a test specimen. The xylanase activity was measured using Xylanase Assay Kit (XylX6 Method, Megazyme). The test specimen and the enzyme substrate of the kit were mixed, the mixture was heated at 40° C. for 10 minutes, 2% tris hydrochloric acid (pH 10) was added in a 15-fold amount, and then, the absorbance at 400 nm was measured. From the intensity of absorbance, the intensity of the xylanase activity was expressed in three levels of + to ± and −.

### 4. Measurement of Lignin Degradation Activity Utilizing Proliferation Process of Bacteria

Each strain was cultured in LB medium containing Remazol Brilliant Blue R at 25° C. for 2 to 3 days, and the absorbance of the centrifuged supernatant at 592 nm was measured. From the intensity of the decrease in absorbance, the intensity of the lignin degradation activity was expressed in five levels of +++ to ± and −.

The results are shown in Table 5.

TABLE 5

| | Cellulolysis | | | | | Lignolysis |
|---|---|---|---|---|---|---|
| | Cells | live | lysed | live | lysed | Cells |
| Strain | agar | cells | cells | cells | cells | liquid |
| name | iodine | cellulose | | xylanase | | RBBR |
| OR1 | + | ++ | ± | + | + | ± |
| OR2 | + | ++ | ± | + | + | +++ |
| OR3 | +++ | ++ | + | + | + | +++ |
| OR4 | +++ | ++ | − | + | ± | +++ |
| OR5 | +++ | + | − | − | − | ++ |
| OR6 | +++ | + | − | − | − | ++ |
| OR7 | − | (−) | ++ | (−) | + | − |
| OR8 | +++ | + | − | − | − | ++ |
| OR9 | +++ | (−) | +++ | (−) | − | − |
| OR10 | − | (−) | ± | (−) | − | − |
| OR11 | − | (−) | ++ | (−) | − | ± |
| OR12 | + | ++ | − | + | + | ++ |
| OR13 | +++ | − | ++ | ± | − | ± |
| OR14 | − | ++ | − | + | ± | ± |
| GI35 | − | − | − | − | ± | ± |
| E. Coli | − | − | − | − | − | − |

### Example 6A: Obtaining of Variant Strain of GI35

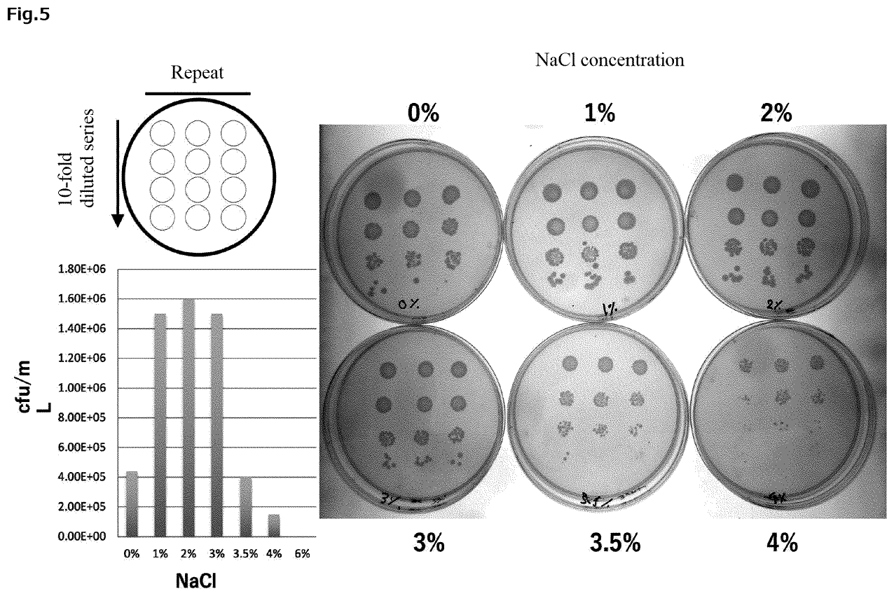

The GI35 strain isolated in Example 1 as the GI35 strain was subjected to streak culture on a 4% NaCl-containing nutrient broth agar medium, and a single colony with excellent growth was selected and subjected to streak culture again on a new 4% NaCl-containing nutrient broth agar medium (FIG. 5).

Not only the salt concentration but also temperature characteristics and the like can be changed (strains having a high-temperature viability and productivity).

By these operations, the GI35 variant strain that survived at a high salt concentration (for example, 4% NaCl) and had desired properties was obtained.

Although no EPA synthesis was measured for substrains that are sorted at a high salt concentration, it is considered that the variant strain probably has the same biosynthetic ability as the parent strain.

### Example 6B: Obtaining of Desired Strain from Sea Bream

The sea bream intestine contents are suspended in a phosphate buffer solution, seeded on Luria broth (LB) agar medium, and statically cultured at room temperature for several days. The resulting colonies are randomly isolated as isolated strains.

About 1,500 base pairs, which correspond to almost the entire length of 16S rRNA gene, are amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side is determined, and species related to the DNA fragment are searched for by homology search (BLAST) against The National Center for Biotechnology Information (NCBI) 16S rRNA sequence database to identify the genus level.

### Example 6C: Obtaining of Desired Strain from Bastard Halibut

The bastard halibut intestine contents are suspended in a phosphate buffer solution, seeded on Luria broth (LB) agar medium, and statically cultured at room temperature for several days. The resulting colonies are randomly isolated as isolated strains.

About 1,500 base pairs, which correspond to almost the entire length of 16S rRNA gene, are amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side is determined, and species related to the DNA fragment are searched for by homology search (BLAST) against The National Center for Biotechnology Information (NCBI) 16S rRNA sequence database to identify the genus level.

### Example 6D: Obtaining of Desired Strain from Other Japanese Rice Fish

The intestine contents of Japanese rice fish capable of growing in a desired growth environment are suspended in a phosphate buffer solution, seeded on Luria broth (LB) agar medium, and statically cultured at room temperature for several days. The resulting colonies are randomly isolated as isolated strains.

About 1,500 base pairs, which correspond to almost the entire length of 16S rRNA gene, are amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side is determined, and species related to the DNA fragment are searched for by homology search (BLAST) against The National Center for Biotechnology Information (NCBI) 16S rRNA sequence database to identify the genus level.

### Example 6E: Obtaining of Desired Strain from Goat

The goat intestine contents are suspended in a phosphate buffer solution, seeded on Luria broth (LB) agar medium, and statically cultured at room temperature for several days. The resulting colonies are randomly isolated as isolated strains.

About 1,500 base pairs, which correspond to almost the entire length of 16S rRNA gene, are amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side is determined, and species related to the DNA fragment are searched for by homology search (BLAST) against The National Center for Biotechnology Information (NCBI) 16S rRNA sequence database to identify the genus level.

### Example 6F: Obtaining of Desired Strain from Meerkat

The meerkat intestine contents are suspended in a phosphate buffer solution, seeded on Luria broth (LB) agar medium, and seeded on at room temperature for several days. The resulting colonies are randomly isolated as isolated strains.

About 1,500 base pairs, which correspond to almost the entire length of 16S rRNA gene, are amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side is determined, and species related to the DNA fragment are searched for by homology search (BLAST) against The National Center for Biotechnology Information (NCBI) 16S rRNA sequence database to identify the genus level.

### Example 7: Production 1 of Rainbow Trout Fry with Modified Microflora in Gastrointestinal Tract

In the same manner as that of Example 5, bacteria are isolated from an intestinal bacterial flora of an individual exhibiting improved features of modification of fatty acid metabolism. Rainbow trout fry are fed normal feed for 1 month and then fed normal feed to which isolated bacteria are added (isolated strain-added feed) for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved ability to metabolize fatty acids compared to rainbow trout fry usually fed only with normal feed.

### Example 8: Production 2 of Rainbow Trout Fry with Modified Microflora in Gastrointestinal Tract

In the same manner as that of Example 5, bacteria are isolated from an intestinal bacterial flora of an individual exhibiting improved features of modification of amino acid metabolism. Rainbow trout fry are fed normal feed for 1 month and then fed normal feed to which isolated bacteria are added (isolated strain-added feed) for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved ability to metabolize amino acids compared to rainbow trout fry usually fed only with normal feed.

### Example 9: Production 3 of Rainbow Trout Fry with Modified Microflora in Gastrointestinal Tract

In the same manner as that of Example 5, bacteria are isolated from an intestinal bacterial flora of an individual exhibiting improved features of modification of cellulose, hemicellulose or lignin degradation. Rainbow trout fry are fed normal feed for 1 month and then fed normal feed to which isolated bacteria are added (isolated strain-added feed) for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have cellulose, hemicellulose or lignin degradation as compared to rainbow trout fry usually fed only with normal feed.

### Example 10: Production 4 of Rainbow Trout Fry with Modified Microflora in Gastrointestinal Tract

In the same manner as that of Example 5, bacteria are isolated from an intestinal bacterial flora of an individual exhibiting improved features of growth promotion. Rainbow trout fry are fed normal feed for 1 month and then fed normal feed to which isolated bacteria are added (isolated strain-added feed) for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved growth rate compared to rainbow trout fry usually fed only with normal feed.

### Example 11: Production 5 of Rainbow Trout Fry with Modified Microflora in Gastrointestinal Tract

In the same manner as that of Example 5, bacteria are isolated from an intestinal bacterial flora of an individual exhibiting improved features of essential nutrient (essential fatty acids, essential amino acids, vitamins, and the like) contents. Rainbow trout fry are fed normal feed for 1 month and then fed normal feed to which isolated bacteria are added (isolated strain-added feed) for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved nutrient (essential fatty acids, essential amino acids, vitamins, and the like) content compared to rainbow trout fry usually fed only with normal feed.

### Example 12: Production 6 of Rainbow Trout Fry with Modified Microflora in Gastrointestinal Tract

In the same manner as that of Example 5, bacteria are isolated from an intestinal bacterial flora of an individual exhibiting improved features of enhancement of immune response ability. Rainbow trout fry are fed normal feed for 1 month and then fed normal feed to which isolated bacteria are added (isolated strain-added feed) for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have enhancement of immune response ability compared to rainbow trout fry usually fed only with normal feed.

### Example 12A: Production of Sea Bream Fry with Modified Microflora in Gastrointestinal Tract

In the same manner as that of Example 5, bacteria are isolated from an intestinal bacterial flora of an individual exhibiting improved features of growth promotion. Sea bream fry are fed normal feed for 1 month and then fed normal feed to which isolated bacteria are added (isolated strain-added feed) for 5 months to produce sea bream fry in which the bacterial flora in the gastrointestinal tract is modified. As a control group, sea bream fry fed only with normal feed for 6 months are also produced.

It can be seen that sea bream fry fed with the isolated bacteria have an improved growth rate compared to sea bream fry usually fed only with normal feed.

### Example 12B: Production of Tuna Fry with Modified Microflora in Gastrointestinal Tract

In the same manner as that of Example 5, bacteria are isolated from an intestinal bacterial flora of an individual exhibiting improved features of growth promotion. Tuna fry are fed normal feed for 1 month and then fed normal feed to which isolated bacteria are added (isolated strain-added feed) for 5 months to produce tuna fry in which the bacterial flora in the gastrointestinal tract is modified. As a control group, tuna fry fed only with normal feed for 6 months are also produced.

It can be seen that tuna fry fed with the isolated bacteria have an improved growth rate compared to tuna fry usually fed only with normal feed.

### Example 12C: Production of Fugu Fry with Modified Microflora in Gastrointestinal Tract

In the same manner as that of Example 5, bacteria are isolated from an intestinal bacterial flora of an individual exhibiting improved features of growth promotion. Fugu fry are fed normal feed for 1 month and then fed normal feed to which isolated bacteria are added (isolated strain-added feed) for 5 months to produce fugu fry in which the bacterial flora in the gastrointestinal tract is modified. As a control group, fugu fry fed only with normal feed for 6 months are also produced.

It can be seen that fugu fry fed with the isolated bacteria have an improved growth rate compared to fugu fry usually fed only with normal feed.

### Example 12D: Production of Yellowtail Fry with Modified Microflora in Gastrointestinal Tract

In the same manner as that of Example 5, bacteria are isolated from an intestinal bacterial flora of an individual exhibiting improved features of growth promotion. Yellowtail fry are fed normal feed for 1 month and then fed normal feed to which isolated bacteria are added (isolated strain-added feed) for 5 months to produce yellowtail fry in which the bacterial flora in the gastrointestinal tract is modified. As a control group, tuna fry fed only with normal feed for 6 months are also produced.

It can be seen that yellowtail fry fed with the isolated bacteria have an improved growth rate compared to yellowtail fry usually fed only with normal feed.

### Example 12E: Production of Carp Fry with Modified Microflora in Gastrointestinal Tract

In the same manner as that of Example 5, bacteria are isolated from an intestinal bacterial flora of an individual exhibiting improved features of growth promotion. Carp fry are fed normal feed for 1 month and then fed normal feed to which isolated bacteria are added (isolated strain-added feed) for 5 months to produce carp fry in which the bacterial flora in the gastrointestinal tract is modified. As a control group, carp fry fed only with normal feed for 6 months are also produced.

It can be seen that carp fry fed with the isolated bacteria have an improved growth rate compared to carp fry usually fed only with normal feed.

### Example 13: Increase in Polyunsaturated Fatty Acid Content of Rotifer by Feed Containing GI35 Strain

### Feeding Conditions

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the rotifers in the GI35→normal feed group have an increased polyunsaturated fatty acid content compared to the rotifers in the normal feed group.

### Example 14: Increase in Polyunsaturated Fatty Acid Content of Shrimp by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the whiteleg shrimps in the GI35→normal feed group have an increased polyunsaturated fatty acid content compared to the whiteleg shrimps in the normal feed group.

### Example 15: Increase in Polyunsaturated Fatty Acid Content of Cockle by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the cockles in the GI35→normal feed group have an increased polyunsaturated fatty acid content compared to the cockles in the normal feed group.

### Example 16: Increase in Polyunsaturated Fatty Acid Content of Octopus by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the octopuses in the GI35→normal feed group have an increased polyunsaturated fatty acid content compared to the octopuses in the normal feed group.

### Example 17: Increase in Polyunsaturated Fatty Acid Content of Frog by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the frogs in the GI35→normal feed group have an increased polyunsaturated fatty acid content compared to the frogs in the normal feed group.

### Example 18: Increase in Polyunsaturated Fatty Acid Content of Softshell Turtle by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the softshell turtles in the GI35→normal feed group have an increased polyunsaturated fatty acid content compared to the softshell turtles in the normal feed group.

### Example 19: Increase in Polyunsaturated Fatty Acid Content of Chicken by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the chickens in the GI35→normal feed group have an increased polyunsaturated fatty acid content compared to the chickens in the normal feed group.

### Example 20: Increase in Polyunsaturated Fatty Acid Content of Mouse by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the mice in the GI35→normal feed group have an increased polyunsaturated fatty acid content compared to the mice in the normal feed group.

### Example 20A: Safety Test of Mouse Fed with Feed Containing GI35 Strain

The GI35 strain is proliferated in LB medium and prepared so that the number of colonies in 0.2 mL of physiological saline is 10⁸, 10⁷, and 10⁶. Mice (Balb/c, C57BL, ICR, and the like) are orally administered with 0.2 mL of the bacterial strain once every 1 to 3 days for 2 weeks, and body weight measurement and life and death observation of the mice are performed once every 1 to 2 days from immediately before administration until 4 weeks after the start of administration. When the body weight decreases to 80% from immediately before the bacterial strain administration, the mouse is determined to die and euthanized. The 50% mouse lethal dose (MLD₅₀) is calculated from the number of dead mice at 4 weeks, and when more than half of the mice survive even at the maximum number of bacteria, it is indicated as “MLD₅₀>10⁸ colony-forming bacteria number”.

As a result, it can be seen that mice receiving 10⁸-colony forming bacteria survive at 4 weeks.

### Example 20B: Intestinal Colonization Test of Mouse Fed with Feed Containing GI35 Strain

The GI35 strain is proliferated in LB medium, and the strain is adjusted to the number of bacteria that does not cause mouse death in 0.2 mL of physiological saline. Mice (Balb/c, C57BL, ICR, and the like) are orally administered 0.2 mL of each strain once every 1 to 3 days for 2 weeks. The feces of the mice are collected as needed during the period of bacterial administration, mouse intestines are collected after 3 weeks, 4 weeks, and 6 weeks from the start of administration, and the presence or absence of administered bacteria in the feces and intestines is examined by 16S ribosome analysis.

As a result, it is found that the GI35 strain engrafted in the intestine at the time points 3 weeks, 4 weeks, and 6 weeks after the start of administration.

### Example 20C: Examination of Use of Plant Fibers as Feed by Mouse Fed with Feed Containing GI35 Strain

Cellulose, bran, crushed corrugated cardboard, and the like are used as dietary fibers, and the GI35 strain proliferated in the LB medium is mixed and heated at various bacterial amounts. Mice (Balb/c, C57BL, ICR, and the like) are bred in a normal feed breeding group, a normal feed and dietary fiber mixed breeding group, and a dietary fiber breeding group, and body weight measurement and life and death observation are performed for two weeks immediately before the start of the test. When the body weight decreases to 80% from immediately before the bacterial strain administration, the mouse is determined to die and euthanized. Compared to the normal feed breeding group, a significant difference specimen of the number of deaths and the rate of weight loss of mice in each group is performed.

As a result, it can be seen that the mice survive in the group fed with any feed. From this, it can be seen that the mice can use the plant fibers as a nutrient source.

### Example 21: Increase in Polyunsaturated Fatty Acid Content of Pig by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the pigs in the GI35→normal feed group have an increased polyunsaturated fatty acid content compared to the pigs in the normal feed group.

### Example 22: Increase in Polyunsaturated Fatty Acid Content of Cow by Feed Containing GI35 Strain

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

It can be seen that the cows in the GI35→normal feed group have an increased polyunsaturated fatty acid content compared to the cows in the normal feed group.

### Example 23: Growth of Pig by Bacterial Flora Derived from Japanese Rice Fish Having Fiber Utilization Ability of Photosynthetic Organism Such as Plant

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of a photosynthetic organism such as a plant is increased in the case of pigs in the fiber utilizing bacteria of the photosynthetic organism such as a plant→normal feed group as compared with the pigs in the normal feed group.

### Example 24: Growth of Cow by Bacterial Flora Derived from Japanese Rice Fish Having Fiber Utilization Ability of Photosynthetic Organism Such as Plant

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of cows in the fiber utilizing bacteria of the photosynthetic organism such as a plant→normal feed group as compared with the cows in the normal feed group.

### Example 25: Growth of Fish (Yellowtail) by Bacterial Flora Derived from Japanese Rice Fish Having Fiber Utilization Ability of Photosynthetic Organism Such as Plant

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of yellowtails in the fiber utilizing bacteria of the photosynthetic organism such as a plant→normal feed group as compared with the yellowtails in the normal feed group.

### Example 26: Growth of Cow by Bacterial Flora Derived from Japanese Rice Fish Having Fiber Utilization Ability of Photosynthetic Organism Such as Plant

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of cows in the fiber utilizing bacteria of the photosynthetic organism such as a plant→normal feed group as compared with the cows in the normal feed group. Increased feed utilization efficiency results in a reduction in feed required for growth, a reduction in non-edible portions that should be discarded, and a reduction in energy associated with food transport.

### Example 27: Example of Modification of Immune Function and Anti-Inflammatory and Anti-Infective Function

Normal feed group: Breeding is performed with normal feed for 6 months.

GI35→normal feed group: Breeding is performed with normal feed to which the GI35 strain is added (GI35 added feed) for 1 month, and thereafter, breeding is performed with normal feed for 5 months.

Blood is collected from the mice in the GI35→normal feed group and the mice in the normal feed group, and the concentration of lipid mediators (for example, metabolites of arachidonic acid, eicosapentaenoic acid, and docosahexaenoic acid) in the blood is measured using a mass spectrometer.

It can be seen that the mice in the GI35→normal feed group have an increased lipid mediator concentration compared to the mice in the normal feed group.

From the above, it can be confirmed that the introduced strain enhances the immune response ability due to enhanced production of polyunsaturated fatty acids and lipid mediators as metabolites thereof in the intestinal tract.

### Example 28: Production of Direct Product (Meat)

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of cows in the fiber utilizing bacteria of the photosynthetic organism such as a plant→normal feed group as compared with the cows in the normal feed group. Increased feed utilization efficiency results in a reduction in feed required for growth, a reduction in non-edible portions that should be discarded, and a reduction in energy associated with food transport, such that meat can be produced at low cost.

### Example 29: Production of Direct Product (Fish)

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of yellowtails in the fiber utilizing bacteria of the photosynthetic organism such as a plant→normal feed group as compared with the yellowtails in the normal feed group. Increased feed utilization efficiency results in a reduction in feed required for growth, a reduction in non-edible portions that should be discarded, and a reduction in energy associated with food transport, such that fish meat can be produced at low cost.

### Example 30: Production of Direct Product (Milk)

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of cows in the fiber utilizing bacteria of the photosynthetic organism such as a plant→normal feed group as compared with the cows in the normal feed group. Increased feed utilization efficiency results in a reduction in feed required for growth, a reduction in non-edible portions that should be discarded, and a reduction in energy associated with food transport, such that milk can be produced at low cost.

### Example 31: Production of Indirect Product (Processed Product or Meat Product)

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of cows in the fiber utilizing bacteria of the photosynthetic organism such as a plant→normal feed group as compared with the cows in the normal feed group. Increased feed utilization efficiency results in a reduction in feed required for growth, a reduction in non-edible portions that should be discarded, and a reduction in energy associated with food transport, such that a meat processed product such as smoked meat or canned cooked meat can be produced at low cost.

### Example 32: Production of Indirect Product (Processed Product or Dairy Product)

Normal feed group: Breeding is performed with normal feed for 6 months.

Fiber utilizing bacteria of photosynthetic organism such as plant→normal feed group: Breeding is performed with normal feed to which a strain derived from Japanese rice fish having a fiber utilization ability of a photosynthetic organism such as a plant is added (feed containing fiber utilizing bacteria of a photosynthetic organism such as a plant) for 1 month, and thereafter, breeding is performed with normal feed for 5 months.

It can be seen that a fiber utilization amount of photosynthetic organism such as a plant is increased in the case of cows in the fiber utilizing bacteria of the photosynthetic organism such as a plant→normal feed group as compared with the cows in the normal feed group. Increased feed utilization efficiency results in a reduction in feed required for growth, a reduction in non-edible portions that should be discarded, and a reduction in energy associated with food transport, such that a milk-processed product such as cheese or yogurt can be produced at low cost.

### Example 33: Microorganism Product Derived from Intestinal Bacterial Flora

In the same manner as that of Example 5, strains are isolated from an intestinal bacterial flora of an individual that can biosynthesize essential vitamins for humans, and among the isolated strains, a strain that can biosynthesize essential vitamins is screened.

By feeding the strain to a cow, a cow containing a large amount of essential vitamins can be obtained from the cow fed with the strain.

### Example 34: Growth of Pig by Bacterial Flora Derived from Meerkat Having Chitin Utilizing Ability

Normal feed group: Breeding is performed with normal feed for 6 months.

Chitin utilizing bacteria→normal feed group: Breeding is performed with normal feed to which a strain having a chitin utilizing ability derived from a meerkat is added (chitin utilizing bacteria added feed) for 1 month, and thereafter, breeding is performed with normal feed for 5 months.

It can be seen that the meerkats in the chitin utilizing bacteria→normal feed group have an increased chitin utilization amount compared to the meerkats in the normal feed group.

### Example 35: Growth of Cow by Bacterial Flora Derived from Meerkat Having Chitin Utilizing Ability

Normal feed group: Breeding is performed with normal feed for 6 months.

Chitin utilizing bacteria→normal feed group: Breeding is performed with normal feed to which a strain having a chitin utilizing ability derived from a meerkat is added (chitin utilizing bacteria added feed) for 1 month, and thereafter, breeding is performed with normal feed for 5 months.

It can be seen that the cows in the chitin utilizing bacteria→normal feed group have an increased chitin utilization amount compared to the cows in the normal feed group.

### Example 36: Production A1 of Rainbow Trout Fry with Modified Microflora in Gastrointestinal Tract

In the same manner as that of Example 5, bacteria are isolated from an intestinal bacterial flora of an individual exhibiting improved features of modification of fatty acid metabolism. The rainbow trout fry are fed normal feed to which the isolated bacteria are added (isolated strain-added feed) for 1 month and then fed normal feed for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved ability to metabolize fatty acids compared to rainbow trout fry usually fed only with normal feed.

### Example 37: Production A2 of Rainbow Trout Fry with Modified Microflora in Gastrointestinal Tract

In the same manner as that of Example 5, bacteria are isolated from an intestinal bacterial flora of an individual exhibiting improved features of modification of amino acid metabolism. The rainbow trout fry are fed normal feed to which the isolated bacteria are added (isolated strain-added feed) for 1 month and then fed normal feed for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved ability to metabolize amino acids compared to rainbow trout fry usually fed only with normal feed.

### Example 38: Production A3 of Rainbow Trout Fry with Modified Microflora in Gastrointestinal Tract

In the same manner as that of Example 5, bacteria are isolated from an intestinal bacterial flora of an individual exhibiting improved features of modification of cellulose, hemicellulose or lignin degradation. The rainbow trout fry are fed normal feed to which the isolated bacteria are added (isolated strain-added feed) for 1 month and then fed normal feed for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have cellulose, hemicellulose or lignin degradation as compared to rainbow trout fry usually fed only with normal feed.

### Example 39: Production A4 of Rainbow Trout Fry with Modified Microflora in Gastrointestinal Tract

In the same manner as that of Example 5, bacteria are isolated from an intestinal bacterial flora of an individual exhibiting improved features of growth promotion. The rainbow trout fry are fed normal feed to which the isolated bacteria are added (isolated strain-added feed) for 1 month and then fed normal feed for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved growth rate compared to rainbow trout fry usually fed only with normal feed.

### Example 40: Production A5 of Rainbow Trout Fry with Modified Microflora in Gastrointestinal Tract

In the same manner as that of Example 5, bacteria are isolated from an intestinal bacterial flora of an individual exhibiting improved features of essential nutrient (essential fatty acids, essential amino acids, vitamins, and the like) contents. The rainbow trout fry are fed normal feed to which the isolated bacteria are added (isolated strain-added feed) for 1 month and then fed normal feed for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have an improved nutrient (essential fatty acids, essential amino acids, vitamins, and the like) content compared to rainbow trout fry usually fed only with normal feed.

### Example 41: Production A6 of Rainbow Trout Fry with Modified Microflora in Gastrointestinal Tract

In the same manner as that of Example 5, bacteria are isolated from an intestinal bacterial flora of an individual exhibiting improved features of enhancement of immune response ability. The rainbow trout fry are fed normal feed to which the isolated bacteria are added (isolated strain-added feed) for 1 month and then fed normal feed for 5 months to produce rainbow trout fry in which the bacterial flora in the gastrointestinal tract is modified. As a control group, rainbow trout fry fed only with normal feed for 6 months are also produced.

It can be seen that rainbow trout fry fed with the isolated bacteria have enhancement of immune response ability compared to rainbow trout fry usually fed only with normal feed.

### Example 41A: Production of Sea Bream Fry with Modified Microflora in Gastrointestinal Tract

In the same manner as that of Example 5, bacteria are isolated from an intestinal bacterial flora of an individual exhibiting improved features of growth promotion. Sea bream fry are fed normal feed to which isolated bacteria are added (isolated strain-added feed) for 1 month and then fed normal feed for 5 months to produce sea bream fry in which the bacterial flora in the gastrointestinal tract is modified. As a control group, sea bream fry fed only with normal feed for 6 months are also produced.

It can be seen that sea bream fry fed with the isolated bacteria have an improved growth rate compared to sea bream fry usually fed only with normal feed.

### Example 41B: Production of Tuna Fry with Modified Microflora in Gastrointestinal Tract

In the same manner as that of Example 5, bacteria are isolated from an intestinal bacterial flora of an individual exhibiting improved features of growth promotion. Tuna fry are fed normal feed to which isolated bacteria are added (isolated strain-added feed) for 1 month and then fed normal feed for 5 months to produce tuna fry in which the bacterial flora in the gastrointestinal tract is modified. As a control group, tuna fry fed only with normal feed for 6 months are also produced.

It can be seen that tuna fry fed with the isolated bacteria have an improved growth rate compared to tuna fry usually fed only with normal feed.

### Example 41C: Production of Fugu Fry with Modified Bacterial Flora in Gastrointestinal Tract

In the same manner as that of Example 5, bacteria are isolated from an intestinal bacterial flora of an individual exhibiting improved features of growth promotion. Fugu fry are fed normal feed to which isolated bacteria are added (isolated strain-added feed) for 1 month and then fed normal feed for 5 months to produce fugu fry in which the bacterial flora in the gastrointestinal tract is modified. As a control group, fugu fry fed only with normal feed for 6 months are also produced.

It can be seen that fugu fry fed with the isolated bacteria have an improved growth rate compared to fugu fry usually fed only with normal feed.

### Example 41D: Production of Yellowtail Fry with Modified Microflora in Gastrointestinal Tract

In the same manner as that of Example 5, bacteria are isolated from an intestinal bacterial flora of an individual exhibiting improved features of growth promotion. Yellowtail fry are fed normal feed to which isolated bacteria are added (isolated strain-added feed) for 1 month and then fed normal feed for 5 months to produce yellowtail fry in which the bacterial flora in the gastrointestinal tract is modified. As a control group, tuna fry fed only with normal feed for 6 months are also produced.

It can be seen that yellowtail fry fed with the isolated bacteria have an improved growth rate compared to yellowtail fry usually fed only with normal feed.

### Example 41E: Production of Carp Fry with Modified Microflora in Gastrointestinal Tract

In the same manner as that of Example 5, bacteria are isolated from an intestinal bacterial flora of an individual exhibiting improved features of growth promotion. The carp fry are fed normal feed to which the isolated bacteria are added (isolated strain-added feed) for 1 month and then fed normal feed for 5 months to produce carp fry in which the bacterial flora in the gastrointestinal tract is modified. As a control group, carp fry fed only with normal feed for 6 months are also produced.

It can be seen that carp fry fed with the isolated bacteria have an improved growth rate compared to carp fry usually fed only with normal feed.

### Example 42: Identification of Strain from Tuna and Yellowtail Intestinal Tracts

### Taxon Analysis of Tuna and Yellowtail-Derived Bacteria OR Series

The tuna and yellowtail intestine contents were suspended in a phosphate buffer solution, seeded on Luria broth (LB) agar medium, and statically cultured at room temperature for several days. The resulting colonies were randomly isolated as isolated strains.

About 1,500 base pairs, which correspond to almost the entire length of 16S rRNA gene, were amplified from the genomic DNA of the isolated strain by PCR using pan-bacterial common 16S rRNA primer 8F (5′-AGAGTTTGATCMTGGCTCAG-3′) (SEQ ID NO: 2) and 1492R (5′-GGMTACCTTGTTACGACTT-3′) (SEQ ID NO: 3). The base sequence of the DNA fragment having the entire length or about 800 bp on the 5′ end side was determined, and species related to the DNA fragment were searched for by homology search (BLAST) against The National Center for Biotechnology Information (NCBI) 16S rRNA sequence database to identify the genus level. As a result, the microorganisms were identified as Bacillus sp., Microbacterium sp., Lactococcus sp., and Micrococcus sp., respectively.

The sequences of 16S rRNAs of the isolated strains are as shown in SEQ ID NOs: 19 to 22.

### Example 43: Metabolism of Tuna and Yellowtail Intestinal Tract Isolated Strains

The cellulolytic activity of each of the strain isolated in Example 42 and Schewanella sp. GI35 strain (strain E) isolated in Example 1 was measured in the same manner as that of Example 6.

The results are shown in Table 6.

TABLE 6

| Feeding | | | Homology of 165 | |
|---|---|---|---|---|
| Exp. | | Assignment | rRNA | cellulase |
| A | KME43 | Bacillus sp. | Bacillus | + |
| | | | licheniformis (99%) | |
| B | SI20 | Microbacterium | Microbacterium | + |
| | | sp. | moritypicum (99%) | |
| C | SI26 | Lactococcus sp. | Lactococcus | +/− |
| | | | garvieae (99%) | |
| D | Squ08 | Micrococcus sp. | Micrococcus | + |
| | | | aloeverae (99%) | |
| E | GI35 | Shewanella sp. | Shewanella | − |
| | | | putrefaciens (99%) | |

### Example 44: Body Weight Change and Safety Test in Sea Bream Fed with Isolated Strain-Containing Feed

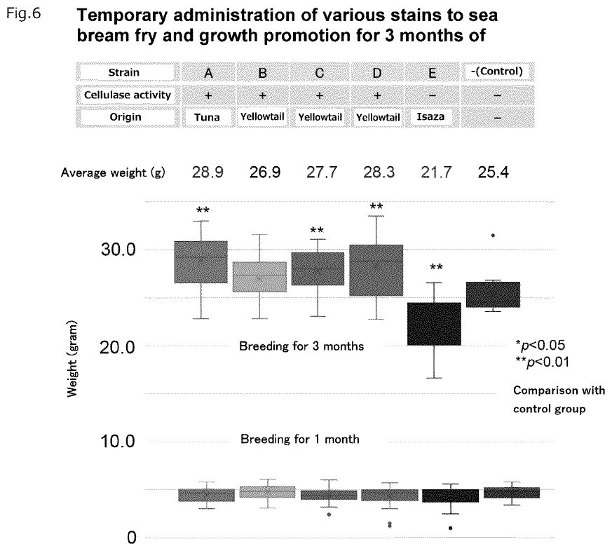

Red sea bream fry about 1 month after hatching were fed normal feed to which each of the isolated strains (isolated strain-added feed) shown in Table 6 was added in an amount corresponding to about 4% of the body weight for 10 days, and fed normal feed for 3 months, and then, the body weights of the red sea bream fry were measured. As a control, breeding was performed by feeding only normal feed for 3 months. The results are shown in FIG. 6. Note that the feed was fed in an amount of 4% of the normal body weight, but the amount of the feed was appropriately adjusted according to the feeding situation.

### (Results)

Red sea bream fed with the tuna-derived isolated strain A and the yellowtail-derived isolated strains B to D gained body weights compared to controls.

### (Note)

Although the present disclosure has been illustrated using preferred embodiments of the present disclosure as described above, it is understood that the scope of the present disclosure should be interpreted only by the claims. It is understood that the patents, patent applications, and other documents cited in the present specification are to be incorporated by reference in the present specification in the same manner as the contents is specifically described in the present specification. The present application claims priority to Japanese Patent Application No. 2021-29165 filed on Feb. 25, 2021 to the Japanese Patent Office, the entire contents of which are incorporated herein by reference in the same manner as if the entire contents constituted the present application.

### INDUSTRIAL APPLICABILITY

Since the method of the present disclosure can provide a novel organism breeding technique, it is possible to develop various organisms in which a bacterial flora is modified, and applications to the agricultural field and the medical field can be expected.

Accession No.

### SEQUENCE LISTING FREE TEXT

SEQ ID NO: 1: Full-length nucleic acid sequence of Pfa operon expected from the whole genome sequence of GI35 strain

SEQ ID NO: 2: Nucleic acid sequence of pan-bacterial common 16S rRNA primer 8F (5′-AGAGTTTGATCMTGGCTCAG-3′)

SEQ ID NO: 3: Nucleic acid sequence of pan-bacterial common 16S rRNA primer 1492R (5′-GGMTACCTTGTTACGACTT-3′)

SEQ ID NO: 4: Nucleic acid sequence of 16S rRNA of Japanese rice fish-derived bacteria OR1

SEQ ID NO: 5: Nucleic acid sequence of 16S rRNA of Japanese rice fish-derived bacteria OR2

SEQ ID NO: 6: Nucleic acid sequence of 16S rRNA of Japanese rice fish-derived bacteria OR3

SEQ ID NO: 7: Nucleic acid sequence of 16S rRNA of Japanese rice fish-derived bacteria OR4

SEQ ID NO: 8: Nucleic acid sequence of 16S rRNA of Japanese rice fish-derived bacteria OR5

SEQ ID NO: 9: Nucleic acid sequence of 16S rRNA of Japanese rice fish-derived bacteria OR6

SEQ ID NO: 10: Nucleic acid sequence of 16S rRNA of Japanese rice fish-derived bacteria OR7

SEQ ID NO: 11: Nucleic acid sequence of 16S rRNA of Japanese rice fish-derived bacteria OR8

SEQ ID NO: 12: Nucleic acid sequence of 16S rRNA of Japanese rice fish-derived bacteria OR12

SEQ ID NO: 13: Nucleic acid sequence of 16S rRNA of Japanese rice fish-derived bacteria OR13

SEQ ID NO: 14: Nucleic acid sequence of 16S rRNA of Japanese rice fish-derived bacteria OR14

SEQ ID NO: 15: Nucleic acid sequence of 16S rRNA of isaza-derived bacteria G12

SEQ ID NO: 16: Nucleic acid sequence of 16S rRNA of isaza-derived bacteria GI431

SEQ ID NO: 17: Nucleic acid sequence of 16S rRNA of isaza-derived bacteria GI71

SEQ ID NO: 18: Nucleic acid sequence of 16S rRNA of isaza-derived bacteria GI83

SEQ ID NO: 19: Nucleic acid sequence of 16S rRNA of tuna-derived isolated strain A (Bacillus sp.)

SEQ ID NO: 20: Nucleic acid sequence of 16S rRNA of yellowtail-derived isolated strain B (Microbacterium sp.)

SEQ ID NO: 21: Nucleic acid sequence of 16S rRNA of yellowtail-derived isolated strain C (Lactococcus sp.)

SEQ ID NO: 22: Nucleic acid sequence of 16S rRNA of yellowtail-derived isolated strain D (Micrococcus sp.)

1.-13. (canceled)

14. A method for producing an improved target organism, the method comprising:
a step A) of selecting an individual exhibiting the improvement from candidate microorganisms of an organism species to which a derived individual different from the target individual belongs;
a step B) of obtaining, in a derived individual exhibiting the improvement, an exogenous microorganism involved in the improvement or a part thereof from a gastrointestinal microflora of the derived individual;
a step C) of introducing the exogenous microorganism or a part thereof into the target individual; and
a step D) of optionally confirming the properties of the gastrointestinal microflora in the target individual, and confirming that a desired improvement is achieved.

15. The method according to claim 14, wherein the step C) includes a step of introducing the exogenous microorganism or a part thereof into the target individual at least at a part of a time during breeding of the target individual.

16. The method according to claim 14, wherein the step C) includes a step of introducing the exogenous microorganism or a part thereof into the target individual at least at a part of a time during breeding of the target individual, and breeding the target individual without administering the microorganism during the rest of the time.

17. The method according to claim 14, wherein the step C) includes a step of introducing the exogenous microorganism or a part thereof into the target individual at least at a part of a time during growing of the target individual.

18. The method according to claim 14, wherein the step C) includes a step of introducing the exogenous microorganism or a part thereof into the target individual at least at a part of a time during growing of the target individual, and breeding the target individual without administering the microorganism during the rest of the time.

19.-24. (canceled)

25. A method for producing an organism having improved or altered nutrient availability, the method comprising
a step of introducing, into a gastrointestinal microflora, a microorganism or enzyme that imparts a metabolic activity of using a component that is not a nutrient source for the organism as a nutrient source for the organism and/or improves the metabolic activity of the organism for the component that is a nutrient source for the organism,
wherein a substance that is not nutritionally available in the organism is allowed to be nutritionally available.

26. The method according to claim 25, wherein the step of introducing the microorganism or enzyme that improves the metabolic activity of the organism into the gastrointestinal microflora includes a step of introducing the microorganism or enzyme that improves the metabolic activity of the organism into the organism at least at a part of a time during breeding of the organism.

27. The method according to claim 25, wherein the step of introducing the microorganism or enzyme that improves the metabolic activity of the organism into the gastrointestinal microflora includes a step of introducing the microorganism or enzyme that improves the metabolic activity of the organism into the organism at least at a part of a time during breeding of the organism, and breeding the organism without administering the microorganism during the rest of the time.

28. The method according to claim 25, wherein the step of introducing the microorganism or enzyme that improves the metabolic activity of the organism into the gastrointestinal microflora includes a step of introducing the microorganism or enzyme that improves the metabolic activity of the organism into the organism at least at a part of a time during growing of the organism.

29. The method according to claim 25, wherein the step of introducing the microorganism or enzyme that improves the metabolic activity of the organism into the gastrointestinal microflora includes a step of introducing the microorganism or enzyme that improves the metabolic activity of the organism into the organism at least at a part of a time during growing of the organism, and breeding the organism without administering the microorganism during the rest of the time.

30. The method according to claim 25, wherein the nutrition includes one or more selected from the group consisting of a fatty acid, a carbon source (carbohydrate), cellulose, hemicellulose, and lignin, an amino acid, a vitamin, a carotenoid, and a mineral.

31. (canceled)

32. A method for producing a target individual of an animal modified to have a component of a photosynthetic organism that is not a nutrient source in the animal as a nutrient source in the animal, the method comprising:
a step A) of providing an exogenous microorganism having an ability to convert the component of the photosynthetic organism into a nutrient source in the animal, or a part thereof; and
a step B) of introducing the exogenous microorganism or a part thereof into the target individual.

33. The method according to claim 32, wherein the step B) includes a step of introducing the exogenous microorganism or a part thereof into the target individual at least at a part of a time during breeding of the target individual.

34. The method according to claim 32, wherein the step B) includes a step of introducing the exogenous microorganism or a part thereof into the target individual at least at a part of a time during breeding of the target individual, and breeding the target individual without administering the microorganism during the rest of the time.

35. The method according to claim 32, wherein the step B) includes a step of introducing the exogenous microorganism or a part thereof into the target individual at least at a part of a time during growing of the target individual.

36. The method according to claim 32, wherein the step B) includes a step of introducing the exogenous microorganism or a part thereof into the target individual at least at a part of a time during growing of the target individual, and breeding the target individual without administering the microorganism during the rest of the time.

37.-74. (canceled)
